(19) United States
(12) Patent Application Publication
(10) Pub. No.: US 2013/0065883 A1
(43) Pub. Date: Mar. 14, 2013

(54) Triazolo [4, 5- B] Pyridin Derivatives

(71) Applicants: Joaquin Pastor Fernández, Madrid (ES); Julen Oyarzabal Santamarina, Madrid (ES); Carl-Gustaf Pierre Saluste, Madrid (ES); Carmen Blanco Aparicio, Madrid (ES); Rosa Maria Alvarez Escobar, Madrid (ES); Virginia Rivero Buceta, Madrid (ES)

(72) Inventors: Joaquin Pastor Fernández, Madrid (ES); Julen Oyarzabal Santamarina, Madrid (ES); Carl-Gustaf Pierre Saluste, Madrid (ES); Carmen Blanco Aparicio, Madrid (ES); Rosa Maria Alvarez Escobar, Madrid (ES); Virginia Rivero Buceta, Madrid (ES)

(73) Assignee: Centro Nacional de Investigaceiones Oncologicas (CNIO), Madrid (ES)

(21) Appl. No.: 13/579,781

(22) PCT Filed: Feb. 18, 2011
(86) PCT No.: PCT/GB2011/000233

§ 371 Date: Nov. 29, 2012

(30) Foreign Application Priority Data
Feb. 18, 2010 (EP) 10380020.7

(51) Int. Cl. C07D 471/04; A61K 31/437; A61K 31/444; A61K 31/4545; A61K 31/5377; A61K 31/537; C07D 471/10; A61K 31/438; C07D 498/10; A61K 31/5386; A61P 35/00; A61P 9/00; A61P 31/12; A61P 29/00; A61P 3/00; A61P 5/00; A61P 25/00; A61P 11/00; A61P 27/14; A61P 37/02; A61K 31/55

(52) U.S. Cl. 514/210.21; 540/597; 514/217.07; 546/117; 514/300; 514/234.2; 544/127; 544/362; 514/253.04; 514/278; 546/16; 544/71; 540/543; 514/212.02

(57) ABSTRACT

There is provided compounds of formula (I), wherein R¹, R², R³ and R⁴ have meanings given in the description, and pharmaceutically-acceptable esters, amides, solvates or salts thereof, which compounds are useful in the treatment of diseases in which inhibition of a protein or lipid kinase (e.g. a PIM family kinase, such as PIM-1, PIM-2 and/or PIM-3, and/or Flt3) is desired and/or required, and particularly in the treatment of cancer or a proliferative disease.

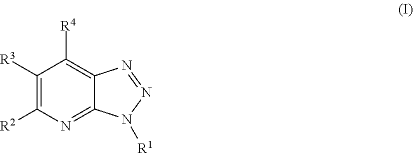

### FIELD OF THE INVENTION

This invention relates to novel pharmaceutically-useful compounds, which compounds are useful as inhibitors of protein or lipid kinases (such as inhibitors of a member of the PIM family kinases, e.g. PIM-1, PIM-2 or PIM-3, or Flt3 inhibitors). The invention also relates to the use of such compounds as medicaments, to the use of such compounds for in vitro, in situ and in vivo diagnosis or treatment of mammalian cells (or associated pathological conditions), to pharmaceutical compositions containing them, and to synthetic routes for their production.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

PIM-1 is the protooncogene activated by murine leucemia virus (Provirus Integration site for Moloney murine leucemia virus—MoMuLV) that induces T-cell lymphoma [Cuypers, H. T., et. al. Cell, 1984, 37, 141-150].

The expression of the protooncogene produces a non-transmembrane serine/threonine kinase of 313 residues, including a kinase domain consisting of 253 amino acid residues. Two isoforms are known through alternative initiation (p44 and p33) [Saris, C. J. M. et al. EMBO J. 1991, 10, 655-664].

PIM-1, PIM-2 and PIM-3 phosphorylate protein substrates that are important in cancer neogenesis and progression. For example, PIM-1 phosphorylates inter alia p21, Bad, c-myb, Cdc 25A and eIF4B (see e.g. Quian, K. C. et al, J. Biol. Chem. 2005, 280(7), 6130-6137, and references cited therein).

Two PIM-1 homologs have been described [Baytel, D. Biochem. Biophys. Acta 1998, 1442, 274-285; Feldman, J. et al. J. Biol. Chem. 1998, 273, 16535.16543]. PIM-2 and PIM-3 are respectively 58% and 69% identical to PIM-1 at the amino acid level. PIM-1 is mainly expressed in thymus, testis, and cells of the hematopoietic system [Mikkers, H.; Nawijn, M.; Allen, J.; Brouwers, C.; Verhoeven, E.; Jonkers, J.; Berns, Mol. Cell. Biol. 2004, 24, 6104; Bachmann, M.; Moroy, T. Int. J. Biochem. Cell Biol. 2005, 37, 726-730. 6115]. PIM-1 expression is directly induced by STAT (Signal Transducers and Activators of Transcription) transcription factors, and PIM-1 expression is induced by many cytokine signalling pathways such as interleukins (IL), granulocyte-macrophage colony stimulating factor (GM-CSF), α- and γ-interferon, erythropoietin, and prolactin [Wang, Z et al. J. Vet. Sci. 2001, 2, 167-179].

PIM-1 has been implicated in lymphoma development. Induced expression of PIM-1 and the protooncogene c-myc synergise to increase the incidence of lymphomagenesis [Breuer, M. et al. Nature 1989, 340, 61-63; van Lohuizen M. et al. Cell, 1991, 65, 737-752]. PIM-1 functions in cytokine signalling pathways and has been shown to play a role in T cell development [Schmidt, T. et al. EMBO J. 1998, 17, 5349-5359; Jacobs, H. et al. JEM 1999, 190, 1059-1068]. Signalling through gp130, a subunit common to receptors of the IL-6 cytokine family, activates the transcription factor STAT3 and can lead to the proliferation of hematopioetic cells [Hirano, T. et al. Oncogene 2000, 19, 2548-2556]. A kinase-active PIM-1 appears to be essential for the gp130-mediated STAT3 proliferation signal. In cooperation with the c-myc PIM-1 can promote STAT3-mediated cell cycle progression and antiapoptosis [Shirogane, T. et sl., immunity, 1999, 11, 709-719]. PIM-1 also appears to be necessary for IL-3-stimulated growth in bone marrow-derived mast cells [Domen, J. et al., Blood, 1993, 82, 1445-1452] and survival of FDCP1 cells after IL-3 withdrawal [Lilly, M. et al., Oncogene, 1999, 18, 4022-4031].

Additionally, control of cell proliferation and survival by PIM-1 may be effected by means of its phosphorylation of the well-established cell cycle regulators cdc25 [Mochizuki, T. et al., J. Biol. Chem. 1999, 274, 18659-18666] and/or p21(Cip1/WAF1) [Wang Z. et al. Biochim. Biophys. Acta 2002, 1593, 45-55] or phosphorylation of heterochromatin protein 1, a molecule involved in chromatin structure and transcriptional regulation [Koike, N. et al, FEBS Lett. 2000, 467, 17-21].

Mice deficient for all three PIM genes showed an impaired response to hematopoietic growth factors and demonstrated that PIM proteins are required for efficient proliferation of peripheral T lymphocytes. In particular, it was shown that PIM function is required for efficient cell cycle induction of T cells in response to synergistic T-cell receptor and IL-2 signalling. A large number of interaction partners and substrates of PIM-1 have been identified, suggesting a pivotal role for PIM-1 in cell cycle control, proliferation, as well as in cell survival.

The oncogenic potential of this kinase has been first demonstrated in E μ PIM-1 transgenic mice in which PIM-1 over-expression is targeted to the B-cell lineage which leads to formation of B-cell tumors [van Lohuizen, M. et al.; Cell 1989, 56, 673-682. Subsequently PIM-1 has been reported to be over-expressed in a number of prostate cancers, erythroleukemias, and several other types of human leukemias [Roh, M. et al.;. Cancer Res. 2003, 63, 8079-8084; Valdman, A. et al; Prostate 2004, 60, 367-371;

For example, chromosomal translocation of PIM-1 leads to overexpression of PIM-1 in diffuse large cell lymphoma. [Akasaka, H. et al.; Cancer Res. 2000, 60, 2335-2341]. Furthermore, a number of missense mutations in PIM-1 have been reported in lymphomas of the nervous system and AIDS-induced non-Hodgkins' lymphomas that probably affect PIM-1 kinase activity or stability [Pasqualucci, L. et al, Nature 2001, 412, 341-346; Montesinos-Rongen, M. et al., Blood 2004, 103, 1869-1875; Gaidano, G. et al., Blood 2003, 102, 1833-184]. Thus, the strong linkage between reported overexpression data and the occurrence of PIM-1 mutations in cancer suggests a dominant role of PIM-1 in tumorigenesis.

Several other protein kinases have been described in the literature, in which the activity and/or elevated activity of such protein kinases have been implicated in diseases such as cancer, in a similar manner to PIM-1, PIM-2 and PIM-3.

For instance, Flt3 kinase (FMS-like tyrosine kinase 3) is a useful target for certain cancers, including leukemia. Flt3 is prevalent in acute myelogenous leukemia (AML) patients, so inhibitors of Flt3 may be useful to treat such patients. Smith et al reported an alkaloid that is a potent inhibitor of Flt3 and provided clinical responses in tested subjects with minimal dose-related toxicity (Blood, vol 103(10), 3669-76 (2004)).

Flt3 inhibitors may also be useful in the treatment of inflammation, as they have been shown to be effective in treating airway inflammation in mice, using a murine asthma model (Edwan et al., J. Immunology, 5016-23 (2004)).

There is a constant need to provide alternative and/or more efficacious inhibitors of protein or lipid kinases, and particularly inhibitors of PIM-1, PIM-2 and/or PIM-3, and/or inhibitors of Flt3. Such modulators are expected to offer alternative and/or improved approaches for the management of medical conditions associated with activity and/or elevated activity of PIM-1, PIM-2 and/or PIM-3 protein kinases.

The listing or discussion of an apparently prior-published document in this specification should not necessarily be taken as an acknowledgement that the document is part of the state of the art or is common general knowledge.

For the treatment of cancer, targeted therapies are becoming more important. That is, therapy that has the effect of interfering with specific target molecules that are linked to tumor growth and/or carcinogenesis. Such therapy may be more effective than current treatments (e.g. chemotherapy) and less harmful to normal cells (e.g. because chemotherapy has the potential to kill normal cells as well as cancerous cells). This, and also the fact that targeted therapies may be selective (i.e. it may inhibit a certain targeted molecule more selectively as compared to other molecular targets, e.g. as described hereinafter), may have the benefit of reducing side effects and may also have the benefit that certain specific cancers can be treated (also selectively). The latter may in turn also reduce side effects.

Hence, it is a clear goal of current oncologists to develop targeted therapies (e.g. ones that are selective). In this respect, it should be pointed out that several different molecular targets may exist that are linked to certain diseases (e.g. cancer). However, one simply cannot predict if a therapy (e.g. a small molecule as a therapeutic) that interferes with or inhibits one target molecule could inhibit a different molecular target (be it one that will ultimately have the effect of treating the same disease or a different one).

European patent application EP 1 082 960 and international patent application WO 98/08847 both disclose inter alia triazolopyridines, which may be useful as medicaments (e.g. for treating depression or other diseases linked to antagonizing CRF). However, there is no disclosure of triazolopyridines that are substituted with an amine at the 5-position, nor does this document disclose that the compounds may be of use as kinase inhibitors. International patent application WO 2005/007658 also discloses various bicyclic compounds, but this document does not predominantly relate to [1,2,3]triazolo[4,5-b]pyridines, nor of the use of the compounds disclosed therein as kinase inhibitors.

European patent application EP 0 773 023 discloses various bicyclic compounds for use in treating inter alia cardiovascular diseases. However, there is no disclosure in that document of [1,2,3]triazolo[4,5-b]pyridines, nor of the use of the compounds disclosed therein as kinase inhibitors.

International patent application WO 2009/038847 discloses compounds that may act as potent antagonists of the CCR9 receptor (and therefore of use in the treatment of e.g. inflammatory conditions). This document relates to aryl sulfonamide compounds, including those attached to a triazolopyridine. However, the specific triazolopyridines disclosed are unsubstituted on the pyridine ring of the triazolopyridine bicycle. Further, this document does not disclose that the compounds mentioned therein may be useful as kinase inhibitors.

International patent applications WO 2009/060197 and WO 2009/040552 disclose various imidazopyridazine-based and imidazolothiadiazolo-based compounds, for use as certain protein kinase inhibitors. However, these documents do not relate to triazolopyridines.

Journal article Bulletin des Societes Chimiques Beiges vol. 97, no. 1, 1988, pages 85-86 by L'abbé, Gerrit et al discloses a general synthetic method for preparing triazolopyridines as well as certain triazolopyridines themselves. However, this document does not disclosure any practical application of the compounds mentioned therein.

European patent application EP 0 773 023 discloses various compounds, including bicycles, which may be useful as corticotrophin releasing factor antagonists (and therefore of potential use in treating e.g. cardiovascular diseases). However, this case mainly relates to monocyclic compounds or bicyclic compounds that are pyrazolopyridines, imidazopyridines or pyrrolopyrimidines. This document also does not suggest that the compounds disclosed therein may be useful as certain kinase inhibitors.

International patent application WO 98/08847 discloses various bicyclic compounds that may exhibit activity as corticotrophin releasing factor antagonists (CFR antagonists), and may therefore be of potential use in the treatment of e.g. stress related illnesses such as mood disorders/depression. However, this document mainly relates to bicyclic compounds that are pyrrolopyrimidines or pyrrolopyridines, in which the 6-membered ring may not be substituted with an amino moiety.

International patent application WO 2006/087538 discloses various 5,6-fused bicyclic compounds that are of potential use as Trk kinase inhibitors and therefore of use in the treatment of certain cancers. However, although the 5,6-fused bicycles may be substituted on the 5-membered ring, such a substituent is necessarily substituted with an alkylene moiety.

French patent application FR 2 915 199 discloses various 5,6-fused bicyclic compounds, including triazolopyridines, which may be useful as inhibitors of the enzyme monoacyl glycerol lipase (MGL) and/or fatty acid amide hydrolase (FAAH) and therefore may be useful in the treatment of e.g. pain. This document does not mention that the compounds may be useful as kinase inhibitors. Further, this document only discloses 5,6-fused bicycles in which the 5-membered ring is substituted with a carbonyl group attached to a non-aromatic heterocycloalkyl group.

International patent application WO 2009/140128 discloses various bicyclic compounds, which may be useful as certain kinase inhibitors. However, this document does not disclose triazolopyridines.

International patent application WO 2007/104053 discloses various bicyclic compounds, which may be useful as Mnk2 inhibitors and therefore of potential use in the treatment of metabolic disorders, such as obesity/diabetes. This document does not disclose triazolopyridines, nor does it disclose such compounds in which the 5-membered ring is substituted on a nitrogen atom with an aromatic group.

International patent application WO 2005/016528 discloses various bicyclic compounds, which may be useful as kinase inhibitors. However, this document does not disclose triazolopyridines.

### DISCLOSURE OF THE INVENTION

According to the invention, there is now provided a compound of formula I,

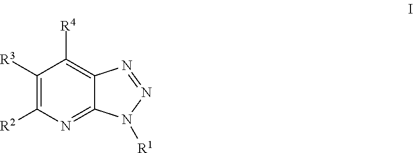

wherein:
R¹ represents aryl or heteroaryl, both of which are optionally substituted by one or more substituents selected from E¹;
R² represents a fragment of formula IA,

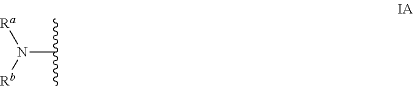

wherein R^{a} and R^{b} independently represent H, —C(O)—C₁₋₁₁ alkyl, —S(O)₂—C₁₋₁₁ alkyl, C₁₋₁₂ (e.g. C₁₋₈) alkyl, heterocycloalkyl (which latter four groups are optionally substituted by one or more substituents selected from ═O, ═NOR^{7a} and Q¹), aryl or heteroaryl (which latter two groups are optionally substituted by one or more substituents selected from Q²); or
R^{a} and R^{b} are linked together, along with the requisite nitrogen atom to which they are necessarily attached, to form a (first) 3- to 7-membered cyclic group, optionally containing one further heteroatom selected from nitrogen, sulfur and oxygen, and which ring optionally:
(a) is fused to a second ring that is either a 3- to 7-membered saturated heterocycloalkyl group containing one to four heteroatoms selected from oxygen, sulfur and nitrogen (preferably oxygen and nitrogen), a 3- to 12-membered saturated carbocyclic ring, or an unsaturated 5- to 12-membered carbocyclic or heterocyclic ring (in which the heteroatoms are preferably selected from sulfur and, especially, nitrogen and oxygen);
(b) comprises a linker group —(C(R^{x})₂)ₚ— and/or —(C(R^{x})₂)ᵣ—O—(C(R^{x})₂)ₛ— (wherein p is 1 or 2; r is 0 or 1; s is 0 or 1; and each R^{x} independently represents hydrogen or C₁₋₆ alkyl), linking together any two non-adjacent atoms of the first 3- to 7-membered ring (i.e. forming a bridged structure); or
(c) comprises a second ring that is either a 3- to 12-membered saturated carbocyclic ring or a 3- to 7-membered saturated heterocycloalkyl group containing one to four heteroatoms selected from oxygen and nitrogen, and which second ring is linked together with the first ring via a single carbon atom common to both rings (i.e. forming a spiro-cycle),
   all of which cyclic groups, defined by the linkage of R^{a} and R^{b}, are optionally substituted by one or more substituents selected from ═O, ═NOR^{7b} and E²;
   R³ and R⁴ independently represent hydrogen or a substituent selected from halo, —CN, R^{j1}, —OR^{j2}, —SR^{j3}, —N(R^{j4})R^{j5} and —C(O)OR^{j6};
   R^{j1}, R^{j2}, R^{j3}, R^{j4}, R^{j5} and R^{j6} independently represent hydrogen or C₁₋₆ (e.g. C₁₋₄) alkyl optionally substituted by one or more substituents selected from halo and —OR^{h};
   R^{h} represents hydrogen or C₁₋₄ alkyl optionally substituted by one or more halo atoms;
   R^{7a} and R^{7b} independently represent hydrogen or C₁₋₆ alkyl optionally substituted by one or more fluoro atoms;
   each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)N(R^{10a})—OR^{11c}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
   each R^{11c} independently represents, on each occasion when used herein, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶);
   each R^{10a}, R^{11a} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶); or
   any relevant pair of R^{10a}, R^{11a} and R^{12a} (for example, when attached to the same atom, adjacent atom (i.e. 1,2-relationship) or to atoms that are two atom atoms apart, i.e. in a 1,3-relationship) may be linked together to form (e.g. along with the requisite nitrogen atom to which they may be attached) a 4- to 20- (e.g. 4- to 12-) membered ring, optionally containing one or more heteroatoms (for example, in addition to those that may already be present, e.g. (a) heteroatom(s) selected from oxygen, nitrogen and sulfur), optionally containing one or more unsaturations (preferably, double bonds), and which ring is optionally substituted by one or more substituents selected from ═O, ═S, ═N(R²⁰) and E⁷;
   each E¹, E², E³, E⁴, E⁵, E⁶ and E⁷ independently represents, on each occasion when used herein:

### (i) Q⁴;

(ii) C₁₋₁₂ alkyl optionally substituted by one or more substituents selected from ═O and Q⁵; or
any two E¹, E², E³, E⁴, E⁵, E⁶ or E⁷ groups, for example on C₁₋₁₂ alkyl groups, e.g. when they are attached to the same or adjacent carbon atoms, or on aryl groups when attached to adjacent carbon atoms, may be linked together to form a 3- to 12-membered ring, optionally containing one or more (e.g. one to three) unsaturations (preferably, double bonds), and which ring is optionally substituted by one or more substituents selected from ═O and J¹;

each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —C(═Y)N(R²⁰)—O—R^{21a}, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);

each Y independently represents, on each occasion when used herein, ═O, ═S, ═NR²³ or ═N—CN;
each R^{21a} independently represents, on each occasion when used herein, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may (for example, when attached to the same atom, adjacent atom (i.e. 1,2-relationship) or to atoms that are two atom atoms apart, i.e. in a 1,3-relationship) be linked together to form (e.g. along with the requisite nitrogen atom to which they may be attached) a 4- to 20- (e.g. 4- to 12-) membered ring, optionally containing one or more heteroatoms (for example, in addition to those that may already be present, e.g. (a) heteroatom(s) selected from oxygen, nitrogen and sulfur), optionally containing one or more unsaturations (preferably, double bonds), and which ring is optionally substituted by one or more substituents selected from J⁶ and ═O;
each J¹, J², J³, J⁴, J⁵ and J⁶ independently represents, on each occasion when used herein:

### (i) Q⁷;

(ii) C₁₋₆ alkyl or heterocycloalkyl, both of which are optionally substituted by one or more substituents selected from ═O and Q⁸;
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰)R⁵¹, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may (for example when attached to the same or adjacent atoms) be linked together to form, a 3- to 8-membered ring, optionally containing one or more heteroatoms (for example, in addition to those that may already be present, heteroatoms selected from oxygen, nitrogen and sulfur), optionally containing one or more unsaturations (preferably, double bonds), and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically acceptable ester, amide, solvate or salt thereof,
provided that when R² represents —NH₂, R³ represents —CN and R⁴ represents hydrogen, then R¹ does not represent unsubstituted phenyl,
which compounds, esters, amides, solvates and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

By “pharmaceutically acceptable ester, amide, solvate or salt thereof”, we include salts of pharmaceutically acceptable esters or amides, and solvates of pharmaceutically acceptable esters, amides or salts. For instance, pharmaceutically acceptable esters and amides such as those defined herein may be mentioned, as well as pharmaceutically acceptable solvates or salts.

Pharmaceutically acceptable esters and amides of the compounds of the invention are also included within the scope of the invention. Pharmaceutically acceptable esters and amides of compounds of the invention may be formed from corresponding compounds that have an appropriate group, for example an acid group, converted to the appropriate ester or amide. For example, pharmaceutically acceptable esters (of carboxylic acids of compounds of the invention) that may be mentioned include optionally substituted C₁₋₆ alkyl, C₅₋₁₀ aryl and/or C₅₋₁₀ aryl-C₁₋₆ alkyl-esters. Pharmaceutically acceptable amides (of carboxylic acids of compounds of the invention) that may be mentioned include those of the formula —C(O)N(R^{z1})R^{z2}, in which R^{z1} and R^{z2} independently represent optionally substituted C₁₋₆ alkyl, C₅₋₁₀ aryl, or C₅₋₁₀ aryl-C₁₋₆ alkylene-. Preferably, C₁₋₆ alkyl groups that may be mentioned in the context of such pharmaceutically acceptable esters and amides are not cyclic, e.g. linear and/or branched.

Further compounds of the invention that may be mentioned include carbamate, carboxamido or ureido derivatives, e.g. such derivatives of existing amino functional groups.

For the purposes of this invention, therefore, prodrugs of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁶I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed herein (e.g. in the description—see routes 1(a), 1 (b), 2(a), 2(b), 2(c) and 2(d)) and/or in the Examples herein below, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

Unless otherwise stated, the term C_{1-q} alkylene (where q is the upper limit of the range) defined herein may be straight-chain or, when there is a sufficient number of carbon atoms, be saturated or unsaturated (so forming, for example, an alkenylene or alkynylene linker group). Such C_{1-q} alkylene groups may be branched (if sufficient number of atoms), but are preferably straight-chained.

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Heterocycloalkyl mentioned herein may be stated to be specifically monocyclic or bicyclic.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of cycloalkyl or heterocycloalkyl groups) refers to monocyclic or bicyclic groups in which two non-adjacent atoms are linked by either an alkylene or heteroalkylene chain (as appropriate).

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydro-naphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group.

It may be specifically stated that the heteroaryl group is monocyclic or bicyclic. In the case where it is specified that the heteroaryl is bicyclic, then it may be consist of a five-, six- or seven-membered monocyclic ring (e.g. a monocyclic heteroaryl ring) fused with another a five-, six- or seven-membered ring (e.g. a monocyclic aryl or heteroaryl ring).

Heteroatoms that may be mentioned include phosphorus, silicon, boron and, preferably, oxygen, nitrogen and sulfur.

For the avoidance of doubt, where it is stated herein that a group (e.g. a C₁₋₁₂ alkyl group) may be substituted by one or more substituents (e.g. selected from E³), then those substituents (e.g. defined by E³) are independent of one another. That is, such groups may be substituted with the same substituent (e.g. defined by E³) or different substituents (defined by E³).

For the avoidance of doubt, in cases in which the identity of two or more substituents in a compound of the invention may be the same, the actual identities of the respective substituents are not in any way interdependent. For example, in the situation in which there is more than one e.g. Q¹ or Q², or, E¹ to E⁷ (such as E⁴) substituent present, then those Q¹ or Q², or, E¹ to E⁷ (e.g. E⁴) substituents may be the same or different. Further, in the case where there are e.g. Q¹ or Q², or, E¹ to E⁷ (such as E⁴) substituents present, in which one represents —OR^{10a} (or e.g. —OR²⁰, as appropriate) and the other represents —C(O)₂R^{10a} (or e.g. —C(O)₂R²⁰, as appropriate), then those R^{10a} or R²⁰ groups are not to be regarded as being interdependent. Also, when e.g. there are two —OR^{10a} substituents present, then those —OR^{10a} groups may be the same or different (i.e. each R^{10a} group may be the same or different).

For the avoidance of doubt, when a term such as “E¹ to E⁷” is employed herein, this will be understood by the skilled person to mean E¹, E², E³, E⁴, E⁵, E⁶ and E⁷, inclusively.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

It is stated hereinbefore that when R^{a} and R^{b} are linked together, then the (first) 3- to 7-membered ring so formed optionally comprises a linker group —(C(R^{x})₂)ₚ— and/or —(C(R^{x})₂)ᵣ—O—(C(R^{x})₂)ₛ— linking together any two non-adjacent atoms of the first ring to form a bridged structure. By this, we include that the first ring may comprise one or more linker groups selected from —(C(R^{x})₂)ₚ— and —(C(R^{x})₂)ᵣ—O—(C(R^{x})₂)ₛ—.

Compounds of the invention that may be mentioned include those in which:

when R^{a} or R^{b} represent (or contain) alkyl (e.g. C₁₋₁₂ alkyl) or heterocycloalkyl, then such groups are optionally substituted by one or more substituents selected from ═O and Q¹;
when R^{a} and R^{b} are linked together to form a ring, then the/those rings formed by the linkage of R^{a} and R^{b} (i.e. the first and optional second rings) are optionally substituted by one or more substituents selected from ═O and E²;
each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³); and/or
each Q⁷ and Q⁵ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂R⁵⁰, —SR⁵⁰, —S(O)R⁵⁰ or C₁₋₆ alkyl optionally substituted by one or more fluoro atoms.

Compounds of the invention that may be mentioned include those in which:
(i) both of R^{a} and R^{b} do not represent hydrogen, i.e. at least one of R^{a} and R^{b} represents a substituent other than hydrogen;
(ii) R³ represents hydrogen or a substituent selected from halo, R^{j1}, —OR^{j2}, —SR^{j3}, —N(R^{j4})R^{j5} and —C(O)OR^{j6} (more preferably, R³ represents —SR^{j3} or, particularly, hydrogen, halo, R^{j1}, —OR^{j2} or —N(R^{j4})R^{j5}).

Other compounds of the invention that may be mentioned include those in which:

when R¹ represents phenyl, then it is substituted by at least one substituent as defined herein;
R¹ does not represent unsubstituted phenyl; and/or
R⁴ does not represent hydrogen (i.e. R⁴ represents a substituent other than hydrogen).

Other compounds of the invention that may be mentioned include those in which, for example when R¹ represents a 6-membered aromatic ring such as phenyl or pyridyl (e.g. 2-pyridyl):

R¹ is not substituted at the ortho-position with an E¹ substituent, in which E¹ represents Q⁴ and Q⁴ represents —N(R²²)S(O)₂R²⁰ (e.g. in which R²⁰ represents heteroaryl or, preferably, aryl);
when E¹ represents Q⁴, then Q⁴ preferably does not represent —N(R²²)S(O)₂R²⁰ (e.g. in which R²⁰ represents heteroaryl or, preferably, aryl).

Other compounds of the invention that may be mentioned include those in which:

when one of R^{a} and R^{b} represents H, then the other does not represent aryl or heteroaryl (in particular, it does not represent heteroaryl, such as a 5-membered heteroaryl ring containing one or two heteroatoms, e.g. a pyrazolyl group such as 3-pyrazolyl);
R^{a} and R^{b} do not represent heteroaryl (in particular, a 5-membered heteroaryl group containing one or two heteroatoms, e.g. a pyrazolyl group such as 3-pyrazolyl).

Other compounds of the invention that may be mentioned include those in which when R^{a} and R^{b} are linked together as hereinbefore defined, the cyclic group(s) so formed may be substituted by one or more substituents selected from ═O and E², but preferably:

the (first) cyclic group formed by the linkage of R^{a} and R^{b} is not substituted at the ortho- or 2-position (i.e. a to the point of attachment of the cyclic amino group to the requisite bicyclic group of formula I), for instance by an E² group, in which E² represents Q⁴ and Q⁴ represents optionally substituted aryl or heteroaryl; when E² represents Q⁴, then Q⁴ does not represent aryl or heteroaryl.

Preferred compounds of the invention that may be mentioned include those in which:

R^{a} and R^{b} independently represent H, C₁₋₁₂ (e.g. C₁₋₈) alkyl, heterocycloalkyl (which latter four groups are optionally substituted by one or more substituents selected from ═O and Q¹), aryl or heteroaryl (which latter two groups are optionally substituted by one or more substituents selected from Q²); or R^{a} and R^{b} are linked together as hereinbefore defined.

Preferred compounds of the invention that may be mentioned include those in which:

(A) for instance, when R¹ represents a 5-membered or, preferably, a 6-membered aryl or heteroaryl group (e.g. pyridyl, such as 2-pyridyl, or, preferably, phenyl), then, preferably:
(i) that 6-membered aromatic group (e.g. phenyl) may not be substituted (e.g. at the ortho position; relative to the point of attachment to the requisite triazolopyridine bicycle) with E¹, in which E¹ represents —NR²²S(O)₂R²⁰ (and R²⁰ preferably represents aryl or heteroaryl; optionally substituted as defined herein);
(ii) when E¹ represents Q⁴, then Q⁴ may not represent —NR²²S(O)₂R²⁰ (as defined above), for instance, when E¹ represents Q⁴, then Q⁴ is selected from halo, —CN, —NO₂, —N(R²⁰)R²¹, —OR²⁰, —C(═Y)—R²⁰, —C(═Y)—OR²⁰, —C(═Y)N(R²⁰)R²¹, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³); and/or
(iii) when that 6-membered aromatic group (e.g. phenyl) is substituted (e.g. at the ortho position; relative to the point of attachment to the requisite triazolopyridine bicycle) with E¹, then when E¹ represents Q⁴, then Q⁴ may not represent —NR²²S(O)₂R²⁰ (as defined above), for instance, when E¹ represents Q⁴, then Q⁴ is selected from halo, —CN, —NO₂, —N(R²⁰)R²¹, —OR²⁰, —C(═Y)—R²⁰, —C(═Y)—OR²⁰, —C(═Y)N(R²⁰)R²¹, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
(B) for instance, when E¹ represents Q⁴, then, preferably:
(i) Q⁴ may not represent —NR²²S(O)₂R²⁰ (as defined above, e.g. in which R²⁰ represents optionally substituted aryl or heteroaryl), for instance, when E¹ represents Q⁴, then Q⁴ is selected from halo, —CN, —NO₂, —N(R²⁰)R²¹, —OR²⁰, —C(═Y)—R²⁰, —C(═Y)—OR²⁰, —C(═Y)N(R²⁰)R²¹, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
(ii) R¹ may not represent a 5-membered or, especially, a 6-membered aromatic group (e.g. pyridyl, such as 2-pyridyl, or preferably, phenyl) substituted (e.g. at the ortho position; relative to the point of attachment to the requisite triazolopyridine bicycle) with E¹, in which E¹ represents Q⁴ as defined above;
(iii) R²⁰ preferably represents hydrogen, C₁₋₆ alkyl or heterocycloalkyl (which latter two groups are optionally substituted as defined herein).

Further compounds of the invention that may be mentioned include those in which:

when R³ or R⁴ (e.g. R⁴) represents —OR^{j2}, —SR^{j3} or —N(R^{j4})R^{j5}, then those R^{j2}, R^{j3}, R^{j4} and R^{j5} groups preferably do not contain a cyclic moiety (i.e. they represent hydrogen or acyclic C₁₋₆ alkyl optionally substituted by one or more substituents selected from halo and —OR^{h});
R^{j1}, R^{j2}, R^{j3}, R^{j4}, R^{j5} and R^{j6} (e.g. R^{j2}, R^{j3}, R^{j4} and R^{j5}) independently represent hydrogen or acyclic C₁₋₆ alkyl optionally substituted by one or more substituents selected from halo and —OR^{h};
R⁴ preferably represents halo, —CN, R^{j1}, —C(O)OR^{j6} or, more preferably, hydrogen.

Preferred aryl groups and heteroaryl groups (when such heteroaryl groups are bicyclic, they are preferably attached to the requisite triazolopyridazine of formula I via a fused aromatic (e.g. benzene) ring) that R¹ may represent include optionally substituted phenyl, naphthyl, pyrrole, pyrazole, triazole, tetrazole, thiazole, isothiazole, oxazole, isoxazole, isoindole, 1,3-dihydro-indol-2-one, pyridine-2-one, pyridine, pyridine-3-ol, imidazole, 1H-indazole, 1H-indole, indolin-2-one, 1-(indolin-1-yl)ethanone, pyrimidine, pyridazine, pyrazine, isatin groups, 1H-benzo[d][1,2,3]triazole, 1H-pyrazolo[3,4-b]pyridine, 1H-pyrazolo[3,4-d]pyrimidine, 1H-benzo[d]imidazole, 1H-benzo[d]imidazol-2(3H)-one, 1H-pyrazolo[3,4-c]pyridine, 1H-pyrazolo[4,3-d]pyrimidine, 5H-pyrrolo[3,2-d]pyrimidine, 2-amino-1H-purin-6(9H)-one, quinoline, quinazoline, quinoxaline, isoquinoline, isoquinolin-1(2H)-one, 3,4-dihydroisoquinolin-1(2H)-one, 3,4-dihydroquinolin-2(1H)-one, quinazolin-2(1H)-one, quinoxalin-2(1H)-one, 1,8-napthyridine, pyrido[3,4-d]pyrimidine, pyrido[3,2-b]pyrazine, 1,3-dihydro benzimidazolone, benzimidazole, benzothiazole and benzothiadiazole, groups.

Preferred monocyclic heteroaryl groups that R¹, R^{a} or R^{b} or Q¹, Q², Q⁴ or Q⁵ (if applicable) may independently represent include 5- or 6-membered rings, containing one to three (e.g. one or two) heteroatoms selected from sulfur, oxygen and nitrogen. Preferred bicyclic heteroaryl groups that R¹ (e.g. when attached to be requisite bicycle of formula I via a benzene ring of the bicycle), R^{a} or R^{b}, or Q¹, Q², Q⁴ or Q⁵ may represent include 8- to 12- (e.g. 9- or 10-) membered rings containing one to four (e.g. one to three, or, preferably, one or two) heteroatoms selected from sulfur, oxygen and nitrogen (e.g. an indolyl group). Further, bicyclic rings may consist of benzene rings (and bicyclic heteroaryl groups that R¹ may preferably comprise a benzene ring) fused with a monocyclic heteroaryl group (as hereinbefore defined), e.g. a 6- or, preferably 5-membered monocyclic heteroaryl group optionally containing two, or, preferably, one heteroatom selected from sulfur, oxygen and nitrogen.

Preferred heterocycloalkyl groups that R^{a} or R^{b} or Q¹, Q², Q⁴ or Q⁵ may independently represent include 4- to 8-membered (e.g. 4-, 5-, 6- or 7-membered) heterocycloalkyl groups, which groups preferably contain one or two heteroatoms (e.g. sulfur or, preferably, nitrogen and/or oxygen heteroatoms), so forming for example, an optionally substituted azetidinyl, pyrrolidinyl, piperidinyl, morpholinyl or tetrahydropyranyl group.

Preferred C₃₋₆ cycloalkyl groups that R^{a} or R^{b} or Q¹, Q², Q⁴ or Q⁵ may independently represent include optionally substituted C₃₋₈ (e.g. C₃₋₆) cycloalkyl groups, such as cyclohexyl, cyclopentyl, cyclobutyl and cyclopropyl.

Preferred compounds of the invention include those in which when R¹ represents aryl (e.g. phenyl) or heteroaryl (e.g. a 5- or 6-membered heteroaryl group) (but especially when R¹ represents aryl, such as phenyl), then that group may be unsubstituted but is preferably substituted by at least one (e.g. two or, preferably, one) substituent(s) selected from E¹, or, the aryl/heteroaryl (e.g. phenyl) group may be substituted with two E¹ substituents that are linked together, so forming e.g. a bicyclic heteroaryl (e.g. a 8-, 9- or 10-membered heteroaryl group), consisting of a 5- or 6-membered heteroaryl group or, preferably, a 6-membered benzene ring (which is attached to the requisite bicycle of formula I) fused to another 5- or 6-membered ring (in which the latter ring may contain one or more (e.g. four, or, preferably one to three) heteroatoms), and which bicyclic ring system is optionally substituted by one or more (e.g. two or, preferably, one) substituent(s) selected from E¹ or J¹ (as appropriate) (and, if there is a non-aromatic ring present in the bicyclic heteroaryl group, then such a group may also be substituted by one or more (e.g. one) ═O groups).

Further preferred compounds of the invention include those in which:

each Q¹ and Q² independently represent halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —S(O)₂R^{10a}, —SR^{10a}, —S(O)R^{10a}, C₁₋₆ alkyl (optionally substituted by one or more fluoro atoms), aryl or heteroaryl (optionally substituted by one to three (e.g. one) substituent(s) selected from E³, but preferably unsubstituted);
each R^{10a}, R^{11a} and R^{12a} independently represent, on each occasion when used herein, hydrogen or C₁₋₁₂ (e.g. C₁₋₆) alkyl (which latter group is optionally substituted by one or more substituents selected from ═O and E⁵); or
any relevant pair of R^{10a}, R^{11a} and R^{12a} may be linked together as defined herein (although they are preferably not linked);
R^{11c} represents C₁₋₁₂ (e.g. C₁₋₆) alkyl (which latter group is optionally substituted by one or more substituents selected from ═O and E⁵);
each of E¹, E², E³, E⁴, E⁵, E⁶ and E⁷ independently represent, on each occasion when used herein, Q⁴ or C₁₋₆ alkyl (e.g. C₁₋₃) alkyl optionally substituted by one or more substituents selected from ═O and Q⁵;
each Q⁴ and Q⁵ independently represent halo, —CN, —NO₂, —N(R²⁰)R²¹, —OR²⁰, —C(═Y)—R²⁰, —C(═Y)—OR²⁰, —C(═Y)N(R²⁰)R²¹, —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —S(O)₂R²⁰, —SR²⁰, —S(O)R²⁰, C₁₋₆ alkyl (optionally substituted by one or more fluoro atoms), aryl or heteroaryl (optionally substituted by one to three (e.g. one) substituent(s) selected from J³, but preferably unsubstituted);
two E¹ substituents may be linked together as defined herein, but any two E¹, E², E³, E⁴, E⁵, E⁶ and E⁷ are preferably not linked together;
each R²⁰, R²¹, R²² and R²³ independently represent, on each occasion when used herein, aryl (e.g. phenyl; preferably unsubstituted, but which may be substituted by one to three J⁵ groups) or, more preferably, hydrogen or C₁₋₆ (e.g. C₁₋₃) alkyl optionally substituted by one or more substituents selected from ═O and J⁴; or
any pair of R²⁰ and R²¹, may, when attached to the same nitrogen atom, be linked together to form a 4- to 8-membered (e.g. 5- or 6-membered) ring, optionally containing one further heteroatom selected from nitrogen and oxygen, optionally containing one double bond, and which ring is optionally substituted by one or more substituents selected from J⁶ and ═O;
R^{21a} represents C₁₋₆ (e.g. C₁₋₃) alkyl optionally substituted by one or more substituents selected from ═O and J⁴;
each J¹, J², J³, J⁴, J⁵ and J⁶ independently represents C₁₋₆ alkyl (e.g. acyclic C₁₋₄ alkyl or C₃₋₆ cycloalkyl) optionally substituted by one or more substituents selected from ═O and Q⁸, or, such groups independently represent a substituent selected from Q⁷;
each Q⁷ and Q⁸ independently represents a substituent selected from halo (e.g. fluoro), —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂R⁵⁰ or C₁₋₆ alkyl optionally substituted by one or more fluoro atoms;
each R⁵⁰, R⁵¹, R⁵² and R⁵³ substituent independently represents, on each occasion when used herein, hydrogen or C₁₋₆ (e.g. C₁₋₃) alkyl optionally substituted by one or more substituents selected from fluoro;
when any relevant pair of R⁵⁰, R⁵¹ and R⁵² are linked together, then those pairs that are attached to the same nitrogen atom may be linked together (i.e. any pair of R⁵⁰ and R⁵¹), and the ring so formed is preferably a 5- or 6-membered ring, optionally containing one further nitrogen or oxygen heteroatom, and which ring is optionally substituted by one or more substituents selected from ═O and C₁₋₃ alkyl (e.g. methyl);
R⁶⁰, R⁶¹ and R⁶² independently represent hydrogen or C₁₋₃ (e.g. C₁₋₂) alkyl optionally substituted by one or more fluoro atoms.

Preferred optional substituents on R¹ (and also on certain R^{a} and/or R^{b} groups, e.g. when they represent, or contain, aryl, heteroaryl, heterocycloalkyl and/or cycloalkyl groups, then on those substituents/part-substituents):

═O (if applicable, e.g. unless the group is aromatic);

### —CN;

halo (e.g. fluoro, chloro or bromo);
C₁₋₆ (e.g. C₁₋₄) alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl) or substituted with an aryl, heteroaryl or heterocycloalkyl group (which themselves may be substituted with one or more —OR^{z1}, —C(O)R^{z2}, —C(O)OR^{z3}, —N(R^{z4})R^{z5}, —S(O)₂R^{z6}, —S(O)₂N(R^{z7})R^{z8}; —N(R^{z9})—C(O)—R^{z10}, —N(R^{z9})—S(O)₂—R^{z10}, —C(O)—N(R^{z11})R^{z12}, —N(R^{z9})—S(O)₂—N(R^{z10}) and/or —N(R^{z9})—C(O)—N(R^{z10}) substituents;
aryl (e.g. phenyl) (e.g. which substitutent may also be present on an alkyl group, thereby forming e.g. a benzyl group);

### —OR^{z1};

### —C(O)R^{z2};

### —C(O)OR^{z3};

### —N(R^{z4})R^{z5};

### —S(O)₂R^{z6};

### —S(O)₂N(R^{z7})R^{z8};

### —N(R^{z9})—C(O)—R^{z10};

### —N(R^{z9})—S(O)₂—R^{z10};

### —C(O)—N(R^{z11})R^{z12};

### —N(R^{z9})—C(O)—N(R^{z10});

### —N(R^{z9})—S(O)₂—N(R^{z10});

wherein each R^{z1} to R^{z12} independently represents, on each occasion when used herein, H or C₁₋₄ alkyl (e.g. ethyl, n-propyl, t-butyl or, preferably, n-butyl, methyl, isopropyl or cyclopropylmethyl (i.e. a part cyclic alkyl group)) optionally substituted by one or more halo (e.g. fluoro) groups (so forming e.g. a trifluoromethyl group). Further, any two R^{z} groups (e.g. R^{z4} and R^{z5}), when attached to the same nitrogen heteroatom may also be linked together to form a ring such as one hereinbefore defined in respect of corresponding linkage of R^{10a} and R^{11a} groups.

Preferred compounds of the invention include those in which:

each R^{10a}, R^{11a} and R^{12a} independently represent phenyl (optionally substituted by one or more E⁶ substituents), preferably, heterocycloalkyl (optionally substituted by one or more ═O and/or E⁵ substituents) and, more preferably, hydrogen or C₁₋₁₂ (e.g. C₁₋₆) alkyl (optionally substituted by one or more ═O and/or E⁵ substituents), or any pair of R^{10a}, R^{11a} and R^{12a} (e.g. any pair of R^{10a} and R^{11a} when attached to the same nitrogen atom) may be linked together to form a 4- to 10-membered (e.g. a 4- to 6-membered monocyclic) ring, optionally substituted by one or more substituents selected from ═O and E⁷;
each E¹, E², E³, E⁴, E⁵, E⁶ and E⁷ independently represents C₁₋₁₂ alkyl optionally substituted by one or more substituents selected from ═O and Q⁵, or, each E¹ to E⁷ independently represent Q⁴; or, any two E¹ to E⁷ substituents (e.g. when attached to the same or adjacent atoms) may be linked together to form a 3- to 8-membered ring, optionally containing one to three double bonds, one to three heteroatoms, and which ring may be substituted by one or more substituents selected from ═O and J¹;
each R²⁰, R²¹, R²² and R²³ (e.g. each R²⁰ and R²¹) independently represents heteroaryl, preferably, aryl (e.g. phenyl) (which latter two groups are optionally substituted by one or more substituents selected from J⁵), or, more preferably, hydrogen or C₁₋₆ (e.g. C₁₋₄) alkyl optionally substituted by one or more substituents selected from ═O and J⁴; or
any relevant pair of R²⁰, R²¹ and R²² (e.g. R²⁰ and R²¹) may (e.g. when both are attached to the same nitrogen atom) may be linked together to form a 3- to 8- (e.g. 4- to 8-) membered ring, optionally containing a further heteroatom, and optionally substituted by one or more substituents selected from ═O and J⁶;
each J¹, J², J³, J⁴, J⁵ and J⁶ independently represent C₁₋₆ alkyl (e.g. C₁₋₄ acyclic alkyl or C₃₋₅ cycloalkyl) optionally substituted by one or more substituents selected from Q⁸, or, J¹ to J⁶ more preferably represent a substituent selected from Q⁷;
each R⁵⁰, R⁵¹, R⁵² and R⁵³ independently represents hydrogen or C₁₋₆ (e.g. C₁₋₄) alkyl optionally substituted by one or more fluoro atoms;
each R⁶⁰, R⁶¹ and R⁶² independently represents hydrogen or C₁₋₂ alkyl (e.g. methyl).

More preferred compounds of the invention include those in which:

when R^{a} and R^{b} are linked together, they may represent a 3- to 7-membered ring (e.g. a 4- to 7-membered ring), optionally containing one further heteroatom selected from nitrogen and oxygen, which ring may be: (a) fused to another saturated 4- to 6-membered carbocyclic or heterocyclic ring, in which the latter contains one to four heteroatoms preferably selected from nitrogen and oxygen;
or (b) comprises a further 4- to 6-membered saturated carbocyclic or heterocyclic ring, in which the latter contains one or two heteroatoms preferably selected from nitrogen and oxygen, which second ring is linked to the first via a single atom;
Q⁴ and Q⁵ independently represent —S(O)₂R²⁰ or, preferably, halo (e.g. fluoro), —OR²⁰, —N(R²⁰)R²¹, —C(═Y)R²⁰, —C(═Y)OR²⁰, —C(═Y)N(R²⁰)R²¹, —N(R²²)C(═Y)R²¹, —NR²²S(O)₂R²⁰, —S(O)₂N(R²⁰)R²¹, heterocycloalkyl, aryl, heteroaryl (which latter three groups are optionally substituted with one or more substitutents selected from J² or J³, as appropriate) and/or C₁₋₆ alkyl (e.g. C₁₋₃ alkyl) optionally substituted by one or more fluoro atoms;
each Y and Y^{a} independently represents, on each occasion when used herein, ═S, or preferably ═O;
each R²⁰, R²¹, R²² and R²³ (e.g. each R²⁰ and R²¹) independently represents hydrogen or C₁₋₄ (e.g. C₁₋₃) alkyl (e.g. C₁₋₄ acyclic alkyl group or a part cyclic C₄ group) optionally substituted (but preferably unsubstituted) by one or more (e.g. one) J⁴ substituent(s); or
any relevant pair of R²⁰, R²¹ and R²² (e.g. R²⁰ and R²¹) may (e.g. when both are attached to the same nitrogen atom) may be linked together to form a 5- or, preferably, a 6-membered ring, optionally containing a further heteroatom (preferably selected from nitrogen and oxygen), which ring is preferably saturated, and optionally substituted by one or more substituents selected from ═O and J⁶;
R²² represents C₁₋₃ alkyl or hydrogen;
each J¹, J², J³, J⁴, J⁵ and J⁶ independently represent a substituent selected from Q⁷, or J¹ to J⁶ independently represent C₁₋₆ alkyl (e.g. C₁₋₄ alkyl);
each Q⁷ and Q⁸ independently represent halo (e.g. fluoro), —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹ or C₁₋₆ alkyl optionally substituted by one or more fluoro atoms;
each Y^{a} independently represents ═S or, preferably, ═O;
each R⁵⁰, R⁵¹, R⁵² and R⁵³ independently represents H or C₁₋₄ alkyl (e.g. tBu, Me).

Preferred compounds of the invention include those in which:

R¹ represents a 5- or 6-membered heteroaryl group (optionally substituted as defined herein) or, especially, aryl (e.g. phenyl) optionally substituted by one or more (e.g. one to three) substituent(s) selected from E¹, in which the E¹ substituents are as herein defined (or, two E¹ substituents on the aryl (e.g. phenyl) ring may be linked together as defined herein);
R^{j1}, R^{j2}, R^{j3}, R^{j4}, R^{j5} and R^{j6} independently represent hydrogen or preferably C₁₋₄ (e.g. C₁₋₂) alkyl (e.g. ethyl);
Q¹ and Q² (e.g. Q¹) independently represent(s) —OR^{10a}; —N(R^{10a})R^{11a}; —C(═Y)N(R^{10a})R^{11a}; —N(R^{12a})C(═Y)R^{11a}; —C(═Y)OR^{10a}; —S(O)₂R^{10a}; aryl (e.g. phenyl) or heteroaryl (e.g. a monocyclic 5- or 6-membered heteroaryl group, preferably containing one or two (e.g. one) heteroatom(s) preferably selected from nitrogen, so forming e.g. a 3-pyridyl group), both of which are optionally substituted by one or more (e.g. one) substituents selected from E⁴; heterocycloalkyl (e.g. a 5- or 6-membered monocyclic heterocycloalkyl group, preferably containing one or two heteroatoms preferably selected from nitrogen and oxygen, so forming e.g. 4-piperidinyl, piperazinyl or imidazolidinyl) optionally substituted by one or more (e.g. one) substituent(s) selected from ═O and E³ (so forming e.g. optionally substituted piperazin-2-ones or imidazolidin-2-ones); or C₃₋₆ cycloalkyl (e.g. cyclopropyl or cyclohexyl), which group may be unsubstituted (e.g. in the case of cyclopropyl) or is optionally substituted by one or more E³ substituents;
R^{10a}, R^{11a} and R^{12a} independently represent H or C₁₋₆ (e.g. C₁₋₄) alkyl optionally substituted by one or more groups selected from ═O and E⁵;
E¹ to E⁷ independently represent Q⁴ or C₁₋₆ (e.g. C₁₋₃, such as methyl) alkyl optionally substituted by one or more Q⁵ substituents;
any two E¹ to E⁷ substituents are not linked together;
Q⁴ and Q⁵ independently represent aryl (optionally substituted as defined herein), preferably, —S(O)₂R²⁰, —N(R²²)—S(O)₂R²⁰ or, more preferably, halo, —CN, —OR²⁰, —N(R²⁰)R²¹, —C(═Y)R²⁰, —C(═Y)OR²⁰, —N(R²²)C(═Y)R²¹, —C(═Y)N(R²⁰)R²¹, —S(O)₂N(R²⁰)R²¹ (e.g. —S(O)₂NH₂), C₁₋₆ alkyl (e.g. acyclic alkyl or C₃₋₆ cycloalkyl;
which alkyl group is optionally substituted by one or more ═O and/or J² substituents, but preferably, unsubstituted), heterocycloalkyl (e.g. 5- or 6-membered monocyclic heterocycloalkyl group, preferably containing one or two heteroatoms, preferably selected from nitrogen, so forming e.g. piperidinyl) or heteroaryl (e.g. a 5- or 6-membered monocyclic heteroaryl group, preferably containing one or two heteroatoms, preferably selected from nitrogen, so forming e.g. 4-pyridyl; and which heteroaryl group is preferably unsubstituted);
more preferably, Q⁴ represents —S(O)₂R²⁰, —N(R²²)—S(O)₂R²⁰ or, especially, halo (e.g. fluoro), —OR²⁰, —N(R²²)—C(═Y)—R²¹, —C(═Y)OR²⁰, —S(O)₂N(R²⁰)R²¹ or C₁₋₆ (e.g. C₁₋₂) alkyl (e.g. methyl) optionally (and preferably) substituted by one J² substituent;
more preferably, Q⁵ represents aryl (optionally substituted as defined herein), preferably, halo (e.g. fluoro), —C(═Y)N(R²⁰)R²¹, —N(R²²)C(═Y)R²¹, —C(═Y)—R²⁰, C₁₋₆ alkyl (preferably C₃₋₆ cycloalkyl) or heteroaryl (e.g. a 5- or 6-membered monocyclic heteroaryl group, preferably containing one or two heteroatoms, preferably selected from nitrogen, so forming e.g. 4-pyridyl; and which heteroaryl group is preferably unsubstituted);
Y and Y^{a} independently represent ═S or, preferably, ═O;
R²⁰ and R²¹ independently represent hydrogen, C₁₋₄ alkyl, which latter group is optionally substituted by one or more (e.g. one) substituent(s) selected from J⁴;
when there is a —N(R²⁰)R²¹ moiety present, then one of R²⁰ and R²¹ represents hydrogen, and the other represents hydrogen or C₁₋₄ alkyl (e.g. methyl, ethyl or isopropyl), which latter group is optionally substituted by one or more (e.g. one) substituent(s) selected from J⁴;
R²² represents hydrogen and C₁₋₃ alkyl (e.g. methyl);
J¹ to J⁶ (e.g. J² and J⁴) independently represent Q⁷ (or such groups, e.g. J⁴, may also represent C₁₋₆ (e.g. C₁₋₃) alkyl, which is preferably unsubstituted);
Q⁷ and Q⁸ (e.g. Q⁸) independently represent halo (e.g. fluoro), —C(═Y^{a})N(R⁵⁰)R⁵¹ or —C(═Y^{a})—R⁵⁰;
R⁵⁰ and R⁵¹ independently represent C₁₋₆ (e.g. C₁₋₄) alkyl.

Preferred R¹ groups of the compounds of the invention include unsubstituted phenyl, methoxyphenyl (e.g. 4-methoxyphenyl), trifluoromethoxyphenyl (e.g. 3-OCF₃-phenyl), trifluoromethylphenyl (e.g. 3-trifluoromethylphenyl), halophenyl (e.g. fluorophenyl, such as 4-fluorophenyl), cyanophenyl (e.g. 3-cyanophenyl), indolyl (attached to the requisite bicycle via the benzene ring, e.g. 4- or, preferably, 5-indolyl), hydroxyphenyl (e.g. 4-hydroxyphenyl) and amidophenyl (e.g. 4-[(—N(H)—C(O)—CH₃)phenyl]). The phenyl group attached to the requisite triazolopyrazine bicycle of formula I is preferably substituted. Preferably substituents on such phenyl groups are in the meta and/or para position (or two substituents in the meta and para position may be linked together to form a further ring, e.g. an indolyl ring). When R¹ represents phenyl, other groups that may be mentioned include aminophenyl groups (e.g. R¹ may also represent 3-(N(CH₃)₂-phenyl)). Other R¹ groups that may be mentioned include optionally substituted 5- or 6-membered heteroaryl groups, preferably containing one or two heteroatoms, for instance R¹ may represent: optionally substituted pyridyl (e.g. 3-pyridyl or 4-pyridyl, such 2-trifluoromethyl-4-pyridyl or 6-amino-3-pyridyl), which may be substituted by one or two substituents in which there is preferably one substituent at the position meta or para relative to its point of attachment to the requisite bicycle of formula I; and optionally substituted thiazolyl (e.g. 2-thiazolyl, such as 4-trifluoromethyl-2-thiazolyl).

Particularly preferred R¹ groups include optionally substituted phenyl (in which the optional substituent E¹ is preferably in the para or preferably meta position and preferably represents —OR²⁰, —N(R²⁰)R²¹ or C₁₋₂ alkyl (e.g. methyl) optionally substituted by one or more fluoro atoms, so forming e.g. a —CF₃ group).

Preferred compounds of the invention include those in which R² represents one of the following fragments:

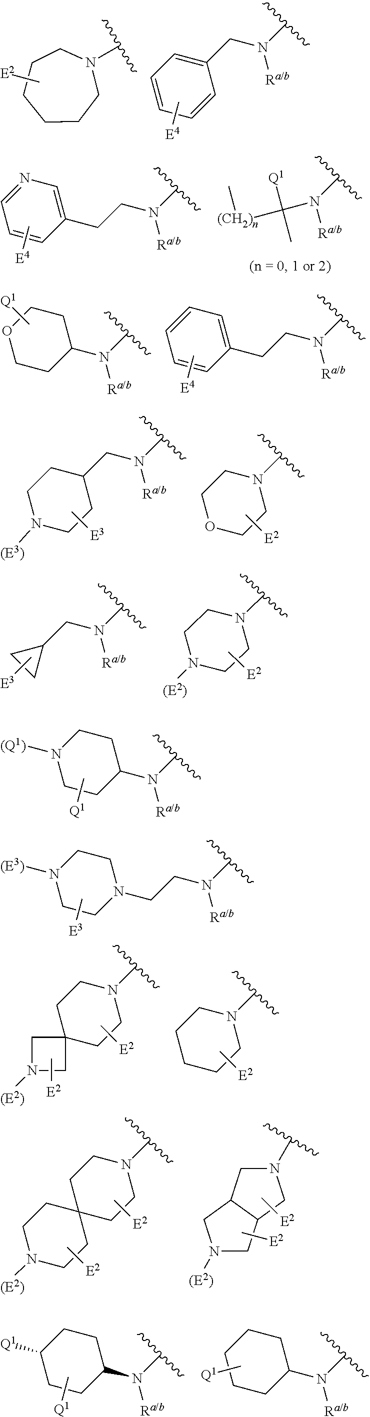

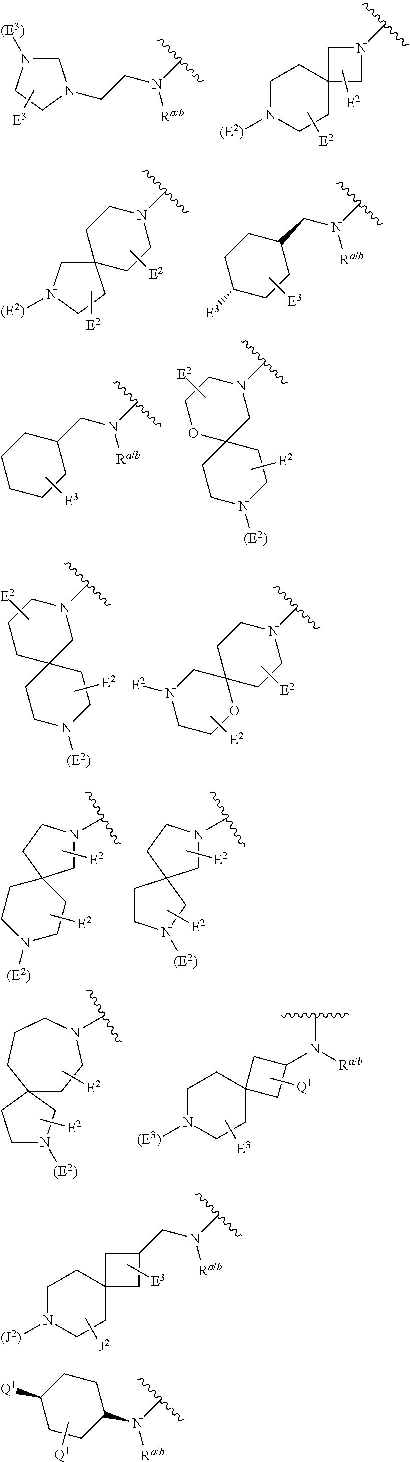

wherein the squiggly line represents the point of attachment to the requisite triazolopyridine bicycle of the compound of formula I, R^{a/b} represents R^{a} or R^{b}, and the other integers (e.g. E², E³, Q¹, J² and E⁴; which are optional substituents that may be attached to specific atoms, or, may be depicted as ‘floating’, in which case the relevant group is optionally substituted by one or more of those E²/Q¹/J²/E³/E⁴ substituents) are as defined herein. The depiction of a substituent in brackets signifies that that substituent is optionally present, and may therefore be absent (i.e. N-(E³) may signify N-E³ or N—H). Further, alkyl or, particularly, heterocycloalkyl groups that are depicted may be further substituted by oxo (═O) groups. The R^{a} or R^{b} group may be as depicted above and for the avoidance of doubt, may be methyl, ethyl or propyl, all of which are optionally substituted by one or more Q¹ substituents; in such instances, there is preferably one Q¹ substituent located at the terminal position of the relevant alkyl group.

Other R² fragments that may be mentioned include:

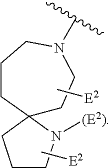

Particularly preferred R² groups include:

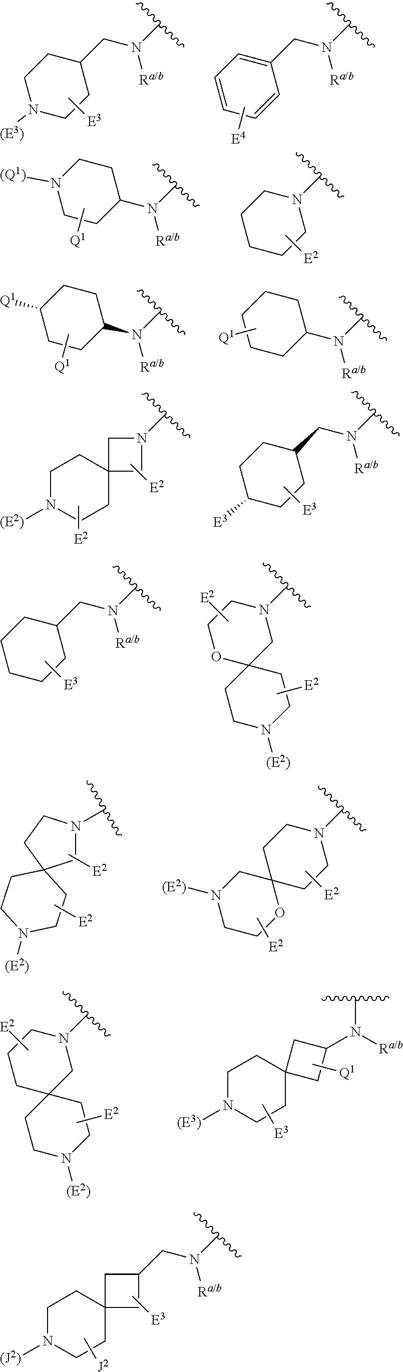

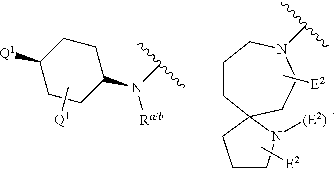

More preferred compounds of the invention that may be mentioned include those in which:

R¹ represents 5- or 6-membered heteroaryl or, especially, aryl (e.g. phenyl) (all of which are) optionally substituted by one to three (e.g. two or, preferably, one) substituent(s) selected from E¹;
one of R^{a} and R^{b} represents hydrogen and the other represents a substituent other than hydrogen;
when R^{a} or R^{b} represents a substituent other than hydrogen, then it is preferably:
(i) acyclic C₁₋₄ (e.g. C₁₋₃) alkyl optionally substituted by one or more (e.g. one) substituent(s) selected from Q¹;
(ii) C₃₋₇ cycloalkyl (e.g. C₄₋₆ cycloalkyl, such as cyclohexyl or cyclobutyl) optionally substituted by one or more (e.g. one) substituent(s) selected from Q¹ (e.g. —OR^{10a} or heterocycloalkyl); or
(iii) heterocycloalkyl (e.g. a 5- or 6-membered monocyclic heterocycloalkyl group, preferably containing one or two heteroatoms preferably selected from nitrogen and oxygen, so forming e.g. tetrahydropyranyl or 4-piperidinyl), which heterocycloalkyl group is optionally substituted by one or more (e.g. one) substituent(s) (which are preferably located on a nitrogen heteroatom, e.g. when on a 4-piperidinyl group) selected from Q¹; or
R^{a} and R^{b} are linked together to form:
a (first) 4- to 7-membered ring (e.g. 4-, 5-, 6- or 7-membered ring, e.g. azetidinyl, pyrrolindinyl, piperidinyl or azepanyl, which may contain a further heteroatom e.g. a nitrogen or oxygen heteroatom, forming e.g. a 4-morpholinyl or piperazinyl group), which ring is optionally substituted by one or more (e.g. one; which is preferably on a nitrogen heteroatom, e.g. in the case of piperazinyl) substituent(s) selected from ═O and, preferably, E², and, further, such a first ring may optionally be either:
(i) fused to another 5- to 7-membered (e.g. 5-membered) heterocycloalkyl group preferably containing one nitrogen heteroatom (e.g. pyrrolidinyl) so forming e.g. a 5,5-fused ring system; or,
(ii) the first ring may be linked via a single atom to another 4- to 6-membered carbocyclic or heterocycloalkyl group (e.g. a 4-, 5- or 6-membered heterocycloalkyl group, preferably containing one or two heteroatoms preferably selected from nitrogen and oxygen, so forming e.g. azetidinyl, pyrrolidinyl, piperidinyl or morpholinyl), to form a spiro cycle (preferred spiro cycles include those in which there are two rings selected from 4-, 5- and 6-membered rings linked together via a single carbon atom common to both rings, e.g. the spiro-cyclic ring system may contain both a 6- and 4-membered ring, a 6- and 5-membered ring, a 6- and 6-membered ring, a 5- and 5-membered ring, or a 7- and 5-membered ring, which are linked to the requisite bicycle of formula I via a nitrogen atom of either of the respective rings; hence [3.5], [4.4], [5.5], [4.5] and [4.6] spiro-cycles are particularly preferred), which second ring of the spiro cycle may be substituted by one or more substituents selected from E² and, if applicable, ═O (forming e.g. an optionally substituted pyrrolidinone);
R³ and R⁴ independently represent hydrogen, —CN, halo or —C(O)OR^{j6} (preferably, at least one of R³ and R⁴ represent hydrogen);
R^{j6} represents hydrogen or preferably C₁₋₄ (e.g. C₁₋₂) alkyl (e.g. ethyl);
Q¹ represents (e.g. when a substituent on an alkyl group, preferably an acyclic alkyl group): —OR^{10a}; —N(R^{10a})R^{11a}; —C(═Y)N(R^{10a})R^{11a}; —N(R^{12a})C(═Y)R^{11a}; aryl (e.g. phenyl) or heteroaryl (e.g. a monocyclic 5- or 6-membered heteroaryl group, preferably containing one or two (e.g. one) heteroatom(s) preferably selected from nitrogen, so forming e.g. a 3-pyridyl group), both of which are optionally substituted by one or more (e.g. one) substituents selected from E⁴; heterocycloalkyl (e.g. a 5- or 6-membered monocyclic heterocycloalkyl group, preferably containing one or two heteroatoms preferably selected from nitrogen and oxygen, so forming e.g. 4-piperidinyl, piperazinyl or imidazolidinyl) optionally substituted by one or more (e.g. one) substituent(s) selected from ═O and E³ (so forming e.g. optionally substituted piperazin-2-ones or imidazolidin-2-ones); or C₃₋₆ cycloalkyl (e.g. cyclopropyl, cyclobutyl or cyclohexyl), which group is may be unsubstituted (e.g. in the case of cyclopropyl) or is optionally substituted by one or more E³ substituents;
Q¹ represents (e.g. when a substituent on a cycloalkyl group) —OR^{10a} or heterocycloalkyl (e.g. a 5- or preferably a 6-membered heterocycloalkyl group, in which there is one heteroatom (preferably selected from nitrogen, e.g. a piperidinyl group), which may be attached via a single carbon atom, to e.g. a cycloalkyl group to which it may be attached, to form a spiro-cyclic structure;
Q¹ represents (e.g. when a substituent on a heterocycloalkyl group, preferably, if the rules of valency allow and if one is present, located on a nitrogen heteroatom) —C(═Y)OR^{10a} or —S(O)₂R^{10a};
R^{10a} and R^{11a} independently represent hydrogen or C₁₋₄ alkyl (e.g. tert-butyl or methyl);
R^{12a} represents hydrogen;
E¹ represents Q⁴ or C₁₋₃ (e.g. C₁₋₂) alkyl (e.g. methyl) optionally substituted by one or more Q⁵ substituents (e.g. in which Q⁵ is fluoro, and hence E¹ may represent a trifluoromethyl group);
E² represents C₁₋₃ (e.g. C₁₋₂) alkyl optionally substituted by one or more (e.g. one) substituent(s) selected from Q⁵, or, E² may represent Q⁴;
E³ represents C₁₋₄ (e.g. C₁₋₃) alkyl (e.g. methyl or cyclopropyl) optionally substituted by one or more (e.g. one) Q⁵ substituent(s) or E³ may also represent Q⁴ (e.g. when located on a heteroatom);
E³ (for instance when attached to a cycloalkyl group) may also represent Q⁴, in which Q⁴ represents heterocycloalkyl (e.g. a 5- or preferably a 6-membered heterocycloalkyl group, in which there is one heteroatom (preferably selected from nitrogen, e.g. a piperidinyl group), which may be attached via a single carbon atom, to e.g. a cycloalkyl group to which it may be attached, to form a spiro-cyclic structure;
E⁴ represents Q⁴;
when E¹ represents Q⁴, then Q⁴ is preferably halo (e.g. fluoro), —OR²⁰ and/or —N(R²²)—C(═Y)—R²¹;
when E² represents Q⁴, then Q⁴ is preferably) —N(R²⁰)R²¹;
when E³ represents Q⁴, then Q⁴ is preferably —OR²⁰, —C(═Y)OR²⁰ or C₁₋₂ alkyl (e.g. methyl) optionally (and preferably) substituted by one J² substituent (or, in this instance, Q⁴ may represent heterocycloalkyl as defined above);
when E⁴ represents Q⁴, then Q⁴ is preferably halo (e.g. fluoro or chloro), —OR²⁰ or —S(O)₂N(R²⁰)R²¹ (e.g. —S(O)₂NH₂);
Q⁵ represents (e.g. when it is a substituent on a E³ alkyl group) halo (e.g. fluoro), —OR²⁰, —C(═Y)N(R²⁰)R²¹, —C(═Y)—R²⁰, heteroaryl (e.g. a 5- or 6-membered monocyclic heteroaryl group, preferably containing one or two heteroatoms, preferably selected from nitrogen, so forming e.g. 4-pyridyl; and which heteroaryl group is preferably unsubstituted) or C₃₋₆ cycloalkyl (e.g. cyclopropyl; preferably unsubstituted);
Q⁵ represents (e.g. when it is a substituent on a E² alkyl group) —N(R²²)C(═Y)R²¹, —OR²⁰ or C₃₋₆ cycloalkyl (e.g. cyclopropyl; preferably unsubstituted) (and, more preferably, Q⁵ represents, when it is a substituent on a E² alkyl group,

### —N(R²²)C(═Y)R²¹);

Y represents ═O;
R²⁰ represents hydrogen or C₁₋₄ alkyl (e.g. methyl, cyclopropyl or tert-butyl) optionally substituted by one or more J⁴ substituents (e.g. in which J⁴ is fluoro, and hence R²⁰ may represent a trifluoromethyl group);
R²¹ represents hydrogen or C₁₋₃ alkyl (e.g. methyl);
R²² represents hydrogen;
J⁴ represents Q⁷;
more preferably, J¹ to J⁶ independently represent Q⁷;
when J² represents Q⁷, then Q⁷ preferably represents —C(═Y^{a})N(R⁵⁰)R⁵¹ or —C(═Y^{a})—R⁵⁰;
when J⁴ represents Q⁷, then Q⁷ preferably represents halo (e.g. fluoro);
Y^{a} represents ═O;
R⁵⁰ and R⁵¹ independently represent C₁₋₄ (e.g. C₁₋₃) alkyl (e.g. methyl or cyclopropyl).

More preferred compounds of the invention that may be mentioned include those in which:

R¹ represents 5- or 6-membered heteroaryl (e.g. pyridyl, such as 3-pyridyl) or aryl (e.g. phenyl) (all of which are) optionally substituted by one to three (e.g. two or, preferably, one) substituent(s) selected from E¹;
R³ and R⁴ independently represent hydrogen, —CN, halo (e.g. chloro or bromo), —OR^{j2} or —C(O)OR^{j6} (preferably, at least one of R³ and R⁴ represent hydrogen);
R⁴ may represent —OR^{j2};
R^{j2} represents C₁₋₃ (e.g. C₁₋₂) alkyl (e.g. methyl);
R³ represents hydrogen, —CN, halo (e.g. chloro or bromo) or —C(O)OR^{j6};
R⁴ represents hydrogen, —CN, halo (e.g. chloro or bromo), —OR^{j2} or —C(O)OR^{j6};
E³ represents C₁₋₄ (e.g. C₁₋₃) alkyl (e.g. isopropyl, isobutyl or preferably methyl or cyclopropyl) optionally substituted by one or more (e.g. one) Q⁵ substituent(s) or
E³ may also represent Q⁴ (e.g. when located on a heteroatom);
E³ (for instance when attached to a cycloalkyl group) may also represent Q⁴, in which Q⁴ represents heterocycloalkyl (e.g. a 5- or preferably a 6-membered heterocycloalkyl group, in which there is one heteroatom (preferably selected from nitrogen, e.g. a piperidinyl group), which may be attached via a single carbon atom, to e.g. a cycloalkyl group to which it may be attached, to form a spiro-cyclic structure (and which heterocycloalkyl group is optionally substituted by one or more (e.g. one) substituent(s) selected from J² (which may be situated on a heteroatom));
when E¹ represents Q⁴, then Q⁴ is preferably halo (e.g. fluoro), —OR²⁰, —N(R²⁰)R²¹ and/or —N(R²²)—C(═Y)—R²¹;
when E² represents Q⁴, then Q⁴ is preferably —OR²⁰, —N(R²⁰)R²¹; —N(R²²)—S(O)₂R²⁰ or —S(O)₂R²⁰;
Q⁵ represents (e.g. when it is a substituent on a E³ alkyl group) halo (e.g. fluoro), —OR²⁰, —C(═Y)N(R²⁰)R²¹, —C(═Y)—R²⁰, heteroaryl (e.g. a 5- or 6-membered monocyclic heteroaryl group, preferably containing one or two heteroatoms, preferably selected from nitrogen, so forming e.g. 4-pyridyl; and which heteroaryl group is preferably unsubstituted), C₃₋₆ cycloalkyl (e.g. cyclopropyl; preferably unsubstituted) or heterocycloalkyl (e.g. a 5- or preferably 6-membered ring, e.g. containing one or two (e.g. one) heteroatom, so forming e.g. tetrahydropyranyl);
J² represents C₁₋₄ alkyl (e.g. C₁₋₂ alkyl, such as ethyl), preferably unsubstituted.

Especially preferred R² groups in the compounds of the invention include those in which:

one of R^{a} and R^{b} represents hydrogen and the other represents a substituent other than hydrogen, or, R^{a} and R^{b} are linked together;
when R^{a} or R^{b} represents a substituent other than hydrogen, then it is preferably:
(i) acyclic C₁₋₄ (e.g. C₁₋₃) alkyl optionally substituted by one or more (e.g. one) substituent(s) selected from Q¹;
(ii) C₃₋₇ cycloalkyl (e.g. C₄₋₆ cycloalkyl, such as cyclohexyl or cyclobutyl) optionally substituted by one or more (e.g. one) substituent(s) selected from Q¹ (e.g. —OR^{10a} or heterocycloalkyl); or
(iii) heterocycloalkyl (e.g. a 5- or, especially, 6-membered monocyclic heterocycloalkyl group, preferably containing one or two heteroatoms preferably selected from nitrogen and oxygen, so forming e.g. 4-piperidinyl), which heterocycloalkyl group is optionally substituted by one or more (e.g. one) substituent(s) selected from Q¹;
when R^{a} and R^{b} are linked together they form:
a (first) 4- to 7-membered ring (e.g. 5-, 6- or 7-membered ring, which may contain a further heteroatom e.g. a nitrogen or oxygen heteroatom), which ring is optionally substituted by one or more substituent(s) selected from E², and, further, such a first ring may optionally be linked via a single atom to another 5- or, especially 6-membered carbocyclic or, preferably, heterocycloalkyl group, preferably containing one or two heteroatoms preferably selected from nitrogen and oxygen;
Q¹ may represent (e.g. when a substituent on an alkyl group, preferably an acyclic alkyl group): aryl (e.g. phenyl) or heteroaryl (e.g. a monocyclic 5- or 6-membered heteroaryl group, preferably containing one or two (e.g. one) heteroatom(s), both of which are optionally substituted by one or more (e.g. one) substituents selected from E⁴; heterocycloalkyl (e.g. a 5- or 6-membered monocyclic heterocycloalkyl group, preferably containing one or two heteroatoms, so forming e.g. 4-piperidinyl) optionally substituted by one or more (e.g. one) substituent(s) selected from ═O and E³; or C₃₋₆ cycloalkyl, which group is may be unsubstituted (e.g. in the case of cyclopropyl) or is optionally substituted by one or more E³ substituents;
Q¹ may represent (e.g. when a substituent on a cycloalkyl group) —OR^{10a} or heterocycloalkyl (e.g. a 5- or preferably a 6-membered heterocycloalkyl group, in which there is one heteroatom (preferably selected from nitrogen, e.g. a piperidinyl group), which may be attached via a single carbon atom, to e.g. a cycloalkyl group to which it may be attached, to form a spiro-cyclic structure;
E³ (for instance when attached to a cycloalkyl group) may represent Q⁴, in which Q⁴ represents heterocycloalkyl (e.g. a 5- or preferably a 6-membered heterocycloalkyl group, in which there is one heteroatom (preferably selected from nitrogen, e.g. a piperidinyl group), which may be attached via a single carbon atom, to e.g. a cycloalkyl group to which it may be attached, to form a spiro-cyclic structure (and which heterocycloalkyl group is optionally substituted by one or more (e.g. one) substituent(s) selected from J² (which may be situated on a heteroatom)).

Particularly preferred compounds include those in which:

R¹ represents 4-methoxyphenyl, 4-hydroxyphenyl, 3-trifluoromethoxyphenyl, unsubstituted phenyl, 4-halophenyl (e.g. 4-fluorophenyl), 4-N(H)—C(O)CH₃-phenyl and 3-trifluoromethylphenyl;
R² represents 1-azepanyl, 4-morpholinyl, —N(H)—CH₂-[4-fluoro-phenyl], —N(H)—CH₂-[3-chloro-phenyl], —N(H)—CH₂—CH₂-[3-pyridyl], —N(H)-n-propyl, —N(H)-[4-tetrahydropyranyl], —N(H)—CH₂—CH₂-[phenyl], —N(H)—CH₂-[1-methyl-piperidin-4-yl], —N(H)—CH₂—CH₂-[2,3-dimethoxyphenyl], —N(H)—CH₂—CH₂-[4-(—S(O)₂—NH₂)-phenyl], —N(H)—CH₂-[piperidin-4-yl], —N(H)—CH₂-[1-(—CH₂C(O)—N(CH₃)₂)-piperidin-4-yl], —N(H)—CH₂-[1-(—CH₂C(O)-cyclopropyl)-piperidin-4-yl], —N(H)—CH₂-[1-CH₂-(4-pyridyl)-piperidin-4-yl], —N(H)—CH₂-[1-cyclopropyl)-piperidin-4-yl], —N(H)—CH₂-[1-(2-fluoro-ethyl)-piperidin-4-yl], 4-(—CH₂CH₂—N(H)—C(O)H)-piperazinyl, —N(H)—CH₂-[1-(2-methoxy-ethyl)-piperidin-4-yl], —N(H)—CH₂-[1-(CH₂-cyclopropyl)-piperidin-4-yl], —N(H)-[(1-C(O)Ot-butyl)-piperidin-4-yl], —N(H)—CH₂CH₂-[(1-C(O)Ot-butyl)piperazin-4-yl], —N(H)—CH₂—CH₂-[2-oxo-4-piperazinyl], —N(H)—CH₂CH₂-[piperazin-1-yl], —N(H)—CH₂CH₂—OH, —N(H)—CH₂CH₂CH₂—OH, —N(H)—CH₂CH₂—N(CH₃)₂, —N(H)—CH₂—C(O)NH₂, —N(H)—CH₂CH₂—N(H)C(O)CH₃, —N(H)—CH₂—CH₂—C(O)NH₂, 2,7-diaza-spiro[3.5]nonane-7-yl, (4-methylamino)piperidin-1-yl, 3,9-diaza-spiro[5,5]undecane-3-yl, octahydro-pyrrolo[3,4-c]pyrrole-1-yl, —N(H)-[4-hydroxy-cyclohexyl], —N(H)—CH₂—CH₂-(1-piperazin-2-one), —N(H)—CH₂—CH₂-(1-imidazolidin-2-one), 2,7-diaza-spiro[3.5]nonane-2-yl, —N(H)—CH₂CH₂CH₂—N(H)(CH₃), 2,8-diaza-spiro[4.5]decan-3-one-8-yl, —N(H)-[4-piperidinyl], —N(H)-(1-methylsulfonyl-4-piperidinyl), —N(H)—CH₂-(4-hydroxy-cyclohexyl), 1-oxa-4,9-diaza-spiro[5.5]undecane-4-yl, 2,9-diaza-spiro[5.5]undecane-2-yl, 2,8-diaza-spiro[4.5]decane-8-yl, 1-oxa-4,8-diaza-spiro[5.5]undecane-8-yl, 2,8-diaza-spiro[4.5]decane-2-yl, 2,7-diaza-spiro[4.4]nonane-2-yl, 1,8-diaza-spiro[4.6]undecane-8-yl, —N(H)-[7-aza-spiro[3.5]nonane-2-yl] and —N(H)—CH₂-[7-aza-spiro[3.5]nonane-2-yl];
R³ represents hydrogen, —C(O)O-ethyl or halo (e.g. chloro);
R⁴ represents hydrogen.

Other R¹ groups that may be mentioned include 6-amino-3-pyridyl, 3-(dimethylamino)phenyl, 4-trifluoromethyl-2-thiazolyl, and 2-trifluoromethyl-4-pyridyl. Other R³ groups that may be mentioned include halo (e.g. chloro and bromo). Other R⁴ groups that may be mentioned include methoxy.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) reaction of a compound of formula II,

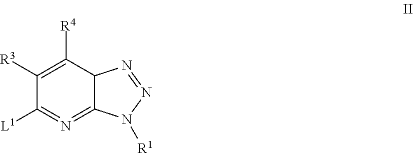

wherein L¹ represents a suitable leaving group, such as iodo, bromo, chloro or a sulfonate group (e.g. —OS(O)₂CF₃, —OS(O)₂CH₃ or —OS(O)₂PhMe) (in particular, L¹ may represent halo, such as chloro or bromo), and R¹, R³ and R⁴ are as hereinbefore defined, with a compound of formula III,

R²—H III

wherein R² is as hereinbefore defined, for example under appropriate coupling reaction conditions, e.g. in the presence of a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof; preferred bases include organic amine bases such as Et₃N) and in the presence of a suitable solvent (such as an alcoholic solvent, e.g. ethanol; other solvents may be employed, such as acetonitrile), which reaction mixture is preferably heated at elevated temperature such as above 50° C. (e.g. at above 80° C., e.g. at about 90° C. or above, e.g. about 150° C.; hence temperatures of between about 60° C. and 170° C. are preferred, depending on the solvent employed). Alternatively, or additionally, microwave irradiation conditions may be employed, for instance to attain the temperatures desired. The reaction may also be performed in the presence of a suitable catalyst system, e.g. a metal (or a salt or complex thereof) such as Pd, CuI, Pd/C, PdCl₂, Pd(OAc)₂, Pd(Ph₃P)₂Cl₂, Pd(Ph₃P)₄ (i.e. palladium tetrakistriphenylphosphine), Pd₂(dba)₃ and/or NiCl₂ (preferred catalysts include palladium) and a ligand such as PdCl₂(dppf).DCM, t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-bi-naphthyl, 1,1′-bis(diphenyl-phosphino-ferrocene), 1,3-bis(diphenyl-phosphino)propane, xantphos, or a mixture thereof (preferred ligands include PdCl₂(dppf).DCM), together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof; preferred bases include Na₂CO₃ and K₂CO₃) in a suitable solvent such as dioxane, toluene, an alcohol (e.g. ethanol), dimethylformamide, dimethoxyethane, ethylene glycol dimethyl ether, water, dimethylsulfoxide, acetonitrile, dimethylacetamide, N-methylpyrrolidinone, tetrahydrofuran or mixtures thereof (preferred solvents include dimethylformamide and dimethoxyethane; pyridine may also be employed, which may serve as the solvent as well as the base). Such reactions may be carried out for example at room temperature or above (e.g. at a high temperature such as at about the reflux temperature of the solvent system). Other reaction conditions that may be mentioned include those in which, especially when L¹ represents tosyl, the reaction may be performed at below room temperature, e.g. at below 0° C., e.g. about −10° C. The reaction may be performed in the presence of an excess of amine of formula III, for instance four or five equivalents thereof;
(ii) for compounds of formula I in which R³ represents halo, reaction of a compound corresponding to a compound of formula I in which R³ represents hydrogen, with a reagent that is a source of halide ions (a halogenating reagent). For instance, an electrophile that provides a source of iodide ions includes iodine, diiodoethane, diiodotetrachloroethane or, preferably, N-iodosuccinimide, a source of bromide ions includes N-bromosuccinimide and bromine, and a source of chloride ions includes N-chlorosuccinimide, chlorine and iodine monochloride, for instance in the presence of a suitable solvent, such as CHCl₃ or an alcohol (e.g. methanol), optionally in the presence of a suitable base, such as a weak inorganic base, e.g. sodium bicarbonate. Typically, the reaction may be performed by heating at a convenient temperature, either by conventional heating under reflux or under microwave irradiation;
(iii) for compounds of formula I in which R³ and/or R⁴ (preferably either one of R³ or R⁴) represents a substituent other that hydrogen or halo (e.g. bromo, iodo or chloro), reaction of a corresponding compound of formula I, in which R³ and/or R⁴ represents halo (e.g. bromo, chloro or iodo), with a compound of formula IV (or two different compounds of formula IV),

R^{3a}-L² IV

wherein R^{3a} represents R³ and/or R⁴ as hereinbefore defined provided that it does not represent hydrogen or halo (and R³ and/or R⁴ preferably represents R^{j1}), and L² represents hydrogen (e.g. in the case where R^{3a} represents —OR^{j2}, —SR^{j3}, —N(R^{j4})R^{j5} or —CN) or a suitable leaving group such as one hereinbefore described in respect of L¹ (e.g. in the case where R^{3a} represents R^{j1}), under reaction conditions known to those skilled in the art, for instance, when L² represents hydrogen, under reaction conditions such as those mentioned hereinbefore; see process step (i) above) or, e.g. in the case where L² represents a leaving group, then reaction may be performed optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, tris(dibenzylideneacetone)-dipalladium(0) (Pd₂(dba)₃) or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). This reaction may be carried out under microwave irradiation reaction conditions or, alternatively, the reaction may be performed in the absence of other reagents such as catalyst, base and even solvent;
(iv) reaction of a compound of formula V,

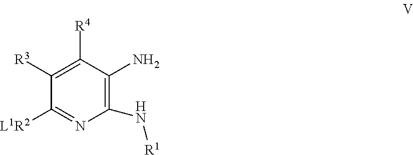

wherein L¹R² represents either L¹ or R³, and R¹, R³ and R⁴ are as hereinbefore defined, with sodium nitrite in the presence of acetic acid and water (for instance the compound of formula V may first be glacial acetic acid, cooled to below room temperature, e.g. about 5° C., after which the sodium nitrite may be added, and the reaction mixture may be allowed to warm to room temperature), or, other similar reagents/conditions that may promote the cyclisation to produce the requisite triazolo-containing bicycle of formula V. In the case where reaction takes place with a compound of formula V in which L¹R² represents either L¹, then the reaction may be proceeded by reaction with a compound of formula III, for example as defined in respect of process step (i) above.

Compounds of formula II may be prepared by reaction of a compound of formula VI,

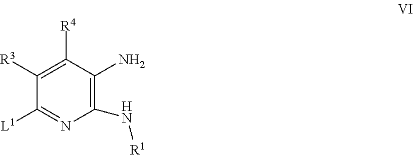

wherein L¹, R¹, R³ and R⁴ are as hereinbefore defined, in the presence of reagents and under reaction conditions such as those hereinbefore described in respect of preparation of compounds of formula I (process step (iv) above).

Compounds of formula II, in which L¹ represents a sulfonate such as a triflate (trimethylsulfonate), may also be prepared by reaction of a corresponding compound of formula VII,

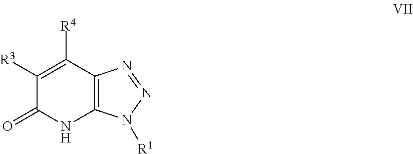

wherein R¹, R³ and R⁴ are as hereinbefore defined, with the relevant sulfonating reagent such as the anhydride (e.g. triflic anhydride), for example in the presence of a suitable base and solvent (e.g. pyridine, which may serve as the solvent and base; alternatively, or additionally, excess of the sulfonating reagent, e.g. triflic anhydride, may be employed to serve as a base). The reaction mixture may be stirred for a period of time, for example at below room temperature, e.g. at about 0° C. or below, e.g. at about −10° C.

Compounds of formula VI may be prepared by hydrogenation of a compound of formula VIII,

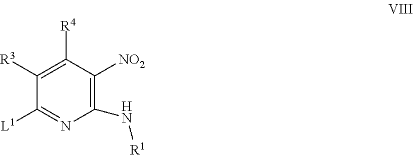

wherein R¹, R³, R⁴ and L¹ are as hereinbefore defined, for example in the presence of suitable hydrogenating conditions, for instance catalytic hydrogenation reaction conditions in the presence of a precious metal catalyst such as Pd or Ni, e.g. raney Ni, and hydrogen (H₂) or a source of hydrogen.

Compounds of formula VII may be prepared by reaction of a compound of formula IX,

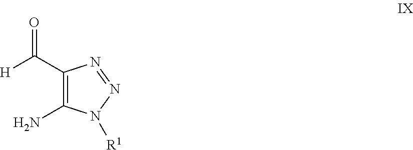

wherein R¹ is as hereinbefore defined, with either a compound of formula X,

(EtO)₂P(O)CH₂—C(O)L³ X

or the like, or a compound of formula XI,

(Ph)₃P═CH—C(O)-L³ XI

or the like, wherein, in each case, L³ represents a suitable leaving group such as one hereinbefore defined (see definition of L¹), and preferably represents —O—C₁₋₆ alkyl (so forming an ester, such as an ethyl ester), followed by intramolecular cyclisation, under standard Horner-Wadsworth-Emmons, or Wittig, reaction conditions, respectively, which reaction may performed in the presence of base e.g. alkali metal based base (e.g. Na₂CO₃, K₂CO₃, K₃PO₄, t-BuONa, t-BuOK or, preferably, sodium alkoxide such as CH₃ONa or EtONa) optionally in the presence of a suitable solvent (e.g. an alcohol, such as ethanol), or mixtures of bases, at elevated temperature and for preferably a prolonged period of time, e.g. over 5 hours (for instance, between 5 and 24 hours).

Compounds of formula VII in which R³ represents —C(O)OC₁₋₆ alkyl (e.g. —C(O)OCH₂CH₃), may be prepared by reaction of a compound of formula IX as hereinbefore defined, with a compound of formula XI,

R^{t2}—O—C(O)—CH₂—C(O)OR^{t1} XI

wherein R^{t1} and R^{t2} independently represent C₁₋₆ alkyl (e.g. ethyl), in the presence of a base such as an alkali metal based base described above (e.g. sodium alkoxide such as CH₃ONa or EtONa) optionally in the presence of a suitable solvent (e.g. an alcohol, such as ethanol), under temperatures and periods of time that may be determined by the skilled person, e.g. at elevated temperature such as at about 55° C. and preferably for prolonged time periods such as over 12 hours.

Compounds of formula VIII may be prepared by reaction of a compound of formula XII,

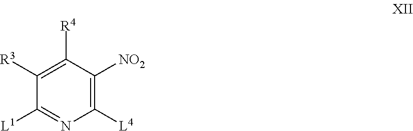

wherein L⁴ represents a suitable leaving groups such as one hereinbefore defined by L¹, and L¹, R³ and R⁴ are as hereinbefore defined (and L¹ and L⁴ both preferably, and independently, represent halo, e.g. chloro), with a compound of formula XIII,

H₂N—R¹ XIII

wherein R¹ is as hereinbefore defined, under standard nucleophilic aromatic substitution reaction conditions (e.g. in the presence of a suitable solvent such as an alcoholic solvent, e.g. ethanol) and optionally in the presence of a suitable base, such as NaHCO₃ (or another suitable base as hereinbefore defined; see e.g. process step (iii) above for preparing compounds of formula I).

Compounds of formula VIII may also be prepared by reaction of a compound of formula XIV,

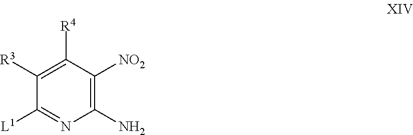

wherein L¹, R³ and R⁴ are as hereinbefore defined, with a compound of formula XV,

R¹-L⁴ XV

wherein R¹ and L⁴ are as hereinbefore defined, for instance L⁴ may represent a suitable leaving group such as halo (e.g. bromo, chloro or iodo) or another suitable leaving group defined under L¹, which reaction may be performed under conditions such as those hereinbefore described in respect of process step (i) above (preparation of compounds of formula I), e.g. in the presence of a suitable catalyst such as Pd(OAc)₂, a suitable ligand such as xantphos and a suitable base such as Cs₂CO₃ (e.g. greater than one equivalent, such as about 2.5 equivalents), which reaction mixture may be in the presence of a polar aprotic solvent (e.g. dioxane), at elevated temperature (e.g. about 110° C.).

Compounds of formula IX may be prepared by standard procedures known to those skilled in the art, for example in accordance with the procedures described in journal article: A. Albert and H. Taguchi, J. Chem. Soc., Perkin I, 1973, 1629.

Compounds of formula XII (for instance in which R⁴ represents —OCH₃) may be prepared by nitration of a corresponding compound of formula XVI,

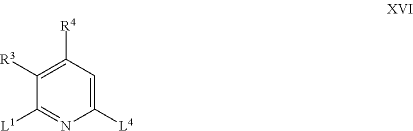

wherein L¹, L⁴, R³ and R⁴ are as hereinbefore defined (and preferably, R⁴ represents —OCH₃) under aromatic nitration reaction conditions, for instance in the presence of a mixture of sulfuric and nitric acid under suitable conditions.

Intermediate and final compounds may be prepared by employing the methods described in journal article “A New General Synthetic Method of [1,2,3]triazolo[4,5-b]pyridines” by Vandendriessche and Weyns (Bull. Soc. Chim. Belg. Vol. 97/no. 1 (1988), pp 85-86).

Other specific transformation steps (including those that may be employed in order to form compounds of formula I) that may be mentioned include:

(i) reductions, for example of a carboxylic acid (or ester) to either an aldehyde or an alcohol, using appropriate reducing conditions (e.g. —C(O)OH (or an ester thereof), may be converted to a —C(O)H or —CH₂—OH group, using DIBAL and LiAlH₄, respectively (or similar chemoselective reducing agents));
(ii) reductions of an aldehyde (—C(O)H) group to an alcohol group (—CH₂OH), using appropriate reduction conditions such as those mentioned at point (i) above;
(iii) oxidations, for example of a moiety containing an alcohol group (e.g. —CH₂OH) to an aldehyde (e.g. —C(O)H), for example in the presence of a suitable oxidising agent, e.g. MnO₂ or the like;
(iv) reductive amination of an aldehyde and an amine, under appropriate reaction conditions, for example in “one-pot” procedure in the presence of an appropriate reducing agent, such as a chemoselective reducing agent such as sodium cyanoborohydride or, preferably, sodium triacetoxyborohydride, or the like. Alternatively, such reactions may be performed in two steps, for example a condensation step (in the presence of e.g. a dehydrating agent such as trimethyl orthoformate or MgSO₄ or molecular sieves, etc) followed by a reduction step (e.g. by reaction in the presence of a reducing agent such as a chemoselective one mentioned above or NaBH₄, AlH₄, or the like), for instance the conversion of —NH₂ to —N(H)-isopropyl by condensation in the presence of acetone (H₃C—C(O)—CH₃) followed by reduction in the presence of a reducing agent such as sodium cyanoborohydride (i.e. overall a reductive amination);
(v) amide coupling reactions, i.e. the formation of an amide from a carboxylic acid (or ester thereof), for example when R² represents —C(O)OH (or an ester thereof), it may be converted to a —C(O)N(R^{10b})R^{11b} group (in which R^{10b} and R^{11b} are as hereinbefore defined, and may be linked together, e.g. as defined above), and which reaction may (e.g. when R² represents —C(O)OH) be performed in the presence of a suitable coupling reagent (e.g. 1,1′-carbonyldiimidazole, N,N′-dicyclohexylcarbodiimide, or the like) or, in the case when R² represents an ester (e.g. —C(O)OCH₃ or —C(O)OCH₂CH₃), in the presence of e.g. trimethylaluminium, or, alternatively the —C(O)OH group may first be activated to the corresponding acyl halide (e.g —C(O)Cl, by treatment with oxalyl chloride, thionyl chloride, phosphorous pentachloride, phosphorous oxychloride, or the like), and, in all cases, the relevant compound is reacted with a compound of formula HN(R^{10a})R^{11a} (in which R^{10a} and R^{11a} are as hereinbefore defined), under standard conditions known to those skilled in the art (e.g. optionally in the presence of a suitable solvent, suitable base and/or in an inert atmosphere);
(vi) conversion of a primary amide to a nitrile functional group, for example under dehydration reaction conditions, e.g. in the presence of POCl₃, or the like;
(vii) nucleophilic substitution reactions, where any nucleophile replaces a leaving group, e.g. methylsulfonylpiperazine may replace a chloro leaving group;
(viii) transformation of a methoxy group to a hydroxy group, by reaction in the presence of an appropriate reagent, such as boron fluoride-dimethyl sulfide complex or BBr₃ (e.g. in the presence of a suitable solvent such as dichloromethane);
(ix) alkylation, acylation or sulfonylation reactions, which may be performed in the presence of base and solvent (such as those described hereinbefore in respect of preparation of compounds of formula I, process step (iv) above, for instance, a —N(H)— or —OH or —NH₂ (or a protected version of the latter) moiety may be alkylated, acylated or sulfonylated by employing a reactant that is an alkyl, acyl or sulfonyl moiety attached to a leaving group (e.g. C₁₋₆ alkyl-halide (e.g. ethylbromide), C₁₋₆ alkyl-C(O)-halide (e.g. H₃C—C(O)Cl), an anhydride (e.g. H₃C—C(O)—O—C(O)—CH₃, i.e. “—O—C(O)—CH₃” is the leaving group), dimethylformamide (i.e. —N(CH₃)₂ is the leaving group) or a sulfonyl halide (e.g. H₃C—S(O)₂Cl) and the like);
(x) specific deprotection steps, such as deprotection of an N-Boc protecting group by reaction in the presence of an acid, or, a hydroxy group protected as a silyl ether (e.g. a tert-butyl-dimethylsilyl protecting group) may be deprotected by reaction with a source of fluoride ions, e.g. by employing the reagent tetrabutylammonium fluoride (TBAF).

Intermediate compounds described herein are either commercially available, are known in the literature, or may be obtained either by analogy with the processes described herein, or by conventional synthetic procedures, in accordance with standard techniques, from available starting materials using appropriate reagents and reaction conditions. Further, processes to prepare compounds of formula I may be described in the literature, for example in:
Werber, G. et al.; J. Heterocycl. Chem.; EN; 14; 1977; 823-827;
Andanappa K. Gadad et al. Bioorg. Med. Chem. 2004, 12, 5651-5659;
Paul Heinz et al. Monatshefte für Chemie, 1977, 108, 665-680;
M. A. El-Sherbeny et al. Boll. Chim. Farm. 1997, 136, 253-256;
Nicolaou, K. C.; Bulger, P. G.; Sarlah, D. Angew. Chem. Int. Ed. 2005, 44, 2-49;
Bretonnet et al. J. Med. Chem. 2007, 50, 1872;
Asunción Marin et al. Farmaco 1992, 47 (1), 63-75;
Severinsen, R. et al. Tetrahedron 2005, 61, 5565-5575;
Nicolaou, K. C.; Bulger, P. G.; Sarlah, D. Angew. Chem. Int. Ed. 2005, 44, 2-49;
M. Kuwahara et al., Chem. Pharm Bull., 1996, 44, 122;
Wipf, P.; Jung, J.-K. J. Org. Chem. 2000, 65(20), 6319-6337;
Shintani, R.; Okamoto, K. Org. Lett. 2005, 7 (21), 4757-4759;
Nicolaou, K. C.; Bulger, P. G.; Sarlah, D. Angew. Chem. Int. Ed. 2005, 44, 2-49;

J. Kobe et al., Tetrahedron, 1968, 24, 239;
P. F. Fabio, A. F. Lanzilotti and S. A. Lang, Journal of Labelled Compounds and Pharmaceuticals, 1978, 15, 407;
F. D. Bellamy and K. Ou, Tetrahedron Lett., 1985, 25, 839;
M. Kuwahara et al., Chem. Pharm Bull., 1996, 44, 122;
A. F. Abdel-Magid and C. A Maryanoff. Synthesis, 1990, 537;
M. Schlosser et al. Organometallics in Synthesis. A Manual, (M. Schlosser, Ed.),
Wiley &Sons Ltd: Chichester, UK, 2002, and references cited therein;
L. Wengwei et al., Tetrahedron Lett., 2006, 47, 1941;
M. Plotkin et al. Tetrahedron Lett., 2000, 41, 2269;
Seyden-Penne, J. Reductions by the Alumino and Borohydrides, VCH, NY, 1991;
O. C. Dermer, Chem. Rev., 1934, 14, 385;
N. Defacqz, et al., Tetrahedron Lett., 2003, 44, 9111;
S. J. Gregson et al., J. Med. Chem., 2004, 47, 1161;
A. M. Abdel Magib, et al., J. Org. Chem., 1996, 61, 3849;
A. F. Abdel-Magid and C. A Maryanoff. Synthesis, 1990, 537;
T. Ikemoto and M. Wakimasu, Heterocycles, 2001, 55, 99;
E. Abignente et al., II Farmaco, 1990, 45, 1075;
T. Ikemoto et al., Tetrahedron, 2000, 56, 7915;
T. W. Greene and P. G. M. Wuts, Protective Groups in Organic Synthesis, Wiley, NY, 1999;
S. Y. Han and Y.-A. Kim. Tetrahedron, 2004, 60, 2447;
J. A. H. Lainton et al., J. Comb. Chem., 2003, 5, 400; or
Wiggins, J. M. Synth. Commun., 1988, 18, 741.

The substituents R¹, R², R³ and R⁴ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence.

For example, when substituents in the compounds of the invention such as CO₂Et, CHO, CN and/or CH₂Cl, are present, these groups can be further derivatized to other fragments described (e.g. by those integers mentioned above) in compounds of the invention, following synthetic protocols very well know to the person skilled in the art and/or according to the experimental part described in the patent. Other specific transformation steps that may be mentioned include: the reduction of a nitro or azido group to an amino group; the hydrolysis of a nitrile group to a carboxylic acid group; and standard nucleophilic aromatic substitution reactions, for example in which an iodo-, preferably, fluoro- or bromo-phenyl group is converted into a cyanophenyl group by employing a source of cyanide ions (e.g. by reaction with a compound which is a source of cyano anions, e.g. sodium, copper (I), zinc or potassium cyanide, optionally in the presence of a palladium catalyst) as a reagent (alternatively, in this case, palladium catalysed cyanation reaction conditions may also be employed).

Other transformations that may be mentioned include: the conversion of a halo group (preferably iodo or bromo) to a 1-alkynyl group (e.g. by reaction with a 1-alkyne), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate nucleophile e.g. a source of the relevant anions, for example by reaction in the presence of a halogen gas (e.g. bromine, iodine or chlorine), or a reagent that is a source of azido or cyanide anions, such as NaN₃ or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Compounds of the invention bearing a carboxyester functional group may be converted into a variety of derivatives according to methods well known in the art to convert carboxyester groups into carboxamides, N-substituted carboxamides, N,N-disubstituted carboxamides, carboxylic acids, and the like. The operative conditions are those widely known in the art and may comprise, for instance in the conversion of a carboxyester group into a carboxamide group, the reaction with ammonia or ammonium hydroxide in the presence of a suitable solvent such as a lower alcohol, dimethylformamide or a mixture thereof; preferably the reaction is carried out with ammonium hydroxide in a methanol/dimethyl-formamide mixture, at a temperature ranging from about 50° C. to about 100° C. Analogous operative conditions apply in the preparation of N-substituted or N,N-disubstituted carboxamides wherein a suitable primary or secondary amine is used in place of ammonia or ammonium hydroxide. Likewise, carboxyester groups may be converted into carboxylic acid derivatives through basic or acidic hydrolysis conditions, widely known in the art. Further, amino derivatives of compounds of the invention may easily be converted into the corresponding carbamate, carboxamido or ureido derivatives.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The need for such protection will vary depending on the nature of the remote functionality and the conditions of the preparation methods (and the need can be readily determined by one skilled in the art). Suitable amino-protecting groups include acetyl, trifluoroacetyl, t-butoxycarbonyl (BOC), benzyloxycarbonyl (CBz), 9-fluorenylmethyleneoxycarbonyl (Fmoc) and 2,4,4-trimethylpentan-2-yl (which may be deprotected by reaction in the presence of an acid, e.g. HCl in water/alcohol (e.g. MeOH)) or the like. The need for such protection is readily determined by one skilled in the art.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined, but without the proviso, for use as a pharmaceutical.

Compounds of the invention may inhibit protein or lipid kinases, such as a PIM family kinase such as PIM-1, PIM-2 and/or PIM-3, and may also inhibit Flt3, for example as may be shown in the tests described below and/or in tests known to the skilled person. Thus, the compounds of the invention may be useful in the treatment of those disorders in an individual in which the inhibition of such protein or lipid kinases (e.g. a PIM family kinase, such as PIM-1, PIM-2 and/or PIM-3, and/or Flt3) is desired and/or required. Advantageously, the compounds of the invention may inhibit both a PIM family kinase and Flt3 (and therefore may act as dual inhibitors).

The term “inhibit” may refer to any measurable reduction and/or prevention of catalytic kinase (e.g. a PIM family kinase, such as PIM-1, PIM-2 and/or PIM-3, and/or Flt3) activity. The reduction and/or prevention of kinase activity may be measured by comparing the kinase activity in a sample containing a compound of the invention and an equivalent sample of kinase (e.g. a PIM family kinase, such as PIM-1, PIM-2 and/or PIM-3, and/or Flt3) in the absence of a compound of the invention, as would be apparent to those skilled in the art. The measurable change may be objective (e.g. measurable by some test or marker, for example in an in vitro or in vivo assay or test, such as one described hereinafter, or otherwise another suitable assay or test known to those skilled in the art) or subjective (e.g. the subject gives an indication of or feels an effect).

Compounds of the invention may be found to exhibit 50% inhibition of a protein or lipid kinase (e.g. a PIM family kinase, such as PIM-1, PIM-2 and/or PIM-3, and/or Flt3) at a concentration of 100 μM or below (for example at a concentration of below 50 μM, or even below 10 μM, such as below 1 μM), when tested in an assay (or other test), for example as described hereinafter, or otherwise another suitable assay or test known to the skilled person.

Compounds of the invention are thus expected to be useful in the treatment of a disorder in which a protein or lipid kinase (e.g. a PIM family kinase, such as PIM-1, PIM-2 and/or PIM-3, and/or Flt3) is known to play a role and which are characterised by or associated with an overall elevated activity of that protein kinase (due to, for example, increased amount of the kinase or increased catalytic activity of the kinase).

Hence, compounds of the invention are expected to be useful in the treatment of a disease/disorder arising from abnormal cell growth, function or behaviour associated with the protein or lipid kinase (e.g. a PIM family kinase, such as PIM-1, PIM-2 and/or PIM-3, and/or Flt3). Such conditions/disorders include cancer, immune disorders, cardiovascular diseases, viral infections, inflammation (e.g. airway inflammation and asthma), metabolism/endocrine function disorders and neurological disorders. In particular, excessive Flt3 activity is associated with refractory AML, so dual inhibitors of a PIM family kinase and Flt3 such as compounds of the invention are useful to treat refractory AML.

The disorders/conditions that the compounds of the invention may be useful in treating hence includes cancer (such as lymphomas, solid tumours or a cancer as described hereinafter), obstructive airways diseases, allergic diseases, inflammatory diseases (such as airway inflammation, asthma, allergy and Chrohn's disease), immunosuppression (such as transplantation rejection and autoimmune diseases), disorders commonly connected with organ transplantation, AIDS-related diseases and other associated diseases. Other associated diseases that may be mentioned (particularly due to the key role of kinases in the regulation of cellular proliferation) include other cell proliferative disorders and/or non-malignant diseases, such as benign prostate hyperplasia, familial adenomatosis, polyposis, neuro-fibromatosis, psoriasis, bone disorders, atherosclerosis, vascular smooth cell proliferation associated with atherosclerosis, pulmonary fibrosis, arthritis glomerulonephritis and post-surgical stenosis and restenosis. Other disease states that may be mentioned include cardiovascular disease, stroke, diabetes, hepatomegaly, Alzheimer's disease, cystic fibrosis, hormone-related diseases, immunodeficiency disorders, destructive bone disorders, infectious diseases, conditions associated with cell death, thrombin-induced platelet aggregation, chronic myelogenous leukaemia, liver disease, pathologic immune conditions involving T cell activation and CNS disorders.

As stated above, the compounds of the invention may be useful in the treatment of cancer. More, specifically, the compounds of the invention may therefore be useful in the treatment of a variety of cancer including, but not limited to: carcinoma such as cancer of the bladder, breast, colon, kidney, liver, lung (including non-small cell cancer and small cell lung cancer), esophagus, gall-bladder, ovary, pancreas, stomach, cervix, thyroid, prostate, skin, squamous cell carcinoma, testis, genitourinary tract, larynx, glioblastoma, neuroblastoma, keratoacanthoma, epidermoid carcinoma, large cell carcinoma, non-small cell lung carcinoma, small cell lung carcinoma, lung adenocarcinoma, bone, adenoma, adenocarcinoma, follicular carcinoma, undifferentiated carcinoma, papilliary carcinoma, seminona, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukaemia; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xeroderma pigmentosum, keratoxanthoma, thyroid follicular cancer and Kaposi's sarcoma,

Further, the protein or lipid kinases (e.g. a PIM family kinase such as PIM-1, PIM-2 and/or PIM-3) may also be implicated in the multiplication of viruses and parasites. They may also play a major role in the pathogenesis and development of neurodegenerative disorders. Hence, compounds of the invention may also be useful in the treatment of viral conditions, parasitic conditions, as well as neurodegenerative disorders.

Compounds of the invention are indicated both in the therapeutic and/or prophylactic treatment of the above-mentioned conditions.

According to a further aspect of the present invention, there is provided a method of treatment of a disease (e.g. cancer or another disease as mentioned herein) which is associated with the inhibition of protein or lipid kinase (e.g. a PIM family kinase, such as PIM-1, PIM-2 and/or PIM-3, and/or Flt3) is desired and/or required (for example, a method of treatment of a disease/disorder arising from abnormal cell growth, function or behaviour associated with protein or lipid kinases, e.g. a PIM family kinase, such as PIM-1, PIM-2 and/or PIM-3, and/or Flt3), which method comprises administration of a therapeutically effective amount of a compound of the invention, as hereinbefore defined, but without the proviso, to a patient suffering from, or susceptible to, such a condition.

“Patients” include mammalian (including human) patients. Hence, the method of treatment discussed above may include the treatment of a human or animal body.

The term “effective amount” refers to an amount of a compound, which confers a therapeutic effect on the treated patient. The effect may be objective (e.g. measurable by some test or marker) or subjective (e.g. the subject gives an indication of or feels an effect).

Compounds of the invention may be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like. The type of pharmaceutical formulation may be selected with due regard to the intended route of administration and standard pharmaceutical practice. Such pharmaceutically acceptable carriers may be chemically inert to the active compounds and may have no detrimental side effects or toxicity under the conditions of use.

Such formulations may be prepared in accordance with standard and/or accepted pharmaceutical practice. Otherwise, the preparation of suitable formulations may be achieved non-inventively by the skilled person using routine techniques and/or in accordance with standard and/or accepted pharmaceutical practice.

According to a further aspect of the invention there is thus provided a pharmaceutical formulation including a compound of the invention, as hereinbefore defined, but without the proviso, in admixture with a pharmaceutically acceptable adjuvant, diluent and/or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The amount of compound of the invention in the formulation will depend on the severity of the condition, and on the patient, to be treated, as well as the compound(s) which is/are employed, but may be determined non-inventively by the skilled person.

The invention further provides a process for the preparation of a pharmaceutical formulation, as hereinbefore defined, which process comprises bringing into association a compound of the invention, as hereinbefore defined, or a pharmaceutically acceptable ester, amide, solvate or salt thereof, but without the proviso, with a pharmaceutically-acceptable adjuvant, diluent or carrier.

Compounds of the invention may also be combined with other therapeutic agents that are inhibitors of protein or lipid kinases (e.g. a PIM family kinase, such as PIM-1, PIM-2 and/or PIM-3, and/or Flt3) and/or useful in the treatment of a cancer and/or a proliferative disease. Compounds of the invention may also be combined with other therapies (e.g. radiation).

For instance, compounds of the invention may be combined with one or more treatments independently selected from surgery, one or more anti-cancer/anti-neoplastic/anti-tumoral agent, one or more hormone therapies, one or more antibodies, one or more immunotherapies, radioactive iodine therapy, and radiation.

More specifically, compounds of the invention may be combined with an agent that modulates the Ras/Raf/Mek pathway (e.g. an inhibitor of MEK), the Jak/Stat pathway (e.g. an inhibitor of Jak), the PI3K/Akt pathway (e.g. an inhibitor of Akt), the DNA damage response mechanism (e.g. an inhibitor of ATM or ATR) or the stress signaling pathway (an inhibitor of p38 or NF-KB).

For instance, compounds of the invention may be combined with:
(i) a targeted kinase inhibitor;
(ii) a receptor tyrosine kinase (RTK) inhibitor;
(iii) an Akt or PI3-K inhibitor, such as GDC-0941;
(iv) an Flt-3 inhibitor;
(v) an EGFR or HER2 inhibitor, such as lapatanib;
(vi) a therapeutic monoclonal antibody, such as the HER2 inhibitor trastuzumab;
(vii) a MEK inhibitor, such as PD-0325901;
(vii) a BRaf inhibitor, such as GDC-0879;
(viii) an anthracyclin, such as doxorubicin;
(ix) a taxane, such as paclitaxel or, particularly, docetaxel (Taxotere);
(x) a platin, such as carboplatin or, particularly, cisplatin;
(xi) a nucleoside analog, such as 5-fluorouracil (5-FU) or gemcitabine);
(xii) an alkylating agent, such as temozolomide;
(xiii) a hormone therapeutic agent, such as an estrogen receptor antagonist e.g. tamoxifen;
(xiv) an anti-tumour compound that has potential radiosensitising and/or chemosensitising effects, such as chloroquine;
(xv) an mTOR inhibitor, such as rapamycin;
(xvi) a JAK inhibitor;
(xvii) a cyclin dependent kinase inhibitor (e.g. a CDK6 or CDK4 inhibitor, such as PD-0332991); and/or
(xviii) an agent that modulates the DNA damage response mechanism and/or the stress signaling pathway, e.g. an inhibitor of ATM or ATR, an inhibitor of p38 and/or NF-KB.

According to a further aspect of the invention, there is provided a combination product comprising:
(A) a compound of the invention, as hereinbefore defined but without the proviso; and
(B) another therapeutic agent that is useful in the treatment of cancer and/or a proliferative disease,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the proviso, another therapeutic agent that is useful in the treatment of cancer and/or a proliferative disease, and a pharmaceutically-acceptable adjuvant, diluent or carrier; and
(2) a kit of parts comprising components:
(a) a pharmaceutical formulation including a compound of the invention, as hereinbefore defined but without the proviso, in admixture with a pharmaceutically-acceptable adjuvant, diluent or carrier; and
(b) a pharmaceutical formulation including another therapeutic agent that is useful in the treatment of cancer and/or a proliferative disease in admixture with a pharmaceutically-acceptable adjuvant, diluent or carrier,
   which components (a) and (b) are each provided in a form that is suitable for administration in conjunction with the other.

In a particularly preferred aspect of the invention, compounds of the invention may be combined with other therapeutic agents (e.g. chemotherapeutic agents) for use as medicaments (e.g. for use in the treatment of a disease or condition as mentioned herein, such as one in which the inhibition of growth of cancer cells are required and/or desired e.g. for treating hyperproliferative disorders such as cancer (e.g. specific cancers that may be mentioned herein, e.g. in the examples) in mammals, especially humans). Such active ingredients in combinations may act in synergy.

In particular, compounds of the invention may be combined with known chemotherapeutic agents (as may be demonstrated by the examples, for instance where a compound of the examples is employed in combination and inhibits cellular proliferative in vitro), for instance:
(i) a PI3K inhibitor, such as GDC-0941;
(ii) an EGFR inhibitor, such as Lapatinib;
(iii) a BRaf inhibitor such as GDC-0879;
(iv) docetaxel (Taxotere®, Sanofi-Aventis);
(v) a MEK inhibitor, such as PD-0325901;
(vi) a CDK4 inhibitor, such as PD-0332991; and/or
(vii) a nucleoside analog, such as Gemcitabine (GEMZAR®, Lilly).

The MEK inhibitor PD-0325901 (CAS RN 391210-10-9, Pfizer) is a second-generation, non-ATP competitive, allosteric MEK inhibitor for the potential oral tablet treatment of cancer (U.S. Pat. No. 6,960,614; U.S. Pat. No. 6,972,298; US 2004/1147478; US 2005/085550). Phase II clinical trials have been conducted for the potential treatment of breast tumors, colon tumors, and melanoma. PD-0325901 is named (R)—N-(2,3-dihydroxypropoxy)-3,4-difluoro-2-(2-fluoro-4-iodophenylamino)benz-amide, and has the structure:

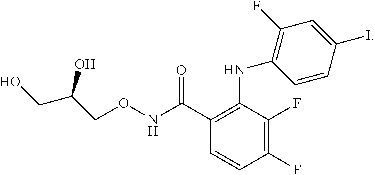 [SMILES: O=C(NOC[C@H](O)CO)c1ccc(F)c(F)c1Nc1ccc(I)cc1F]

Docetaxel (TAXOTERE®, Sanofi-Aventis) is used to treat breast, ovarian, and NSCLC cancers (U.S. Pat. No. 4,814,470; U.S. Pat. No. 5,438,072; U.S. Pat. No. 5,698,582; U.S. Pat. No. 5,714,512; U.S. Pat. No. 5,750,561; Mangatal et at (1989) Tetrahedron 45:4177; Ringel et al (1991) J. Natl. Cancer Inst. 83:288; Bissery et al (1991) Cancer Res. 51:4845; Herbst et al (2003) Cancer Treat. Rev. 29:407-415; Davies et al (2003) Expert. Opin. Pharmacother. 4:553-565). Docetaxel is named as (2R,3S)—N-carboxy-3-phenylisoserine, N-tert-butyl ester, 13-ester with 5, 20-epoxy-1,2,4,7,10,13-hexahydroxytax-11-en-9-one 4-acetate 2-benzoate, trihydrate (U.S. Pat. No. 4,814,470; EP 253738; CAS Reg. No. 114977-28-5) (or named as 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}) and has the structure:

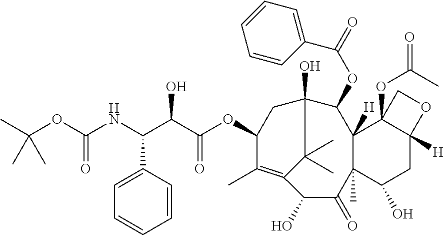 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@](O)([C@@H](OC(=O)c4ccccc4)[C@H]21)C3(C)C]

Lapatinib (TYKERB®, GW572016, Glaxo SmithKline) has been approved for use in combination with capecitabine (XELODA®, Roche) for the treatment of patients with advanced or metastatic breast cancer whose tumors over-express HER2 (ErbB2) and who have received prior therapy including an anthracycline, a taxane and trastuzumab. Lapatinib is an ATP-competitive epidermal growth factor (EGFR) and HER2/neu (ErbB-2) dual tyrosine kinase inhibitor (U.S. Pat. No. 6,727,256; U.S. Pat. No. 6,713,485; U.S. Pat. No. 7,109,333; U.S. Pat. No. 6,933,299; U.S. Pat. No. 7,084,147; U.S. Pat. No. 7,157,466; U.S. Pat. No. 7,141,576) which inhibits receptor autophosphorylation and activation by binding to the ATPbinding pocket of the EGFRIHER2 protein kinase domain. Lapatinib is named as N-(3-chloro-4-(3-fluorobenzyloxy)phenyl)-6-(5-((2-(methylsulfonyl)ethylamino)-methyl)furan-2-yl)quinazolin-4-amine (or alternatively named as N-[3-chloro-4-[(3-fluorophenyl)methoxy]phenyl]-6-[5-[(2-methylsulfonylethylamino)methyl]-2-furyl]quinazolin-4-amine), and has the structure:

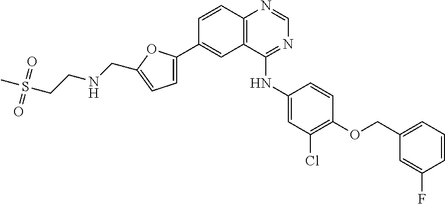 [SMILES: CS(=O)(=O)CCNCc1ccc(-c2ccc3ncnc(Nc4ccc(OCc5cccc(F)c5)c(Cl)c4)c3c2)o1]

Gemcitabine (GEMZAR®, Lilly, CAS Reg. No. 95058-81-4) is a nucleoside analog, which blocks DNA replication, and is used to treat various carcinomas including pancreatic, breast, NSCLC, and lymphomas (U.S. Pat. No. 4,808,614; U.S. Pat. No. 5,464,826; Hertel et at (1988) J. Org. Chem. 53:2406; Hertel et al (1990) Cancer Res. 50:4417; Lund et al (1993) Cancer Treat. Rev. 19:45-55). Gemcitabine is named as 4-amino-1-[3,3-difluoro-4-hydroxy-5-(hydroxymethyl)tetrahydrofuran-2-yl]-1H-pyrimidin-2-one, and has the structure:

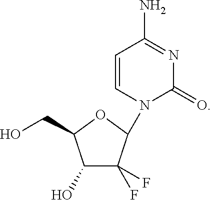 [SMILES: Nc1ccn(C2O[C@H](CO)[C@@H](O)C2(F)F)c(=O)n1]

The invention further provides a process for the preparation of a combination product as hereinbefore defined, which process comprises bringing into association a compound of the invention, as hereinbefore defined, or a pharmaceutically acceptable ester, amide, solvate or salt thereof, but without the proviso, with the other therapeutic agent that is useful in the treatment of cancer and/or a proliferative disease, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Depending on the disorder, and the patient, to be treated, as well as the route of administration, compounds of the invention may be administered at varying therapeutically effective doses to a patient in need thereof. However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of the invention.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

Compounds of the invention may have the advantage that they are effective inhibitors of protein or lipid kinases (e.g. a PIM family kinase, such as PIM-1, PIM-2 and/or PIM-3, and/or Flt3). Advantageously, the compounds of the invention may inhibit both a PIM family kinase and Flt3 (and may therefore be classed as “dual inhibitors”).

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

Compounds of the invention may be beneficial as they are medicaments with targeted therapy, i.e. which target a particular molecular entity by inferring or inhibiting it (e.g. in this case by inhibiting one or more protein or lipid kinases as hereinbefore described). Compounds of the invention may therefore also have the benefit that they have a new effect (for instance as compared to known compounds in the prior art), for instance, the new effect may be a particular mode of action or another effect resultant of the targeted therapy. Targeted therapies may be beneficial as they may have the desired effect (e.g. reduce cancer, by reducing tumor growth or carcinogenisis) but may also have the advantage of reducing side effects (e.g. by preventing the killing of normal cells, as may occur using e.g. chemotherapy).

Furthermore, compounds of the invention may selectively target particular protein or lipid kinases (e.g. the ones described herein) compared to other known protein or lipid kinases (as may be shown experimentally hereinafter; see Table 4 for example). Accordingly, compounds of the invention may have the advantage that certain, specific, cancers may be treated selectively, which selective treatment may also have the effect of reducing side effects.

### EXAMPLES/BIOLOGICAL TESTS

### PIM-1 Biochemical Assay

The biochemical assay to measure PIM-1 activity relies on the ADP Hunter assay kit (DiscoveRx Corp., Cat. #90-0077), that determines the amount of ADP as direct product of the kinase enzyme activity.

The enzyme has been expressed and purified in-house as a recombinant human protein with a C-terminal histidine tag. The protein is active and stable.

Assay conditions were as indicated by the kit manufacturers with the following adaptations for the kinase activity step:
Kinase assay buffer and assay volume stay as recommended (15 mM HEPES, pH 7.4, 20 mM NaCl, 1 mM EGTA, 0.02% Tween 20, 10 mM MgCl₂ and 0.1 mg/ml bovine γ-globulins/75 μl assay volume)
Incubation time and temperature: 60 min at 30° C.
PIM-1 concentration: 50 pg/μl
ATP concentration: 100 μM
PIM-1 substrate peptide: PIMtide (ARKRRRHPSGPPTA)
Peptide concentration: 60 μM
Positive control for kinase activity inhibition: 1-10 μM Staurosporine
DMSO concentration have to stay below 2% during the kinase reaction

Assays were performed in either 96 or 384-well plates. The final outcome of the coupled reactions provided by the kit is the release of the fluorescent product Resorufin and has been measured with a multilabel HTS counter VICTOR V (PerkinElmer) using an excitation filter at 544 nm and an emission filter at 580 nm.

### PIM-2 Biochemical Assay

The biochemical assay to measure PIM-2 activity relies on the ADP Hunter assay kit (DiscoveRx Corp., Cat. #90-0077), that determines the amount of ADP as direct product of the kinase enzyme activity.

The enzyme has been expressed and purified in-house as a recombinant human protein with a N-terminal histidine tag. The protein is active and stable.

Assay conditions were as indicated by the kit manufacturers with the following adaptations for the kinase activity step:
Kinase assay buffer and assay volume stay as recommended (15 mM HEPES, pH 7.4, 20 mM NaCl, 1 mM EGTA, 0.02% Tween 20, 10 mM MgCl₂ and 0.1 mg/ml bovine γ-globulins/20 ml assay volume)
Incubation time and temperature: 30 min at 30° C.
PIM-2 concentration: 350 pg/μl
ATP concentration: 100 μM
PIM-1 substrate peptide: PIMtide (ARKRRRHPSGPPTA)
Peptide concentration: 100 μM
Positive control for kinase activity inhibition: 1-10 μM Staurosporine
DMSO concentration have to stay below 2% during the kinase reaction

Assays were performed in either 96 or 384-well plates. The final outcome of the coupled reactions provided by the kit is the release of the fluorescent product Resorufin and has been measured with a multilabel HTS counter VICTOR V (PerkinElmer) using an excitation filter at 544 nm and an emission filter at 580 nm.

### PIM-3 Biochemical Assay

The biochemical assay to measure PIM-3 activity relies on the ADP Hunter assay kit (DiscoveRx Corp., Cat. #90-0077), that determines the amount of ADP as direct product of the kinase enzyme activity.

The enzyme has been bought from Millipore (#14-738). The protein is active and stable.

Assay conditions were as indicated by the kit manufacturers with the following adaptations for the kinase activity step:
Kinase assay buffer and assay volume stay as recommended (15 mM HEPES, pH 7.4, 20 mM NaCl, 1 mM EGTA, 0.02% Tween 20, 10 mM MgCl₂ and 0.1 mg/ml bovine γ-globulins/20 μl assay volume)
Incubation time and temperature: 30 min at 30° C.
PIM-3 concentration: 250 pg/μl
ATP concentration: 100 μM
PIM-1 substrate peptide: PIMtide (ARKRRRHPSGPPTA)
Peptide concentration: 60 μM
Positive control for kinase activity inhibition: 1-10 μM Staurosporine
DMSO concentration have to stay below 2% during the kinase reaction

Assays were performed in either 96 or 384-well plates. The final outcome of the coupled reactions provided by the kit is the release of the fluorescent product Resorufin and has been measured with a multilabel HTS counter VICTOR V (PerkinElmer) using an excitation filter at 544 nm and an emission filter at 580 nm.

### FLT3 Biochemical Assay

The biochemical assay to measure FLT3 activity relies on the ADP Hunter assay kit (DiscoveRx Corp., Cat. #90-0077 or made in home biochemistry protocol), that determines the amount of ADP as direct product of the kinase enzyme activity.

Assay conditions were as indicated by the kit manufacturers with the following adaptations for the kinase activity step:
Kinase assay buffer and assay volume stay as recommended (15 mM HEPES, pH 7.4, 20 mM NaCl, 1 mM EGTA, 0.02% Tween 20, 10 mM MgCl₂ and 0.1 mg/ml bovine γ-globulins/250 assay volume)
Incubation time and temperature: 60 min at 37° C.
FLT3 final concentration: 0.40 μg/ml
ATP final concentration: 100 μM
ABLtide substrate peptide: EAIYAAPFAKKK
Peptide final concentration: 100 μM
Positive control for kinase activity inhibition:
DMSO concentration have to stay below 2% during the kinase reaction

Assays were performed in either 96 or 384-well plates (corning 3575 or 3573). The final outcome of the coupled reactions provided by the kit (or made in home protocol Bichemistry) is the release of the fluorescent product Resorufin and has been measured with a multilabel HTS counter VICTOR V/ENVISION (PerkinElmer) using an excitation filter at 544 nm and an emission filter at 580 nm. PROTOCOL ENVISION LECTOR PLATE: ADP-HUNTER

### BAD S112 Phosphorilation Inhibition Assay

Efficacy of compounds of the invention on the inhibition of Bad phosphorylation was measured by an In Cell ELISA. EC50 values were established for the tested compounds.

### Assay Conditions:

Cells: H1299 cells overexpressing Pim1 (H1299Pim1)
DMSO Plates: 96-well-Polystyrene, Untreated, Round-Bottom plates from Costar (Cat #3797)
Cell Plates: 96-Flat bottom biocoated with Poly-D-Lysin plates with lid from Becton Dickinson (Cat#354651)
Cell Culture Medium: DMEM high glucose, 10% Fetal Bovine Serum, 2 mM L-Glutamine, P/S
Antibodies: phosphor Bad S112 antibody from Cell Signaling (cat. #9291S), anti rabbit conjugated with peroxidise from Amersham (cat. #3619)
Reagent: SuperSignal ELISA femto from Pierce (cat. #1001110)

### Procedure:

Cells were seeded in 15000 cells per 200 μl per well into 96-well plates and incubated for 16 h at 37° C., 5% CO₂. On day two, nine serial 1:2 compound dilutions were made in DMSO in a 96-well plate. The compounds were added to duplicate wells in 96-well cell plates using a FX BECKMAN robot (Beckman Coulter) and incubated at 37° C. with CO₂ atmosphere. After 4 hours, relative levels of Bad S112 phosphorylation were measured in Cell ELISA using SuperSignal ELISA Femto substrate (Pierce) and read on VICTOR (Perkin Elmer). EC50 values were calculated using ActivityBase from IDBS.

### Combination Assay

Table 5 (see hereinafter) shows the combination index (CI) of combinations of certain example compounds and various chemotherapeutic agents in the MTT in vitro cell proliferation assays. A combination index score is calculated by the Chou and Talalay method (CalcuSyn software, Biosoft). The strength of synergy is scored using the ranking system Chou and Talalay: CI less than 0.8 indicates synergy, CI between 0.8 and 1.2 indicates additivity and CI greater than 1.2 indicates antagonism.

The EC50 values of representative combinations were also calculated. The individually measured EC50 values of the chemotherapeutic agent and the example compounds are compared to the EC50 value of the combination. The cell lines are characterized by tumor type.

Combination assays were performed as described in:

“Pim 1 kinase inhibitor ETP-45299 suppresses cellular proliferation and synergizes with PI3K inhibition” by Blanco-Aparicio, Carmen; Collazo, Ana Maria Garcia; Oyarzabal, Julen; Leal, Juan F.; Albaran, Maria Isabel; Lima, Francisco Ramos; Pequeno, Belen; Ajenjo, Nuria; Becerra, Mercedes; Alfonso, Patricia; Reymundo, Maria Isabel; Palacios, Irene; Mateos, Genoveva; Quinones, Helena; Corrionero, Ana; Carnero, Amancio; Pevarello, Paolo; Lopez, Ana Rodriguez; Fominaya, Jesus; Pastor, Joaquin; Bischoff, James R. Cancer Letters (Shannon, Ireland) 2011, 300(2), 145-153.

### Metabolic Stability Assay and Kinase Selecitivy Testing (See Table 4 Hereinafter)

The metabolic stability assay was performed in Wuxi. 1 μM, 15 min. The kinase selectivity testing was performed in ProQinase GmbH, Germany.

The percentage inhibition of 24 kinases at 1 μM was also determined, in line with procedures known to those skilled in the art (e.g. the procedures may be carried out by ProQuinase). This was to show that the compounds of the invention preferentially (or ‘selectively’) inhibit the kinases mentioned herein (i.e. a PIM family kinase and/or Flt3 and, especially PIM-1 kinase) compared to other kinases.

By preferentially or selectively inhibiting a certain kinase or kinases (e.g. a PIM family kinase and/or Flt3) in favour of another different kinase, we mean that the percentage inhibition at a certain concentration (e.g. at 1 μM) is higher for the favoured kinase or kinases (e.g. a PIM family kinase and/or Flt3, especially PIM-1) than it is for the non-favoured kinase(s). We include that the IC₅₀ values for the favoured kinase or kinases may be lower, as compared to the non-favoured kinase(s).

For instance, compounds of the invention may exhibit ≦30% inhibition of the non-favoured kinases at a concentration of 1 μM, whereas they may exhibit a percentage inhibition of greater than 30% of the favoured kinase(s) (especially PIM-1), for instance ≧50% inhibition at a concentration of 1 μM.

The compound names given herein were generated with MDL ISIS/DRAW 2.5 SP 2, Autonom 2000.

The invention is illustrated by way of the following examples.

### Experimental

The following general schemes may be employed:

### Route 1(a)

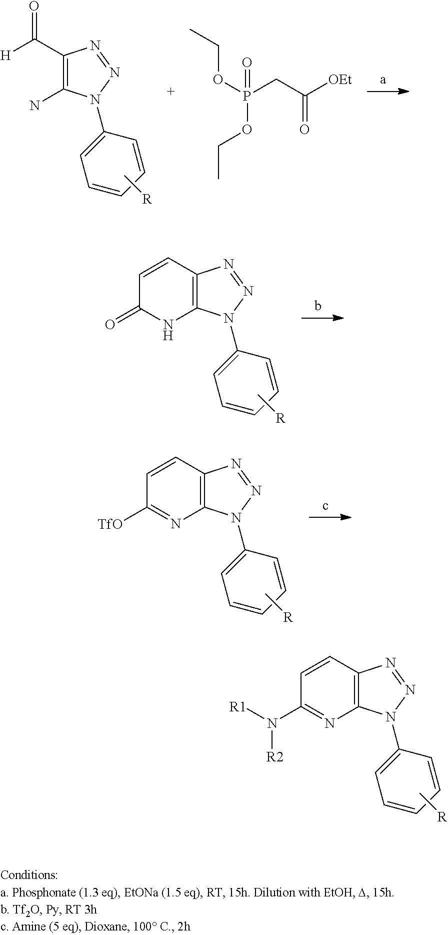

### Route 1(b)

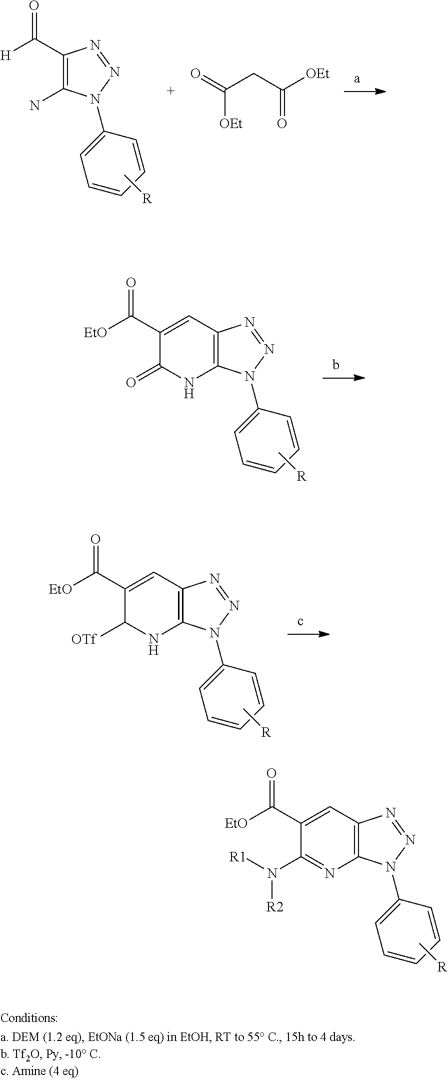

### Route 2(a)

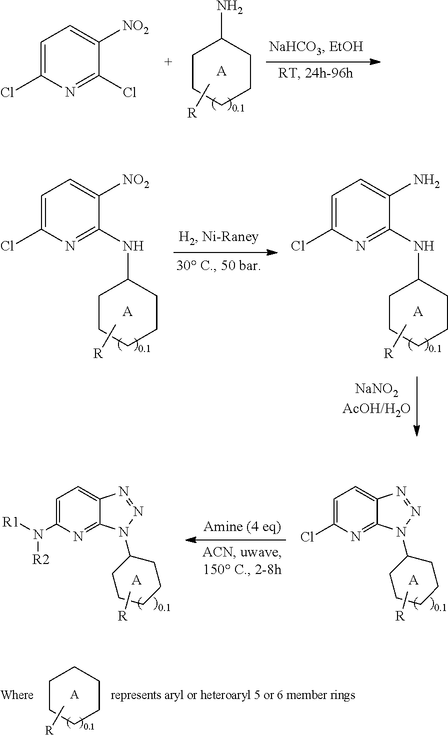

### Route 2(b)

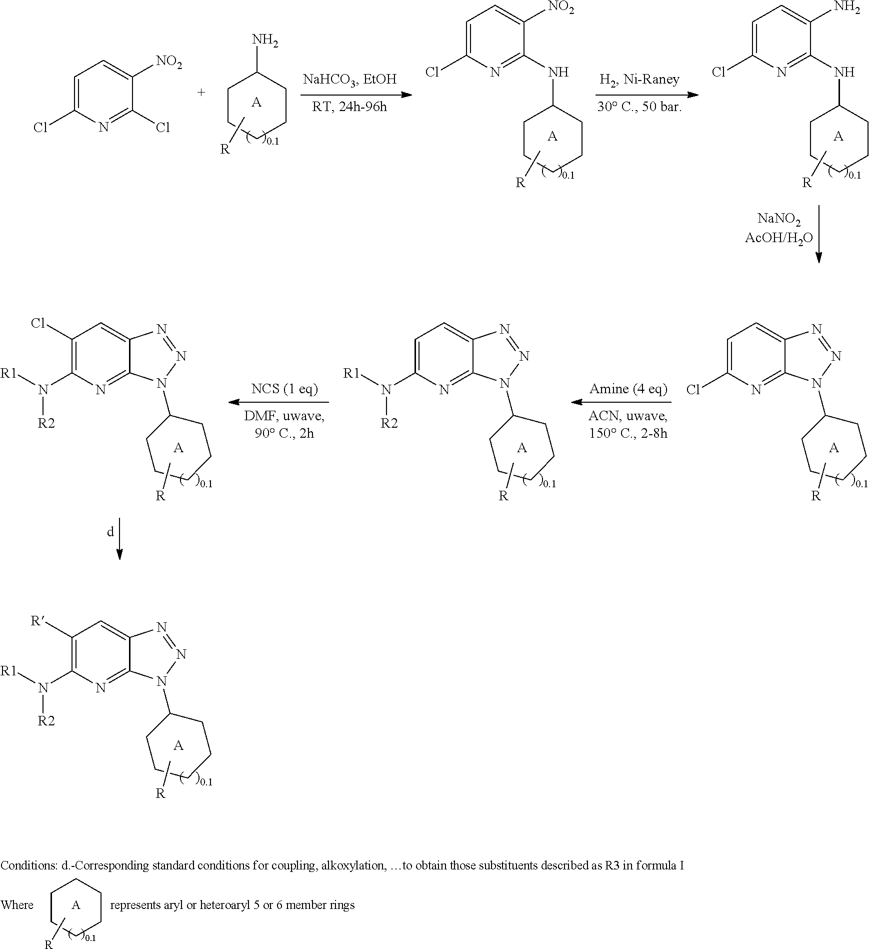

### Route 2(c)

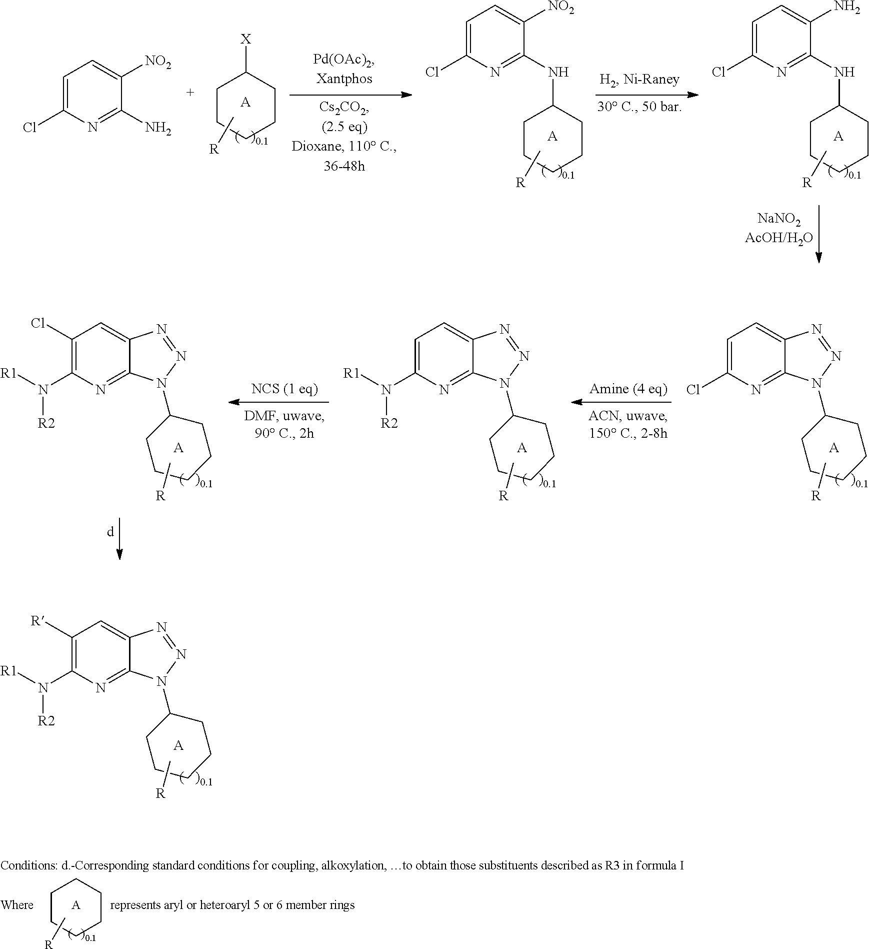

### Route 2(d)

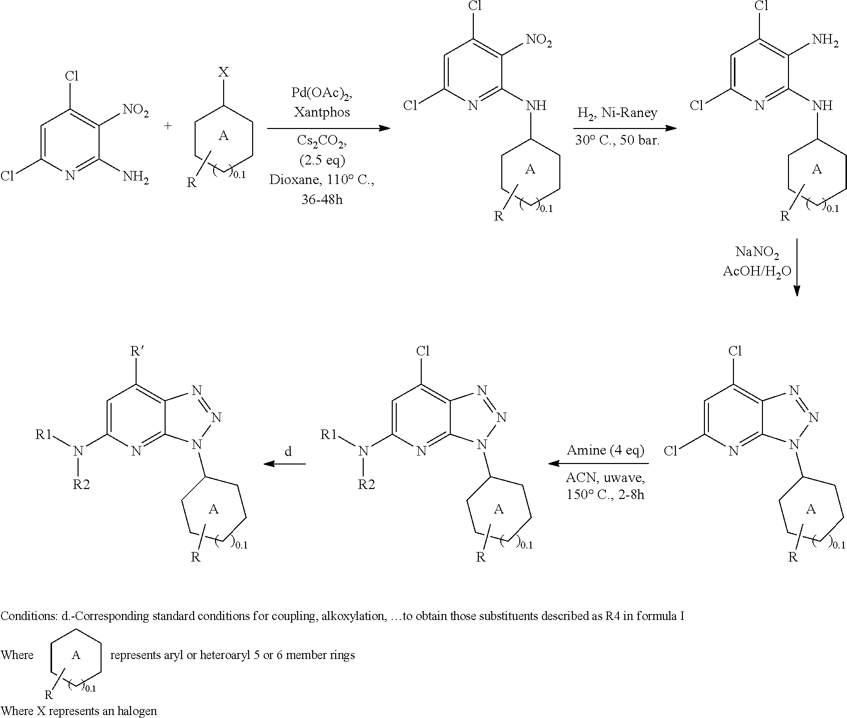

### Route 2 e

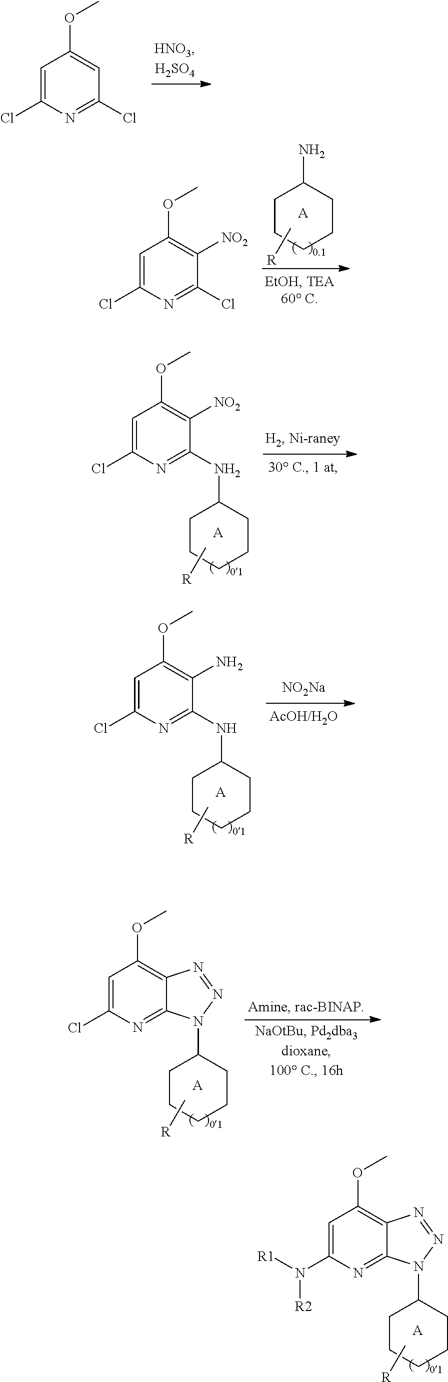

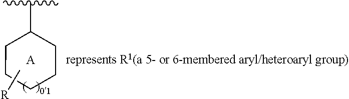

The following terms may be employed herein: “DCM”—dichloromethane, “MeOH”—methanol, “THF”—tetrahydrofuran, “DMF”—dimethylformamide, “DME”—1,2-dimethoxyethane, “EtOAc”—ethyl acetate, “Pd(PPh₃)₄”—tetrakis(triphenyl-phosphine)palladium, “DIPEA”—diisopropylethylamine, “BINAP”—(R)/(+)-2,2′-bis(diphenylphosphino)-1,1′-binaphtyl, “min”—minutes, “h”—hours, “Pd₂(dba)₃”—tris(dibenzylideneacetone)-dipalladium(0), “eq”—equivalents, “nBuOH”—n-butanol, “Pd(dppf)Cl₂.DCM”—1,1′-bis(diphenylphosphino)ferrocenepalladium(II) dichloride, dichloromethane.

### Preparation of Intermediates

### Route 1a

### Preparation of Intermediate I-01

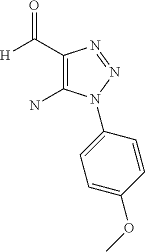 [SMILES: COc1ccc(-n2nnc(C=O)c2N)cc1]

The intermediate I-01, 5-amino-1-(4-methoxy-phenyl)-1H-[1,2,3]triazole-4-carbaldehyde, was explicitly prepared for us by an external CRO following published procedure by A. Albert and H. Taguchi, J. Chem. Soc., Perkin I, 1973, 1629.

Preparation of intermediate I-02

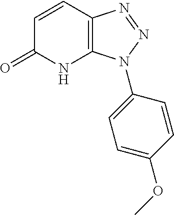 [SMILES: COc1ccc(-n2nnc3ccc(=O)[nH]c32)cc1]

5-Amino-1-(4-methoxy-phenyl)-1H-[1,2,3]triazole-4-carbaldehyde (0.75 g; 3.44 mmol), intermediate I-01, was suspended in dry ethanol (80 mL) and triethyl phosphonoacetate (1.00 g; 4.47 mmol) was added, followed by sodium ethoxide (21% by wt, 3.3 mL; 8.6 mmol). The mixture was heated at 80° C. overnight. The solvent was evaporated and the residue was taken up in DCM and HCl (aq, 0.5N to pH 4). The resulting pale-yellow precipitate was isolated by vacuum filtration and found to correspond to the desired product I-02; 0.72 g were obtained (Y: 86%).

### Preparation of Intermediate I-03

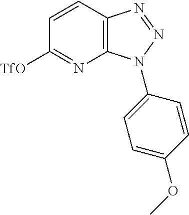 [SMILES: COc1ccc(-n2nnc3ccc(OS(=O)(=O)C(F)(F)F)nc32)cc1]

Triflic anhydride (1.16 g; 4.13 mmol) was added to pyridine (7 mL) at 0° C. and the mixture was stirred for 10 min. The intermediate I-02 (0.50 g; 2.06 mmol) was added and the mixture stirred for 2 h at room temperature.

The solvent was evaporated and the oily residue was taken up in DCM and HCl (aq; 0.1 N). Extraction with DCM, drying and evaporation gave the desired intermediate I-03 as a brown oil in quantitative yield—no additional purification was required to react further.

### Route 1b

### Preparation of Intermediate I-04

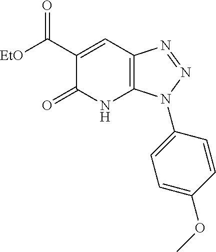 [SMILES: CCOC(=O)c1cc2nnn(-c3ccc(OC)cc3)c2[nH]c1=O]

Diethyl malonate (0.661 g; 4.12 mmol) was dissolved in dry EtOH (50 mL) and NaOEt (1.90 mL; 21% by wt, 5.16 mmol) was added. The mixture was stirred at it for 10 min before adding 5-amino-1-(4-methoxy-phenyl)-1H-[1,2,3]triazole-4-carbaldehyde (0.75 g; 3.44 mmol), intermediate I-01, and the resulting suspension was stirred at 55° C. under Argon over the weekend. Addition of 0.25 eq DEM/EtONa in dry EtOH (2 mL), and reaction continued for another 24 h. The solvent was evaporated and the residue was taken up in water and acidified by add'n of HCl (aq, 2N). The precipitate was isolated by vacuum filtration, washed with ice-cold water and dried by azeotropic distillation with toluene. Corresponds to 3-(4-Methoxy-phenyl)-5-oxo-4,5-dihydro-3H-[1,2,3]triazolo[4,5-b]pyridine-6-carboxylic acid ethyl ester, intermediate I-04; 0.79 g of off-white solid were obtained (Y: 73%).

### Preparation of Intermediate I-05

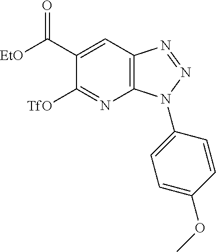 [SMILES: CCOC(=O)c1cc2nnn(-c3ccc(OC)cc3)c2nc1OS(=O)(=O)C(F)(F)F]

Triflic anhydride (0.359 g; 1.273 mmol) was added to dry pyridine (5 mL) at −10° C. The resulting solution was stirred for 10 min before adding the intermediate I-04 (0.200 g; 0.636 mmol). The resulting mixture was stirred for 2 h at the given temperature showed a complete reaction obtaining the desired intermediate I-05. The resulting solution containing the crude triflate intermediate I-05, quantitative yield, was further utilized without additional purification.

### Route 2a

### Preparation of Intermediate I-06

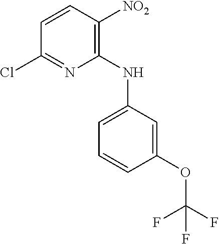 [SMILES: O=[N+]([O-])c1ccc(Cl)nc1Nc1cccc(OC(F)(F)F)c1]

2,6-Dichloro-3-nitropyridine (2.0 g; 10.36 mmol), 3-(trifluoromethoxy)aniline (1.87 g; 10.57 mmol) and sodium hydrogen carbonate (0.87 g; 10.36 mmol) were added to dry ethanol (30 mL). The resulting mixture was stirred for a total of three weeks at room temperature before observing completion of the reaction. The solvent was evaporated and the residue 11074301 was washed with cold ethanol and water to give 2.45 g of the desired product, the intermediate I-06, (6-Chloro-3-nitro-pyridin-2-yl)-(3-trifluoromethoxy-phenyl)-amine as an intensely yellow solid (Y: 71%).

### Preparation of Intermediate I-07

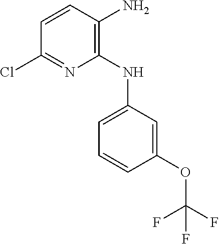 [SMILES: Nc1ccc(Cl)nc1Nc1cccc(OC(F)(F)F)c1]

The nitro compound, intermediate I-06, (0.40 g; 1.199 mmol) was dissolved in ethyl acetate (300 mL) and EtOH (50 mL) and hydrogenated on the H-cube. Conditions utilized for the reduction were as follows, Ni-Raney, 30° C., 50 bar, 1 mL/min, 2 cycles. The reaction was followed by TLC (EtOAc 100%). After 1st cycle: Rf(stm): 0.87 and Rf(prod): 0.43. After 2nd cycle: Only product; the reaction was completed. The solvent was evaporated and the dark-green oily residue slowly crystallized to give 0.32 g of the desired product 6-Chloro-N*2*-(3-trifluoromethoxy-phenyl)-pyridine-2,3-diamine, intermediate I-07 (Y: 88%).

### Preparation of Intermediate I-08

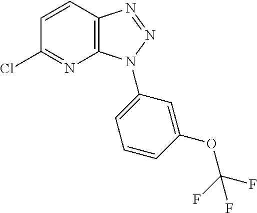 [SMILES: FC(F)(F)Oc1cccc(-n2nnc3ccc(Cl)nc32)c1]

6-Chloro-N*2*-(3-trifluoromethoxy-phenyl)-pyridine-2,3-diamine (0.30 g; 0.99 mmol), intermediate I-07, was dissolved in glacial acetic acid (6 mL) and the solution was cooled to an internal temperature of 5° C. To this solution was added sodium nitrite (0.082 g; 1.19 mmol) and the reaction was allowed to room temperature and stirred for 2 h. The solvent was evaporated and the residue was taken up in water. The crude product was isolated by vacuum filtration as a gummy solid, intermediate I-08, that was further dried by azeotrope distillation with toluene to give 0.305 g of desired product 5-Chloro-3-(3-trifluoromethoxy-phenyl)-3H-[1,2,3]triazolo[4,5-b]pyridine as a beige solid, intermediate I-08 (Y: 98%).

### Route 2(e)

### Intermediate I-10

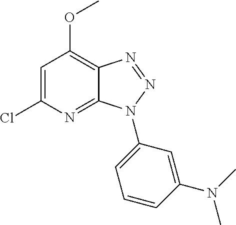 [SMILES: COc1cc(Cl)nc2c1nnn2-c1cccc(N(C)C)c1]

The compound I-11 (558 mg, 1.906 mmol) was dissolved in glacial AcOH (11.2 mL) and the solution was placed in an ice bath. After 5 min, a solution of sodium nitrite (158 mg, 2.287 mmol) in water (1 mL) was added and the reaction was removed from the ice bath. After 30 min stirring, the solvent was evaporated. The resulting residue was purified by automated chromatography in cyclohexane/EtOAc as solvents, to yield expected compound (100 mg, 11% yield)

### Intermediate I-11

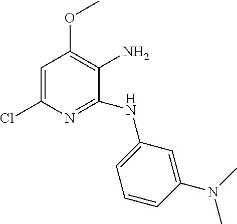 [SMILES: COc1cc(Cl)nc(Nc2cccc(N(C)C)c2)c1N]

4 batches were progressed. Intermediate I-12 (140 mg; 0.434 mmol) was dissolved in EtOH (20 mL). Raney nickel was added as a slurry in water (approximately 2 mL) and the flask was fitted with a rubber septum. After cooling the flask in dry ice for 2 minutes, it was subjected to evacuation-refill cycles with Argon. After the last evacuation cycle, a hydrogen-containing balloon was fitted to the septum. After 5 min., the catalyst was filtered off and the solvent evaporated, to obtain 903 mg of the expected compound.

### Intermediate I-12

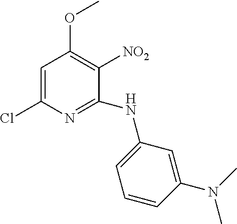 [SMILES: COc1cc(Cl)nc(Nc2cccc(N(C)C)c2)c1[N+](=O)[O-]]

The compound 2,6-Dichloro-4-methoxy-3-nitro-pyridine (488 mg; 2.188 mmol) and N,N-dimethyl-m-phenylenediamine (313 mg; 2.298 mmol) were dissolved in dry EtOH (18 mL) and TEA (305 uL; 2.188 mmol) was added. The reaction was heated at 60° C. for 5 days. The residue was purified in the biotage using a 25M cartridge and cyclohexane/ethyl acetate as solvents. Finally, 1.817 g of the expected compound were obtained (Y.: 64%).

### Intermediate I-13

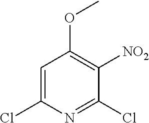

2,6-Dichloro-4-methoxy-pyridine (1.00 g; 5.617 mmol) was dissolved in sulfuric acid (8 mL). The solution was cooled in an ice bath and yellow fuming nitric acid (0.301 mL) was added. The reaction was left at room temp for 30 min, and then heated at 55° C. drysyn temp for 2 h. Excess of reagents were added until reaction was finished. The reaction mixture was poured onto crushed ice, to obtain 1.952 g of the expected compound.

### Preparation of Final Products

### Route 1a

### Preparation of Final Product 2-02

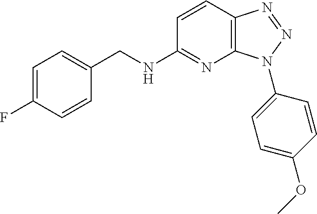 [SMILES: COc1ccc(-n2nnc3ccc(NCc4ccc(F)cc4)nc32)cc1]

Intermediate I-03, trifluoro-methanesulfonic acid 3-(4-methoxy-phenyl)-3H-[1,2,3]triazolo[4,5-b]pyridin-5-yl ester (0.100 g; 0.27 mmol), was weighed into a screwcap vial and dioxane (3 mL) was added. 4-Fluorobenzylamine (0.134 g; 1.07 mmol) was added and the reaction was stirred at 55° C. overnight. The solvent was evaporated and the crude mixture was partitioned between DCM and bicarbonate. Drying and evaporation, followed by purification (Biotage 12S, 10% EtOAc in hexane 5 CV, then ramp to 100% EtOAc over 10 CV) gave 40 mg of the desired final product 2-02, 4-Fluoro-benzyl)-[3-(4-methoxy-phenyl)-3H-[1,2,3]triazolo[4,5-b]pyridin-5-yl]-amine, (Y: 42%).

### Preparation of Final Product 2-04

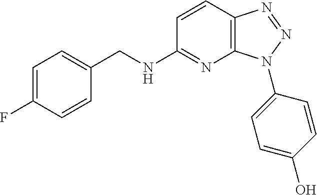 [SMILES: Oc1ccc(-n2nnc3ccc(NCc4ccc(F)cc4)nc32)cc1]

The final compound 2-02, (4-Fluoro-benzyl)-[3-(4-methoxy-phenyl)-3H-[1,2,3]triazolo[4,5-b]pyridin-5-yl]amine (0.040 g; 0.11 mmol), was dissolved in dry DCM (1.5 mL) and boron tribromide solution (0.60 mL; 0.60 mmol; 1M in DCM) was added. The reaction was stirred at room temperature for 4 days. Methanol (1 mL) was added to quench the reaction and after stirring for 1 h, the solvent was evaporated and the residue partitioned between DCM and bicarbonate to give crude product 2-04 as a yellow solid. Further purification was achieved on silica (Manual column; EtOAc:Cyhexane 70:30) and the relevant reactions were evaporated to give 14 mgs of the final product 2-04, 4-[5-(4-Fluoro-benzylamino)-[1,2,3]triazolo[4,5-b]pyridin-3-yl]-phenol, (Y: 37%).

### Route 1b

### Preparation of Final Product 2-14

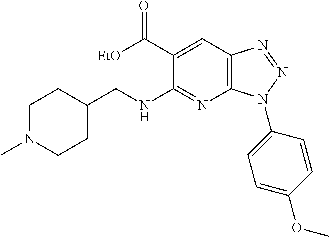

A solution of the intermediate I-05, 3-(4-Methoxy-phenyl)-5-trifluoromethanesulfonyloxy-3H-[1,2,3]triazolo[4,5-b]pyridine-6-carboxylic acid ethyl ester (0.10 g; 0.224 mmol), in dry pyridine (2 mL) was cooled to −10° C. and (1-methyl-4-piperidinyl)methanamine (0.086 g; 0.672 mmol) was added. The resulting solution was stirred for 2 h at this temperature, then allowed to reach room temperature over the weekend. Evaporation of the solvent followed by aqueous workup (DCM/Bicarb drying Na₂SO₄) gave a brown oil that was purified on silica (Biotage 12M; DCM with 5% MeOH 4 CV, gradient to 25% MeOH over 12 CV). The final product 2-14, 3-(4-Methoxy-phenyl)-5-[(1-methyl-piperidin-4-ylmethyl)-amino]-3H-[1,2,3]triazolo[4,5-b]pyridine-6-carboxylic acid ethyl ester, was isolated as an orange solid; in total, 52 mgs were obtained (Y: 55%).

### Route 2a

### Preparation of Final Product 2-10

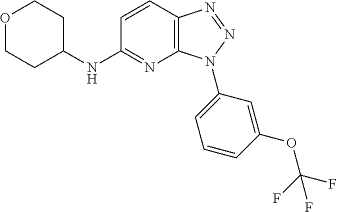 [SMILES: FC(F)(F)Oc1cccc(-n2nnc3ccc(NC4CCOCC4)nc32)c1]

The intermediate I-08, 5-chloro-3-(3-trifluoromethoxy-phenyl)-3H-[1,2,3]triazolo[4,5-b]pyridine (0.075 g; 0.238 mmol), was added to a microwave reaction vessel. Dry acetonitrile (2 mL) was added, followed by 4-aminotetrahydropyran hydrochloride (0.953 mmol, 4 eq) and triethylamine (0.953 mmol). The reaction was heated in the microwave reactor (Biotage) at 150° C. for 4 h. Then, the solvent was evaporated and the reaction was worked up (DCM/Bicarb). The obtained crude product was purified on silica (Biotage 12M, EtOAc/CYhexane. EtOAc 5% 5CV, Gradient to 50% over 12 CV, Gradient to 100% EtOAc over 3 CV, 100% EtOAc 3 CV). Evaporation of the relevant fractions gave the desired final compound 2-10, tetrahydro-pyran-4-yl)-[3-(3-trifluoromethoxy-phenyl)-3H-[1,2,3]triazolo[4,5-b]pyridin-5-yl]-amine; in total, 57 mgs of compound 2-10 were isolated (Y: 54%), as beige-white solid.

### Preparation of Final Product 2-19

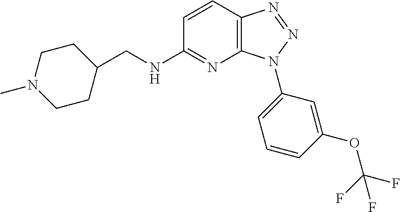 [SMILES: CN1CCC(CNc2ccc3nnn(-c4cccc(OC(F)(F)F)c4)c3n2)CC1]

5-Chloro-3-(3-trifluoromethoxy-phenyl)-3H-[1,2,3]triazolo[4,5-b]pyridine (0.050 g; 0.159 mmol), intermediate I-08, and (1-Methyl-4-piperidinyl)methanamine (0.041 g; 0.318 mmol) were dissolved in dry ethanol (2 mL). Triethylamine (22 uL; 0.159 mmol) was added and the reaction was heated at 90° C. sandbath temp for 72 h. The solvent was evaporated and the crude product was treated with DCM/NaOH (0.1 M). Drying and evaporation gave a yellow solid that was further purified (Biotage 12S. DCM+10% MeOH 5 CV, ramp to 25% MeOH over 10 CV. MeOH with 1% TEA: Thus, the final product 2-19, (1-Methyl-piperidin-4-ylmethyl)-[3-(3-trifluoromethoxy-phenyl)-3H-[1,2,3]triazolo[4,5-b]pyridin-5-yl]-amine, was isolated obtaining 36.9 mgs of a pale-yellow solid (Y: 57%).

### Preparation of Final Product 2-47

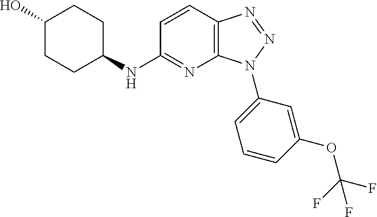 [SMILES: O[C@H]1CC[C@H](Nc2ccc3nnn(-c4cccc(OC(F)(F)F)c4)c3n2)CC1]

The intermediate I-08, 5-chloro-3-(3-trifluoromethoxy-phenyl)-3H-[1,2,3]triazolo[4,5-b]pyridine (0.075 g; 0.238 mmol), was weighted in a vial and trans-4-aminocyclohexanol HCl was added followed by TEA (22 uL; 0.159 mmol) and dry ethanol (2 mL). The reaction was heated at 150° C. sandbath temperature for 36 h. Evaporation of the solvent and extractive workup (DCM/Bicarb) gave the crude compound 2-48 as oily solid. The purification was performed on silica (Biotage 12S; Cyhexane with 10% EtOAc 4 CV, gradient to 100% EtOAc over 15 CV) yielding 22 mgs of final compound 2-47, 1-[3-(3-trifluoromethoxy-phenyl)-3H-[1,2,3]triazolo[4,5-b]pyridin-5-yl]-piperidin-4-ol, as a yellow solid (Y: 36%).

### Preparation of Final Product 2-49

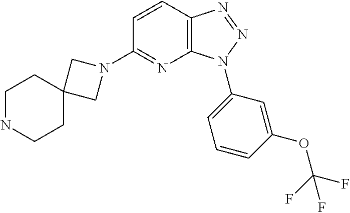 [SMILES: FC(F)(F)Oc1cccc(-n2nnc3ccc(N4CC5(CCNCC5)C4)nc32)c1]

The intermediate I-08, 5-chloro-3-(3-trifluoromethoxy-phenyl)-3H-[1,2,3]triazolo[4,5-b]pyridine (0.050 g; 0.159 mmol), 2,7-diaza-spiro[3.5]nonane-7-carboxylic acid tert-butyl ester, hydrochloride, TEA (66 uL; 0.477 mmol) and dry EtOH (2 mL) were mixed and reacted at 150° C. sandbath temp overnight. Evaporation and extractive workup (DCM/Bicarb) gave the crude boc-protected compound that was pisolated as a yellow precipitate. It was dissolved in dry methanol (2 mL) and HCl (4M in dioxane, 0.25 mL) was added. Overnight stirring at rt, evaporation and free-basing gave 36 mgs of the final compound 2-49, N-methyl-N′-[3-(3-trifluoromethoxy-phenyl)-3H-[1,2,3]triazolo[4,5-b]pyridin-5-yl]-propane-1,3-diamine, as a yellow solid (Y: 46%).

### Route 2b

### Preparation of Final Product 2-55

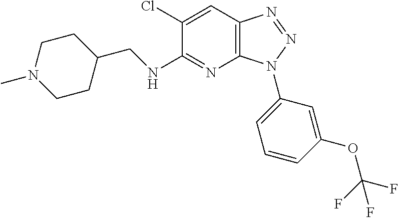 [SMILES: CN1CCC(CNc2nc3c(cc2Cl)nnn3-c2cccc(OC(F)(F)F)c2)CC1]

The final product 2-19, (1-methyl-piperidin-4-ylmethyl)-[3-(3-trifluoromethoxy-phenyl)-3H-[1,2,3]triazolo[4,5-b]pyridin-5-yl]-amine (0.10 g; 0.246 mmol), and N-chlorosuccinimide (0.036 g; 0.271 mmol) were dissolved in dry DMF (2 mL). The reaction was heated at 65° C. for 90 min. whereby another 0.4 eq of NCS was added and heating continued at 75° C. for 1 h. At completed reaction the solvent was evaporated and the residue was purified on silica (Biotage 12S. DCM:MeOH from pure DCM to 30% MeOH over 20 CV. Then further elution with 30% MeOH in DCM with 1% TEA over 15 CV). Evaporation of the relevant fractions gave 11 mg of the final product 2-55, [6-chloro-3-(3-trifluoromethoxy-phenyl)-3H-[1,2,3]triazolo[4,5-b]pyridin-5-yl]-(1-methyl-piperidin-4-ylmethyl)-amine, as a white solid (Y: 10%).

### Route 2e

### Preparation of Final Product 2-93

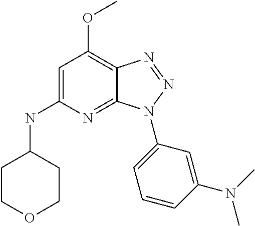 [SMILES: COc1cc(NC2CCOCC2)nc2c1nnn2-c1cccc(N(C)C)c1]

Intermediate I-10 (36 mg; 0.119 mmol), 4-aminotetrahydropyran chloridrate salt (33 mg; 0.237 mmol), rac-BINAP (7 mg; 0.012 mmol), NaOtBu (34 mg; 0.357 mmol) and Pd₂ DBA₃ (5 mg; 0.006 mmol) was dissolved in dry, de-oxygenated dioxane (2 mL) and heated at 100° C. overnight. The solvent was evaporated and the residue was taken up in DCM, bicarbonate was added and it was extracted with DCM. The organic layers were separated, dried over Na₂SO₄, evaporated. The residue was purified in the biotage using a 25M cartridge and cyclohexane/ethyl acetate as solvents. The compound was in the column, so the purification was repeated using the same column, but dichloromethane/methanol as solvents.

Compounds not specifically described were prepared in accordance with the procedures described herein.

### General Procedure

The HPLC measurement was performed using a HP 1100 from Agilent Technologies comprising a pump (binary) with degasser, an autosampler, a column oven, a diode-array detector (DAD) and a column specified in the respective methods below. Flow from the column was split to a MS spectrometer. The MS detector was configured with an electrospray ionization source or API/APCI. Nitrogen was used as the nebulizer gas. The source temperature was maintained at 150° C. Data acquisition was performed with ChemStation LC/MSD quad software.

### Method 1

Reversed phase HPLC was carried out on Gemini-NX C18 (100×2.0 mm; 5 um). Solvent A: water with 0.1% formic acid; Solvent B: acetonitrile with 0.1% formic acid. Gradient: At 50° C., 50% of B to 100% of B within 8 min at 0.6 mL/min; then 100% B at 0.7 mL/min over 2 min, DAD.

### Method 2

Reversed phase HPLC was carried out on Gemini-NX C18 (100×2.0 mm; 5 um). Solvent A: water with 0.1% formic acid; Solvent B: acetonitrile with 0.1% formic acid. Gradient: At 50° C., 5% of B to 100% of B within 8 min at 0.8 mL/min; then 100% B at 0.9 mL/min over 2 min, DAD.

### Method 3

Reversed phase HPLC was carried out on Gemini-NX C18 (100×2.0 mm; 5 um). Solvent A: water with 0.1% formic acid; Solvent B: acetonitrile with 0.1% formic acid. Gradient: At 50° C., 5% of B to 40% of B within 8 min at 0.8 mL/min; then 100% B at 0.9 mL/min over 1 min, DAD.

NMR spectra were recorded in a Bruker Avance II 300 spectrometer and Bruker Avance II 700 spectrometer fitted with 5 mm QXI 700 S4 inverse phase, Z-gradient unit and variable temperature controller.

“Found mass” refers to the most abundant isotope detected in the HPLC-MS.

### EXAMPLES

TABLE 1

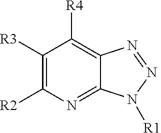

| No. | Exp. | —R1 | —R2 | —R3 | —R4 |
|---|---|---|---|---|---|
| 2-01 | 1a | 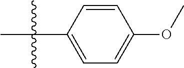 [SMILES: *c1ccc(OC)cc1] | 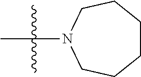 [SMILES: *N1CCCCCC1] | 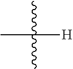 [SMILES: *[H]] | 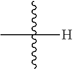 [SMILES: *[H]] |
| 2-02 | 1a | 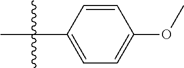 [SMILES: *c1ccc(OC)cc1] | 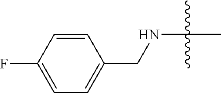 [SMILES: *NCc1ccc(F)cc1] | 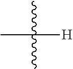 [SMILES: *[H]] | 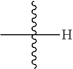 [SMILES: *[H]] |
| 2-03 | 1a | 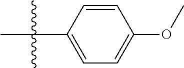 [SMILES: *c1ccc(OC)cc1] | 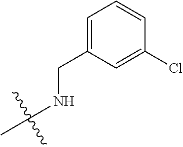 [SMILES: *NCc1cccc(Cl)c1] | 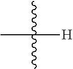 [SMILES: *[H]] | 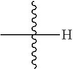 [SMILES: *[H]] |
| 2-04 | 1a | 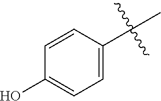 [SMILES: *c1ccc(O)cc1] | 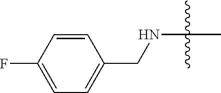 [SMILES: *NCc1ccc(F)cc1] | 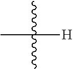 [SMILES: *[H]] | 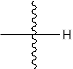 [SMILES: *[H]] |
| 2-05 | 1a | 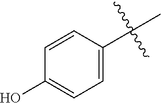 [SMILES: *c1ccc(O)cc1] | 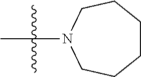 [SMILES: *N1CCCCCC1] | 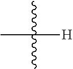 [SMILES: *[H]] | 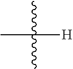 [SMILES: *[H]] |
| 2-06 | 1b | 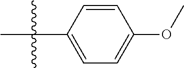 [SMILES: *c1ccc(OC)cc1] | 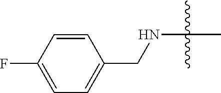 [SMILES: *NCc1ccc(F)cc1] | 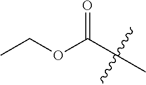 [SMILES: *C(=O)OCC] | 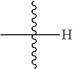 [SMILES: *[H]] |
| 2-07 | 1b | 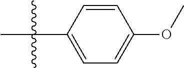 [SMILES: *c1ccc(OC)cc1] | 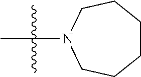 [SMILES: *N1CCCCCC1] | 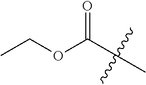 [SMILES: *C(=O)OCC] | 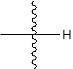 [SMILES: *[H]] |
| 2-08 | 2a | 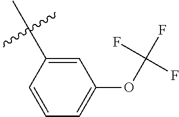 [SMILES: *c1cccc(OC(F)(F)F)c1] | 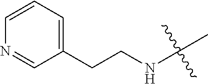 [SMILES: *NCCc1cccnc1] | 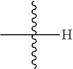 [SMILES: *[H]] | 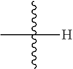 [SMILES: *[H]] |
| 2-09 | 2a | 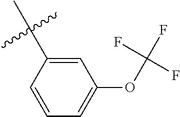 [SMILES: *c1cccc(OC(F)(F)F)c1] | 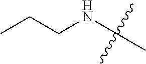 [SMILES: *NCCC] | 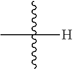 [SMILES: *[H]] | 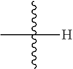 [SMILES: *[H]] |
| 2-10 | 2a | 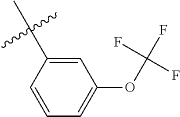 [SMILES: *c1cccc(OC(F)(F)F)c1] | 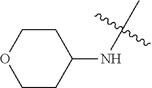 [SMILES: *NC1CCOCC1] | 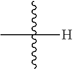 [SMILES: *[H]] | 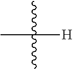 [SMILES: *[H]] |
| 2-11 | 2a | 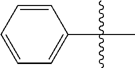 [SMILES: *c1ccccc1] | 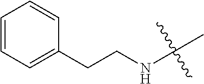 [SMILES: *NCCc1ccccc1] | 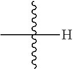 [SMILES: *[H]] | 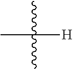 [SMILES: *[H]] |
| 2-12 | 2a | 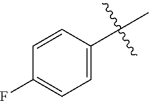 [SMILES: *c1ccc(F)cc1] | 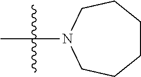 [SMILES: *N1CCCCCC1] | 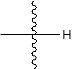 [SMILES: *[H]] | 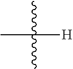 [SMILES: *[H]] |
| 2-13 | 2a | 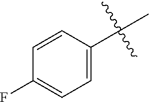 [SMILES: *c1ccc(F)cc1] | 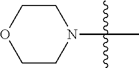 [SMILES: *N1CCOCC1] | 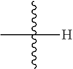 [SMILES: *[H]] | 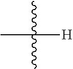 [SMILES: *[H]] |
| 2-14 | 1b | 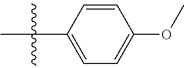 [SMILES: *c1ccc(OC)cc1] | 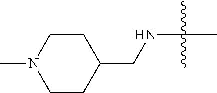 [SMILES: *NCC1CCN(C)CC1] | 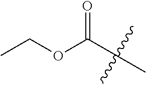 [SMILES: *C(=O)OCC] | 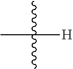 [SMILES: *[H]] |
| 2-15 | 1b | 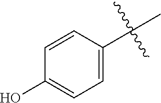 [SMILES: *c1ccc(O)cc1] | 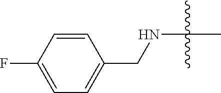 [SMILES: *NCc1ccc(F)cc1] | 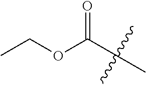 [SMILES: *C(=O)OCC] | 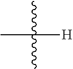 [SMILES: *[H]] |
| 2-16 | 1b | 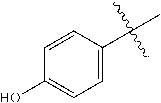 [SMILES: *c1ccc(O)cc1] | 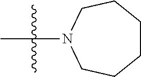 [SMILES: *N1CCCCCC1] | 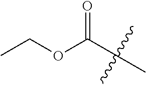 [SMILES: *C(=O)OCC] | 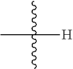 [SMILES: *[H]] |
| 2-17 | 2a | 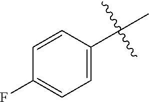 [SMILES: *c1ccc(F)cc1] | 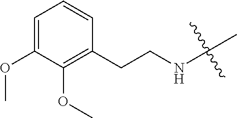 [SMILES: *NCCc1cccc(OC)c1OC] | 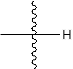 [SMILES: *[H]] | 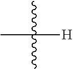 [SMILES: *[H]] |
| 2-18 | 2a | 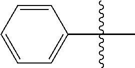 [SMILES: *c1ccccc1] | 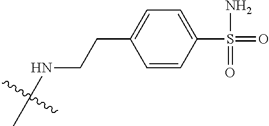 [SMILES: *NCCc1ccc(S(N)(=O)=O)cc1] | 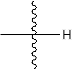 [SMILES: *[H]] | 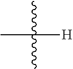 [SMILES: *[H]] |
| 2-19 | 2a | 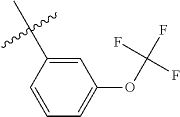 [SMILES: *c1cccc(OC(F)(F)F)c1] | 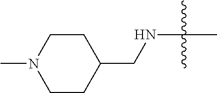 [SMILES: *NCC1CCN(C)CC1] | 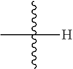 [SMILES: *[H]] | 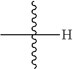 [SMILES: *[H]] |
| 2-20 | 2a | 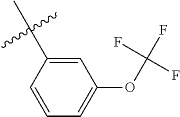 [SMILES: *c1cccc(OC(F)(F)F)c1] | 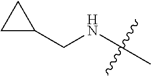 [SMILES: *NCC1CC1] | 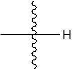 [SMILES: *[H]] | 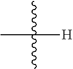 [SMILES: *[H]] |
| 2-21 | 2a | 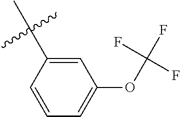 [SMILES: *c1cccc(OC(F)(F)F)c1] | 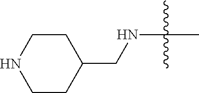 [SMILES: *NCC1CCNCC1] | 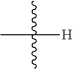 [SMILES: *[H]] | 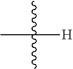 [SMILES: *[H]] |
| 2-22 | 2a | 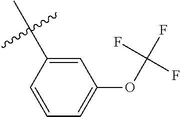 [SMILES: *c1cccc(OC(F)(F)F)c1] | 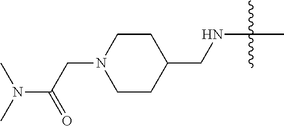 [SMILES: *NCC1CCN(CC(=O)N(C)C)CC1] | 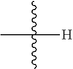 [SMILES: *[H]] | 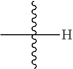 [SMILES: *[H]] |
| 2-23 | 2a | 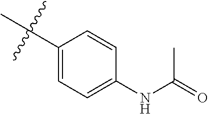 [SMILES: *c1ccc(NC(C)=O)cc1] | 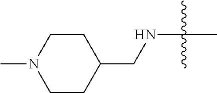 [SMILES: *NCC1CCN(C)CC1] | 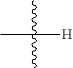 [SMILES: *[H]] | 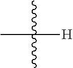 [SMILES: *[H]] |
| 2-24 | 1a | 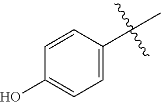 [SMILES: *c1ccc(O)cc1] | 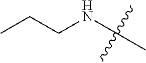 [SMILES: *NCCC] | 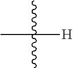 [SMILES: *[H]] | 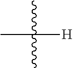 [SMILES: *[H]] |
| 2-25 | 2a | 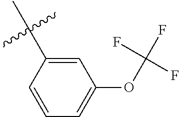 [SMILES: *c1cccc(OC(F)(F)F)c1] | 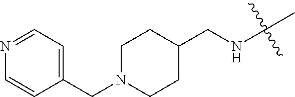 [SMILES: *NCC1CCN(Cc2ccncc2)CC1] | 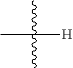 [SMILES: *[H]] | 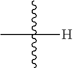 [SMILES: *[H]] |
| 2-26 | 2a | 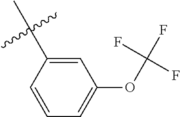 [SMILES: *c1cccc(OC(F)(F)F)c1] | 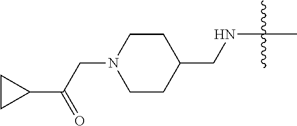 [SMILES: *NCC1CCN(CC(=O)C2CC2)CC1] | 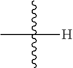 [SMILES: *[H]] | 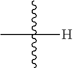 [SMILES: *[H]] |
| 2-27 | 2a | 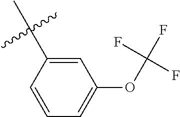 [SMILES: *c1cccc(OC(F)(F)F)c1] | 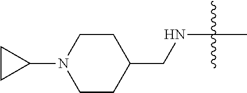 [SMILES: *NCC1CCN(C2CC2)CC1] | 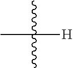 [SMILES: *[H]] | 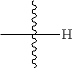 [SMILES: *[H]] |
| 2-28 | 2a | 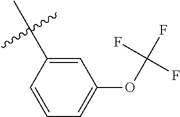 [SMILES: *c1cccc(OC(F)(F)F)c1] | 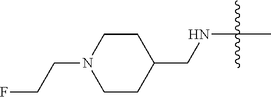 [SMILES: *NCC1CCN(CCF)CC1] | 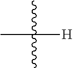 [SMILES: *[H]] | 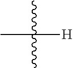 [SMILES: *[H]] |
| 2-29 | 2a | 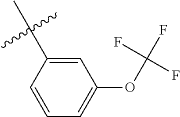 [SMILES: *c1cccc(OC(F)(F)F)c1] | 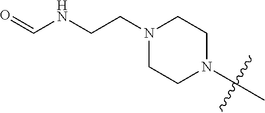 [SMILES: *N1CCN(CCNC=O)CC1] | 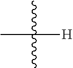 [SMILES: *[H]] | 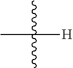 [SMILES: *[H]] |
| 2-30 | 2a | 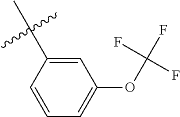 [SMILES: *c1cccc(OC(F)(F)F)c1] | 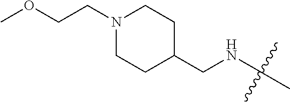 [SMILES: *NCC1CCN(CCOC)CC1] | 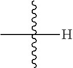 [SMILES: *[H]] | 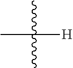 [SMILES: *[H]] |
| 2-31 | 2a | 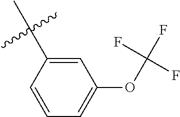 [SMILES: *c1cccc(OC(F)(F)F)c1] | 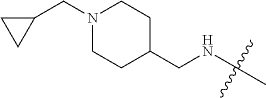 [SMILES: *NCC1CCN(CC2CC2)CC1] | 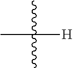 [SMILES: *[H]] | 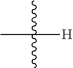 [SMILES: *[H]] |
| 2-32 | 2a | 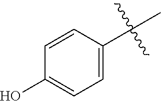 [SMILES: *c1ccc(O)cc1] | 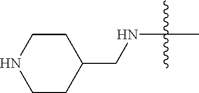 [SMILES: *NCC1CCNCC1] | 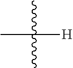 [SMILES: *[H]] | 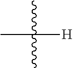 [SMILES: *[H]] |
| 2-33 | 2a | 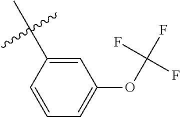 [SMILES: *c1cccc(OC(F)(F)F)c1] | 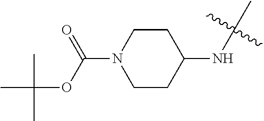 [SMILES: *NC1CCN(C(=O)OC(C)(C)C)CC1] | 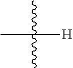 [SMILES: *[H]] | 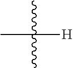 [SMILES: *[H]] |
| 2-34 | 2a | 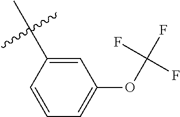 [SMILES: *c1cccc(OC(F)(F)F)c1] | 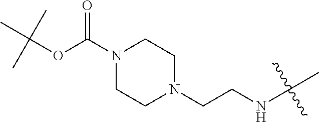 [SMILES: *NCCN1CCN(C(=O)OC(C)(C)C)CC1] | 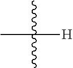 [SMILES: *[H]] | 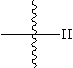 [SMILES: *[H]] |
| 2-35 | 2a | 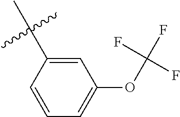 [SMILES: *c1cccc(OC(F)(F)F)c1] | 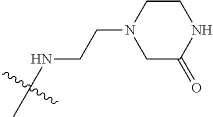 [SMILES: *NCCN1CCNC(=O)C1] | 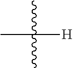 [SMILES: *[H]] | 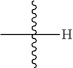 [SMILES: *[H]] |
| 2-36 | 2a | 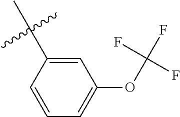 [SMILES: *c1cccc(OC(F)(F)F)c1] | 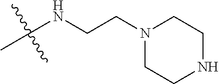 [SMILES: *NCCN1CCNCC1] | 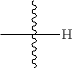 [SMILES: *[H]] | 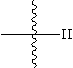 [SMILES: *[H]] |
| 2-37 | 2a | 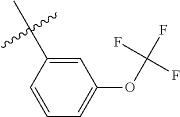 [SMILES: *c1cccc(OC(F)(F)F)c1] | 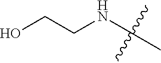 [SMILES: *NCCO] | 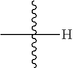 [SMILES: *[H]] | 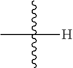 [SMILES: *[H]] |
| 2-38 | 2a | 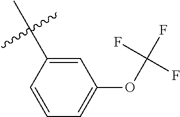 [SMILES: *c1cccc(OC(F)(F)F)c1] | 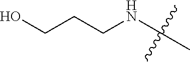 [SMILES: *NCCCO] | 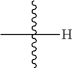 [SMILES: *[H]] | 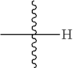 [SMILES: *[H]] |
| 2-39 | 2a | 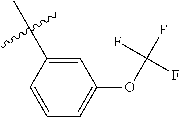 [SMILES: *c1cccc(OC(F)(F)F)c1] | 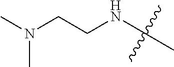 [SMILES: *NCCN(C)C] | 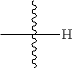 [SMILES: *[H]] | 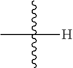 [SMILES: *[H]] |
| 2-40 | 2a | 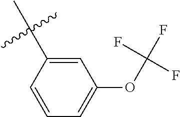 [SMILES: *c1cccc(OC(F)(F)F)c1] | 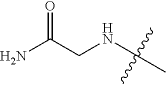 [SMILES: *NCC(N)=O] | 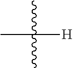 [SMILES: *[H]] | 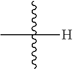 [SMILES: *[H]] |
| 2-41 | 2a | 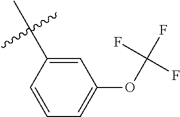 [SMILES: *c1cccc(OC(F)(F)F)c1] | 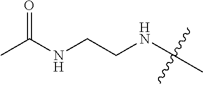 [SMILES: *NCCNC(C)=O] | 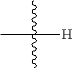 [SMILES: *[H]] | 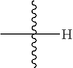 [SMILES: *[H]] |
| 2-42 | 2a | 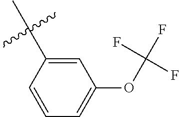 [SMILES: *c1cccc(OC(F)(F)F)c1] | 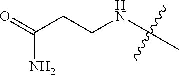 [SMILES: *NCCC(N)=O] | 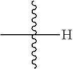 [SMILES: *[H]] | 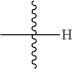 [SMILES: *[H]] |
| 2-43 | 2a | 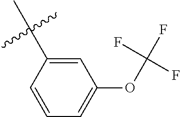 [SMILES: *c1cccc(OC(F)(F)F)c1] | 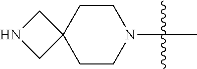 [SMILES: *N1CCC2(CC1)CNC2] | 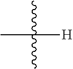 [SMILES: *[H]] | 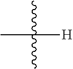 [SMILES: *[H]] |
| 2-44 | 2a | 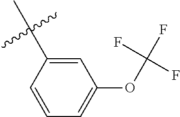 [SMILES: *c1cccc(OC(F)(F)F)c1] | 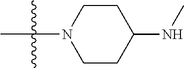 [SMILES: *N1CCC(NC)CC1] | 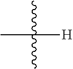 [SMILES: *[H]] | 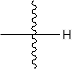 [SMILES: *[H]] |
| 2-45 | 2a | 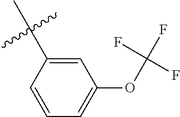 [SMILES: *c1cccc(OC(F)(F)F)c1] | 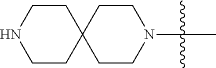 [SMILES: *N1CCC2(CCNCC2)CC1] | 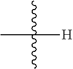 [SMILES: *[H]] | 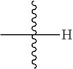 [SMILES: *[H]] |
| 2-46 | 2a | 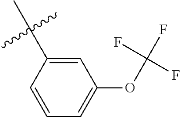 [SMILES: *c1cccc(OC(F)(F)F)c1] | 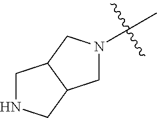 [SMILES: *N1CC2CNCC2C1] | 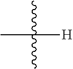 [SMILES: *[H]] | 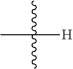 [SMILES: *[H]] |
| 2-47 | 2a | 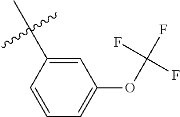 [SMILES: *c1cccc(OC(F)(F)F)c1] | 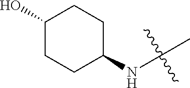 [SMILES: *N[C@H]1CC[C@H](O)CC1] | 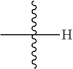 [SMILES: *[H]] | 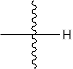 [SMILES: *[H]] |
| 2-48 | 2a | 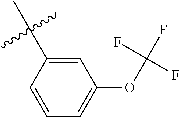 [SMILES: *c1cccc(OC(F)(F)F)c1] | 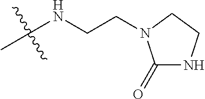 [SMILES: *NCCN1CCNC1=O] | 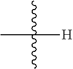 [SMILES: *[H]] | 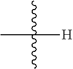 [SMILES: *[H]] |
| 2-49 | 2a | 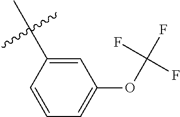 [SMILES: *c1cccc(OC(F)(F)F)c1] | 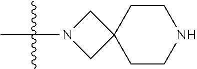 [SMILES: *N1CC2(CCNCC2)C1] | 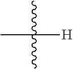 [SMILES: *[H]] | 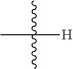 [SMILES: *[H]] |
| 2-50 | 2a | 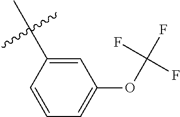 [SMILES: *c1cccc(OC(F)(F)F)c1] | 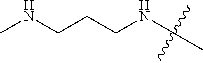 [SMILES: *NCCCNC] | 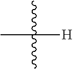 [SMILES: *[H]] | 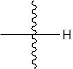 [SMILES: *[H]] |
| 2-51 | 2a | 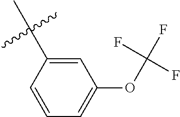 [SMILES: *c1cccc(OC(F)(F)F)c1] | 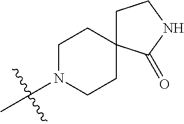 [SMILES: *N1CCC2(CCNC2=O)CC1] | 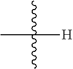 [SMILES: *[H]] | 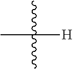 [SMILES: *[H]] |
| 2-52 | 2a | 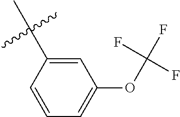 [SMILES: *c1cccc(OC(F)(F)F)c1] | 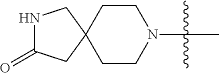 [SMILES: *N1CCC2(CC1)CNC(=O)C2] | 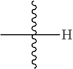 [SMILES: *[H]] | 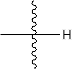 [SMILES: *[H]] |
| 2-53 | 2a | 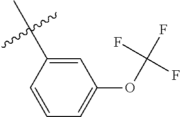 [SMILES: *c1cccc(OC(F)(F)F)c1] | 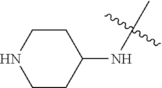 [SMILES: *NC1CCNCC1] | 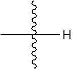 [SMILES: *[H]] | 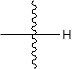 [SMILES: *[H]] |
| 2-54 | 2b | 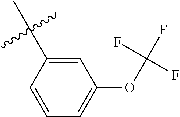 [SMILES: *c1cccc(OC(F)(F)F)c1] | 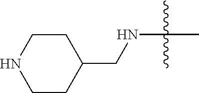 [SMILES: *NCC1CCNCC1] | 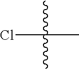 [SMILES: *Cl] | 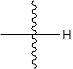 [SMILES: *[H]] |
| 2-55 | 2b | 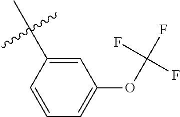 [SMILES: *c1cccc(OC(F)(F)F)c1] | 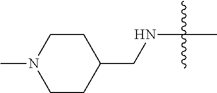 [SMILES: *NCC1CCN(C)CC1] | 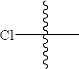 [SMILES: *Cl] | 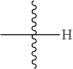 [SMILES: *[H]] |
| 2-56 | 2a | 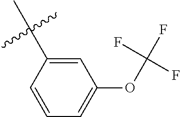 [SMILES: *c1cccc(OC(F)(F)F)c1] | 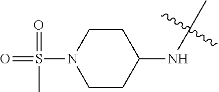 [SMILES: *NC1CCN(S(C)(=O)=O)CC1] | 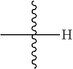 [SMILES: *[H]] | 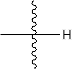 [SMILES: *[H]] |
| 2-57 | 2a | 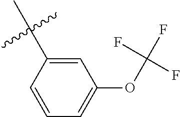 [SMILES: *c1cccc(OC(F)(F)F)c1] | 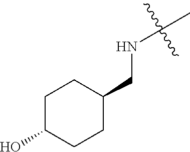 [SMILES: *NC[C@H]1CC[C@H](O)CC1] | 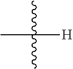 [SMILES: *[H]] | 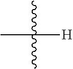 [SMILES: *[H]] |
| 2-58 | 2a | 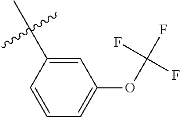 [SMILES: *c1cccc(OC(F)(F)F)c1] | 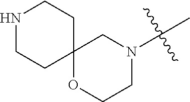 [SMILES: *N1CCOC2(CCNCC2)C1] | 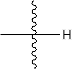 [SMILES: *[H]] | 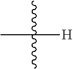 [SMILES: *[H]] |
| 2-59 | 2a | 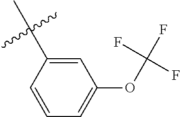 [SMILES: *c1cccc(OC(F)(F)F)c1] | 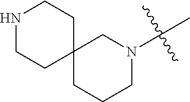 [SMILES: *N1CCCC2(CCNCC2)C1] | 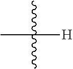 [SMILES: *[H]] | 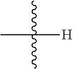 [SMILES: *[H]] |
| 2-60 | 2a | 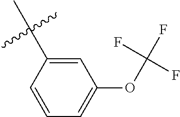 [SMILES: *c1cccc(OC(F)(F)F)c1] | 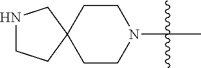 [SMILES: *N1CCC2(CCNC2)CC1] | 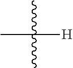 [SMILES: *[H]] | 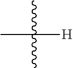 [SMILES: *[H]] |
| 2-61 | 2a | 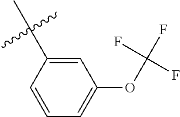 [SMILES: *c1cccc(OC(F)(F)F)c1] | 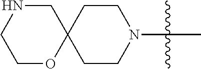 [SMILES: *N1CCC2(CC1)CNCCO2] | 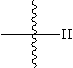 [SMILES: *[H]] | 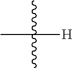 [SMILES: *[H]] |
| 2-62 | 2a | 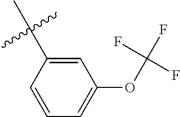 [SMILES: *c1cccc(OC(F)(F)F)c1] | 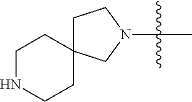 [SMILES: *N1CCC2(CCNCC2)C1] | 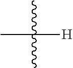 [SMILES: *[H]] | 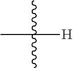 [SMILES: *[H]] |
| 2-63 | 2a | 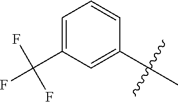 [SMILES: *c1cccc(C(F)(F)F)c1] | 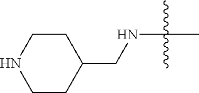 [SMILES: *NCC1CCNCC1] | 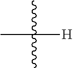 [SMILES: *[H]] | 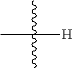 [SMILES: *[H]] |
| 2-64 | 2a | 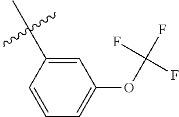 [SMILES: *c1cccc(OC(F)(F)F)c1] | 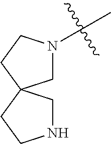 [SMILES: *N1CCC2(CCNC2)C1] | 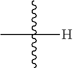 [SMILES: *[H]] | 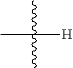 [SMILES: *[H]] |
| 2-65 | 2a | 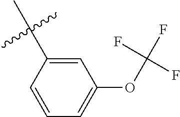 [SMILES: *c1cccc(OC(F)(F)F)c1] | 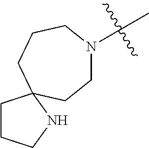 [SMILES: *N1CCCC2(CCCN2)CC1] | 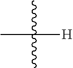 [SMILES: *[H]] | 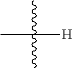 [SMILES: *[H]] |
| 2-66 | 2a | 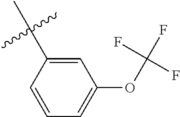 [SMILES: *c1cccc(OC(F)(F)F)c1] | 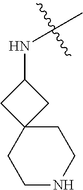 [SMILES: *NC1CC2(CCNCC2)C1] | 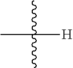 [SMILES: *[H]] | 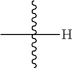 [SMILES: *[H]] |
| 2-67 | 2a | 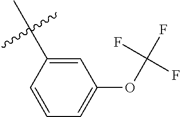 [SMILES: *c1cccc(OC(F)(F)F)c1] | 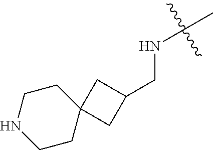 [SMILES: *NCC1CC2(CCNCC2)C1] | 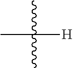 [SMILES: *[H]] | 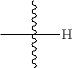 [SMILES: *[H]] |
| 2-68 | 2a | 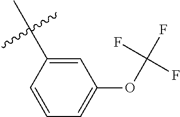 [SMILES: *c1cccc(OC(F)(F)F)c1] | 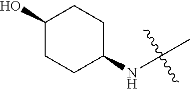 [SMILES: *N[C@H]1CC[C@@H](O)CC1] | 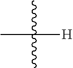 [SMILES: *[H]] | 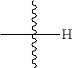 [SMILES: *[H]] |
| 2-69 | 2a | 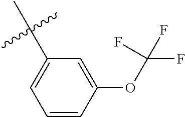 [SMILES: *c1cccc(OC(F)(F)F)c1] | 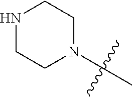 [SMILES: *N1CCNCC1] | 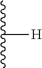 [SMILES: *[H]] | 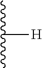 [SMILES: *[H]] |
| 2-70 | 2a | 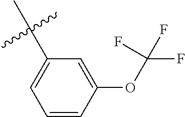 [SMILES: *c1cccc(OC(F)(F)F)c1] | 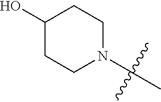 [SMILES: *N1CCC(O)CC1] | 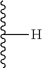 [SMILES: *[H]] | 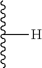 [SMILES: *[H]] |
| 2-71 | 2a | 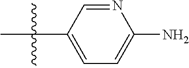 [SMILES: *c1ccc(N)nc1] | 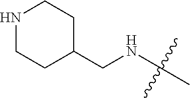 [SMILES: *NCC1CCNCC1] | 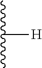 [SMILES: *[H]] | 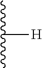 [SMILES: *[H]] |
| 2-72 | 2a | 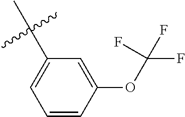 [SMILES: *c1cccc(OC(F)(F)F)c1] | 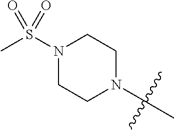 [SMILES: *N1CCN(S(C)(=O)=O)CC1] | 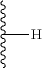 [SMILES: *[H]] | 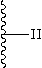 [SMILES: *[H]] |
| 2-73 | 2a | 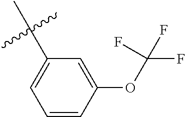 [SMILES: *c1cccc(OC(F)(F)F)c1] | 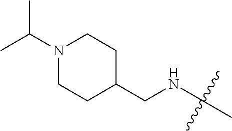 [SMILES: *NCC1CCN(C(C)C)CC1] | 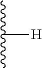 [SMILES: *[H]] | 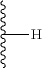 [SMILES: *[H]] |
| 2-74 | 2a | 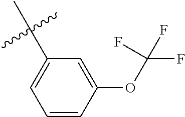 [SMILES: *c1cccc(OC(F)(F)F)c1] | 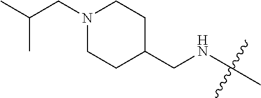 [SMILES: *NCC1CCN(CC(C)C)CC1] | 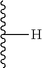 [SMILES: *[H]] | 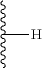 [SMILES: *[H]] |
| 2-75 | 2a | 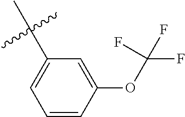 [SMILES: *c1cccc(OC(F)(F)F)c1] | 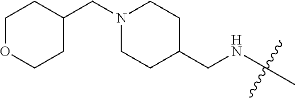 [SMILES: *NCC1CCN(CC2CCOCC2)CC1] | 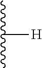 [SMILES: *[H]] | 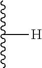 [SMILES: *[H]] |
| 2-76 | 2a | 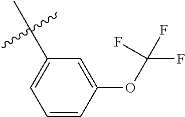 [SMILES: *c1cccc(OC(F)(F)F)c1] | 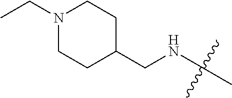 [SMILES: *NCC1CCN(CC)CC1] | 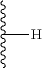 [SMILES: *[H]] | 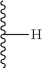 [SMILES: *[H]] |
| 2-77 | 2b | 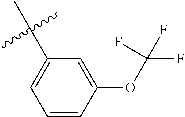 [SMILES: *c1cccc(OC(F)(F)F)c1] | 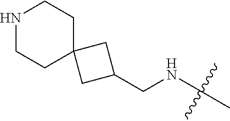 [SMILES: *NCC1CC2(CCNCC2)C1] | 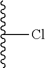 [SMILES: *Cl] | 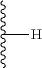 [SMILES: *[H]] |
| 2-78 | 2b | 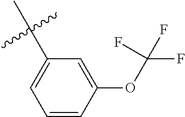 [SMILES: *c1cccc(OC(F)(F)F)c1] | 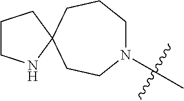 [SMILES: *N1CCCC2(CCCN2)CC1] | 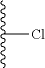 [SMILES: *Cl] | 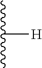 [SMILES: *[H]] |
| 2-79 | 2b | 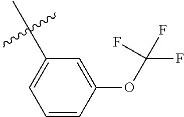 [SMILES: *c1cccc(OC(F)(F)F)c1] | 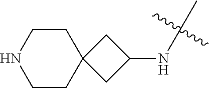 [SMILES: *NC1CC2(CCNCC2)C1] | 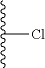 [SMILES: *Cl] | 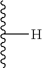 [SMILES: *[H]] |
| 2-80 | 2a | 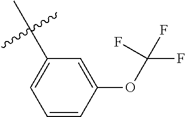 [SMILES: *c1cccc(OC(F)(F)F)c1] | 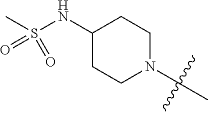 [SMILES: *N1CCC(NS(C)(=O)=O)CC1] | 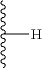 [SMILES: *[H]] | 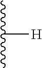 [SMILES: *[H]] |
| 2-81 | 2a | 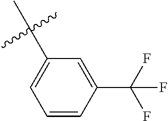 [SMILES: *c1cccc(C(F)(F)F)c1] | 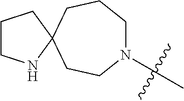 [SMILES: *N1CCCC2(CCCN2)CC1] | 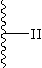 [SMILES: *[H]] | 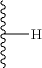 [SMILES: *[H]] |
| 2-82 | 2a | 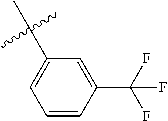 [SMILES: *c1cccc(C(F)(F)F)c1] | 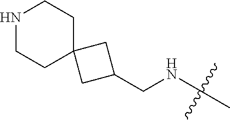 [SMILES: *NCC1CC2(CCNCC2)C1] | 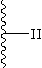 [SMILES: *[H]] | 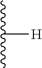 [SMILES: *[H]] |
| 2-83 | 2b | 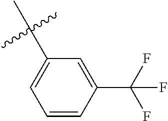 [SMILES: *c1cccc(C(F)(F)F)c1] | 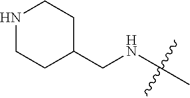 [SMILES: *NCC1CCNCC1] | 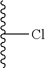 [SMILES: *Cl] | 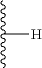 [SMILES: *[H]] |
| 2-84 | 2a | 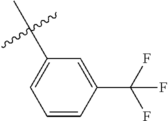 [SMILES: *c1cccc(C(F)(F)F)c1] | 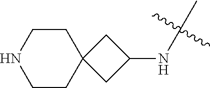 [SMILES: *NC1CC2(CCNCC2)C1] | 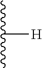 [SMILES: *[H]] | 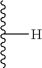 [SMILES: *[H]] |
| 2-85 | 2b | 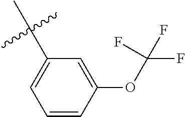 [SMILES: *c1cccc(OC(F)(F)F)c1] | 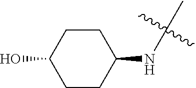 [SMILES: *N[C@H]1CC[C@H](O)CC1] | 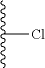 [SMILES: *Cl] | 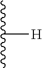 [SMILES: *[H]] |
| 2-86 | 2a | 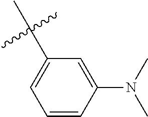 [SMILES: *c1cccc(N(C)C)c1] | 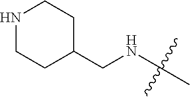 [SMILES: *NCC1CCNCC1] | 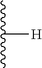 [SMILES: *[H]] | 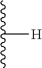 [SMILES: *[H]] |
| 2-87 | 2d or 2e | 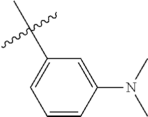 [SMILES: *c1cccc(N(C)C)c1] | 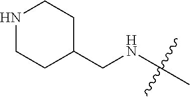 [SMILES: *NCC1CCNCC1] | 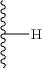 [SMILES: *[H]] | 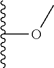 [SMILES: *OC] |
| 2-88 | 2a | 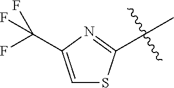 [SMILES: *c1nc(C(F)(F)F)cs1] | 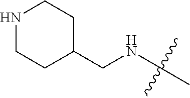 [SMILES: *NCC1CCNCC1] | 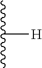 [SMILES: *[H]] | 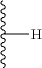 [SMILES: *[H]] |
| 2-89 | 2b | 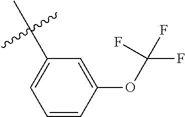 [SMILES: *c1cccc(OC(F)(F)F)c1] | 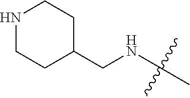 [SMILES: *NCC1CCNCC1] | 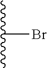 [SMILES: *Br] | 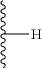 [SMILES: *[H]] |
| 2-90 | 2a | 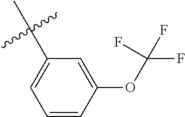 [SMILES: *c1cccc(OC(F)(F)F)c1] | 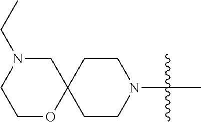 [SMILES: *N1CCC2(CC1)CN(CC)CCO2] | 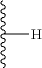 [SMILES: *[H]] | 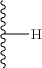 [SMILES: *[H]] |
| 2-91 | 2b | 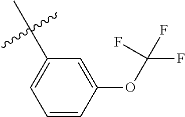 [SMILES: *c1cccc(OC(F)(F)F)c1] | 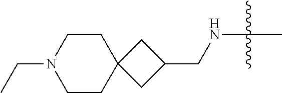 [SMILES: *NCC1CC2(CCN(CC)CC2)C1] | 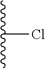 [SMILES: *Cl] | 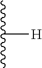 [SMILES: *[H]] |
| 2-92 | 2d or 2e | 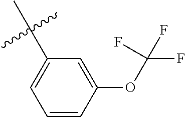 [SMILES: *c1cccc(OC(F)(F)F)c1] | 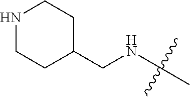 [SMILES: *NCC1CCNCC1] | 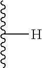 [SMILES: *[H]] | 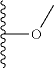 [SMILES: *OC] |
| 2-93 | 2d or 2e | 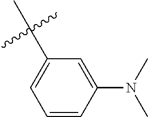 [SMILES: *c1cccc(N(C)C)c1] | 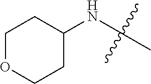 [SMILES: *NC1CCOCC1] | 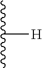 [SMILES: *[H]] | 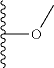 [SMILES: *OC] |
| 2-94 | 2d or 2e | 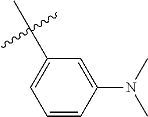 [SMILES: *c1cccc(N(C)C)c1] | 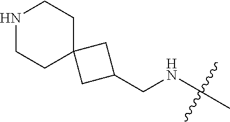 [SMILES: *NCC1CC2(CCNCC2)C1] | 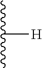 [SMILES: *[H]] | 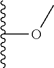 [SMILES: *OC] |
| 2-95 | 2a | 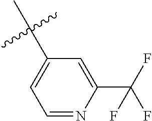 [SMILES: *c1ccnc(C(F)(F)F)c1] | 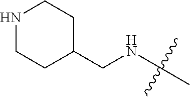 [SMILES: *NCC1CCNCC1] | 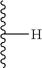 [SMILES: *[H]] | 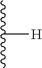 [SMILES: *[H]] |
| 2-96 | 2d or 2e | 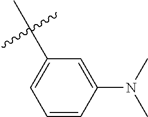 [SMILES: *c1cccc(N(C)C)c1] | 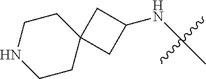 [SMILES: *NC1CC2(CCNCC2)C1] | 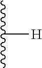 [SMILES: *[H]] | 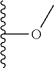 [SMILES: *OC] |

TABLE 2
Analytical data where Rt means retention time (in minutes), [M + H]⁺
means the protonated mass of the compound, method refers to the method
used for (LC)MS.

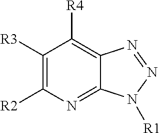

| No. | Rₜ | [M + 1]⁺ | Meth. | ¹H NMR (300 MHz; δ in ppm, J in Hz) |
|---|---|---|---|---|
| 2-01 | 4.06 | 324.1 | 1 | CDCl3 δ 8.34-8.15 (m, 2H), 8.04 (d, J = 9.3 |
| | | | | Hz, 1H), 7.21-6.94 (m, 2H), 6.68 (d, J = 9.3 |
| | | | | Hz, 1H), 3.89 (s, 3H), 3.76 (t, J = 5.8 Hz, |
| | | | | 4H), 1.86 (s, 4H), 1.74-1.43 (m, 4H) |
| 2-02 | 2.58 | 350.1 | 1 | CDCl3 δ 8.07 (m, 3H), 7.44-7.31 (m, 2H), |
| | | | | 7.12-6.95 (m, 4H), 6.52 (d, J = 9.0 Hz, 1H), |
| | | | | 5.31 (brs, 1H), 4.65 (d, J = 5.6 Hz, 2H), |
| | | | | 3.89 (s, 3H) |
| 2-03 | 3.17 | 366.1 | 1 | CDCl3 δ 8.09-7.92 (m, 3H), 7.40 (s, 1H), |
| | | | | 7.33-7.19 (m, 3H), 7.04 (d, J = 9.1 Hz, 2H), |
| | | | | 6.53 (d, J = 9.0 Hz, 1H), 5.38 (s, 1H), 4.64 |
| | | | | (d, J = 5.7 Hz, 2H), 3.88 (s, 3H) |
| 2-04 | 6.72 | 336.1 | 1 | Acetone δ 8.10-7.89 (m, 3H), 7.50-7.41 (m, |
| | | | | 3H), 7.17-6.93 (m, 3H), 6.78 (d, J = 9.1 Hz, |
| | | | | 1H), 4.67 (d, J = 5.6 Hz, 2H) |
| 2-05 | 5.58 | 310.1 | 2 | MeOD δ 8.00 (m, 3H), 6.97 (d, J = 8.9 Hz, |
| | | | | 2H), 6.87 (d, J = 9.4 Hz, 1H), 3.77 (t, J = |
| | | | | 5.9 Hz, 4H), 1.85 (brs, 4H), 1.57 (brm, 4H) |
| 2-06 | 7.2 | 422.1 | 2 | CDCl3 δ 8.97 (brs, 2H), 8.24-7.92 (m, 2H), |
| | | | | 7.37 (dd, J = 8.6, 5.4 Hz, 2H), 7.15-6.91 (m, |
| | | | | 3H), 6.00 (s, 1H), 4.73 (brs, 2H), 4.42 (q, |
| | | | | J = 7.1 Hz, 2H), 3.90 (3H, s), 1.45 (t, J = |
| | | | | 7.1 Hz, 3H) |
| 2-07 | 7.08 | 396.2 | 2 | Acetone δ 8.47 (s, 1H), 8.32-8.14 (m, 2H), |
| | | | | 7.32-6.99 (m, 2H), 4.39 (q, J = 7.1 Hz, 2H), |
| | | | | 3.90 (s, 3H), 3.78-3.37 (m, 4H), 1.92 (brs, |
| | | | | 4H), 1.61-1.50 (m, 4H), 1.40 (t, J = 7.1 Hz, |
| | | | | 3H) |
| 2-08 | 4.18 | 401.1 | 2 | MeOD δ 8.42 (s, 2H), 8.33 (m, 2H), 7.93 |
| | | | | (d, J = 9.1 Hz, 1H), 7.85-7.55 (m, 2H), |
| | | | | 7.44-7.15 (m, 2H), 6.63 (d, J = 9.1 Hz, 1H), |
| | | | | 3.76 (t, J = 7.0 Hz, 2H), 3.03 (t, J = 7.0 Hz, |
| | | | | 2H) |
| 2-09 | 6.73 | 338.1 | 2 | CDCl3 δ 8.52 (s, 1H), 8.33 (dd, J = 8.3, 1.0 |
| | | | | Hz, 1H), 8.01 (d, J = 9.0 Hz, 1H), 7.58 (t, |
| | | | | J = 8.3 Hz, 1H), 7.34-7.08 (m, 1H), 6.49 |
| | | | | (d, J = 9.0 Hz, 1H), 5.09 (s, 1H), 3.48 |
| | | | | (q, J = 7.4 Hz, 2H), 1.72 (sextet, J = 7.4 |
| | | | | Hz, 2H), 1.04 (t, J = 7.4 Hz, 3H) |
| 2-10 | 6.08 | 380.1 | 2 | CDCl3 δ 8.51 (s, 1H), 8.29 (d, J = 8.1 Hz, |
| | | | | 1H), 8.04 (d, J = 9.0 Hz, 1H), 7.59 (t, J = |
| | | | | 8.2 Hz, 1H), 7.26 (m, 1H), 6.50 (d, J = 9.0 |
| | | | | Hz, 1H), 4.95 (d, J = 6.2 Hz, 1H), 4.20 (brs, |
| | | | | 1H), 4.06 (d, J = 11.6 Hz, 2H), 3.59 (t, J = |
| | | | | 11.6 Hz, 2H), 3.59 (t, J = 11.6 Hz, 2H), |
| | | | | 2.15 (d, J = 12.3 Hz, 2H) |
| 2-11 | 6.31 | 316.2 | 2 | CDCl3 δ 8.41-8.38 (m, 2H), 8.05 (d, J = 9.0 |
| | | | | Hz, 1H), 7.64-7.59 (m, 2H), 7.49-7.28 (m, |
| | | | | 6H), 6.48 (d, J = 9.0 Hz, 1H), 5.07 (brs, |
| | | | | 1H), 3.82 (q, J = 7.1 Hz, 2H), 3.05 (t, J = |
| | | | | 7.1 Hz, 2H) |
| 2-12 | 6.87 | 312.2 | 2 | CDCl3 δ 8.37-8.32 (m, 2H), 8.06 (d, J = 9.3 |
| | | | | Hz, 1H), 7.27-7.22 (m, 2H), 6.70 (d, J = 9.3 |
| | | | | Hz, 1H), 3.77 (t, J = 5.8 Hz, 4H), 1.88 (m, |
| | | | | 4H), 1.63-1.46 (m, 4H) |
| 2-13 | 5.31 | 300.1 | 2 | CDCl3 δ 8.31-8.27 (m, 2H), 8.18 (d, J = 9.3 |
| | | | | Hz, 1H), 7.33-7.27 (m, 2H), 6.85 (d, J = 9.3 |
| | | | | Hz, 1H), 3.95-3.86 (m, 4H), 3.81-3.71 (m, |
| | | | | 4H) |
| 2-14 | 3.66 | 425.3 | 2 | Acetone δ 8.87 (s, 1H), 8.80 (t, J = 5.1 Hz, |
| | | | | 1H), 8.17 (d, J = 9.1 Hz, 2H), 7.20 (d, J = |
| | | | | 9.1 Hz, 2H), 4.42 (q, J = 7.1 Hz, 2H), 3.90 |
| | | | | (s, 3H), 3.69 (dd, J = 15.2, 9.0 Hz, 4H), |
| | | | | 3.21 (m, 2H), 3.01 (s, 3H), 2.27 (m, 1H), |
| | | | | 2.18 (m, 2H), 1.81 (m, 2H), 1.43 (t, J = 7.1 |
| | | | | Hz, 3H) |
| 2-15 | 6.3 | 408.2 | 2 | CDCl3 δ 8.95 (brm, 1H), 8.92 (s, 1H), 8.01- |
| | | | | 7.95 (m, 2H), 7.37 (dd, J = 8.6, 5.4 Hz, 2H), |
| | | | | 7.06-6.98 (m, 4H), 5.67 (s, 1H), 4.73 (d, J = |
| | | | | 5.5 Hz, 2H), 4.41 (q, J = 7.1 Hz, 2H), 1.43 |
| | | | | (t, J = 7.1 Hz, 3H) |
| 2-16 | 6.12 | 382.2 | 2 | CDCl3 δ 8.49 (s, 1H), 8.17-8.13 (m, 2H), |
| | | | | 7.04-7.01 (m, 2H), 5.80 (s, 1H), 4.41 (q, J = |
| | | | | 7.1 Hz, 2H), 3.58 (m, 4H), 1.90 (brs, 4H), |
| | | | | 1.55 (m, 4H), 1.42 (t, J = 7.1 Hz, 3H) |
| 2-17 | 6.35 | 394.2 | 2 | CDCl3 δ 8.37-8.32 (m, 2H), 7.99 (d, J = 9.0 |
| | | | | Hz, 1H), 7.30-7.24 (m, 2H), 7.05 (m, 1H), |
| | | | | 6.88-6.82 (m, 2H), 6.49 (d, J = 9.0 Hz, 1H), |
| | | | | 5.49 (s, 1H), 3.91 (s, 3H), 3.89 (s, 3H), 3.74 |
| | | | | (q, 6.8 Hz, 2H), 3.03 (t, J = 6.8 Hz, 2H) |
| 2-18 | 4.93 | 395.2 | 2 | Acetone δ 8.56-8.27 (m, 2H), 8.01 (d, J = |
| | | | | 9.1 Hz, 1H), 7.93-7.74 (m, 2H), 7.65 (dd, |
| | | | | J = 11.2, 4.6 Hz, 2H), 7.49 (dd, J = 7.9, 1.8 |
| | | | | Hz, 3H), 7.12 (s, 1H), 6.73 (d, J = 9.1 Hz, |
| | | | | 1H), 6.53 (s, 2H), 3.85-3.78 (m, 2H), 3.13 |
| | | | | (m, 2H) |
| 2-19 | 3.52 | 407.2 | 2 | CDCl3 δ 6 8.46 (s, 1H), 8.29 (ddd, J = 8.3, |
| | | | | 1.9, 0.8 Hz, 1H), 7.98 (d, J = 9.0 Hz, 1H), |
| | | | | 7.54 (t, J = 8.3 Hz, 1H), 7.23-7.19 (m, 1H), |
| | | | | 6.46 (d, J = 9.0 Hz, 1H), 5.17 (t, J = 5.6 |
| | | | | Hz, 1H), 3.40 (t, J = 6.1 Hz, 2H), 2.87 |
| | | | | (brm, 2H), 2.25 (s, 3H), 1.99-1.92 (m, 2H), |
| | | | | 1.80 (brm, 1H), 1.76-1.70 (m, 2H), 1.38 |
| | | | | (ddd, J = 14.8, 12.1, 3.7 Hz, 2H) |
| 2-20 | 3.01 | 350.1 | 2 | CDCl3) δ 8.47 (s, 1H), 8.29 (ddd, J = 8.2, |
| | | | | 1.9, 0.8 Hz, 1H), 7.99 (d, J = 9.0 Hz, 1H), |
| | | | | 7.55 (t, J = 8.3 Hz, 1H), 7.22-7.18 (m, 1H), |
| | | | | 6.48 (d, J = 9.0 Hz, 1H), 5.14 (s, 1H), 3.34 |
| | | | | (dd, J = 7.1, 5.2 Hz, 2H), 1.19-1.09 (m, |
| | | | | 1H), 0.75-0.45 (m, 2H), 0.29 (q, J = 4.7 Hz, |
| | | | | 2H) |
| 2-21 | 3.49 | 393.2 | 2 | CDC13 δ 8.48 (s, 1H), 8.29 (ddd, J = 8.3, |
| | | | | 1.9, 0.8 Hz, 1H), 7.97 (d, J = 9.0 Hz, 1H), |
| | | | | 7.54 (t, J = 8.3 Hz, 1H), 7.22-7.19 (m, 1H), |
| | | | | 6.47 (d, J = 9.0 Hz, 1H), 5.18 (t, J = 5.6 |
| | | | | Hz, 1H), 3.39 (t, J = 6.2 Hz, 2H), 3.10 (d, |
| | | | | J = 12.0 Hz, 2H), 2.59 (td, J = 12.1, 2.4 Hz, |
| | | | | 2H), 1.85-1.75 (m, 4H), 1.29-1.16 (m, 2H) |
| 2-22 | 3.54 | 478.3 | 2 | MeOD δ 8.46 (s, 1H), 8.31 (d, J = 8.3 Hz, |
| | | | | 1H), 7.93 (d, J = 9.1 Hz, 1H), 7.68 (t, J = |
| | | | | 8.4 Hz, 1H), 7.33 (d, J = 8.2 Hz, 1H), 6.70 |
| | | | | (d, J = 9.1 Hz, 1H), 4.08 (brs, 1H), 3.57- |
| | | | | 3.43 (m, 6H), 3.08-2.97 (m, 2H), 2.88 (s, |
| | | | | 3H), 2.84 (s, 3H), 2.13-2.01 (m, 2H), 1.75- |
| | | | | 1.67 (m, 2H) |
| 2-23 | 3.52 | 380.2 | 3 | DMSO δ 10.18 (s, 1H), 8.25 (s, 1H), 8.13 |
| | | | | (d, J = 8.9 Hz, 2H), 8.03 (d, J = 9.1 Hz, |
| | | | | 1H), 7.86-7.66 (m, 2H), 6.68 (d, J = 9.1 Hz, |
| | | | | 1H), 2.90 (d, J = 11.2 Hz, 2H), 2.26 (s, 3H), |
| | | | | 2.07 (brm, 7H), 1.73 (d, J = 11.4 Hz, 3H), |
| | | | | 1.31 (d, J = 10.7 Hz, 2H) |
| 2-24 | 4.59 | 270.2 | 2 | CDCl3 δ 8.06 (d, J = 7.8 Hz, 2H), 7.98 (d, |
| | | | | J = 9.0 Hz, 1H), 6.99 (d, J = 7.6 Hz, 2H), |
| | | | | 6.45 (d, J = 9.0 Hz, 1H), 4.96 (s, 1H), 3.41 |
| | | | | (q, J = 7.4 Hz, 2H), 1.68 (sexteto, J = 7.4 |
| | | | | Hz, 2H), 1.00 (t, J = 7.4 Hz, 3H) |
| 2-25 | 3.45 | 484.2 | 2 | CDCl3 δ 8.53-8.50 (m, 3H), 8.29 (ddd, J = |
| | | | | 8.3, 1.9, 0.8 Hz, 1H), 7.99 (d, J = 9.0 Hz, |
| | | | | 1H), 7.56 (t, J = 8.3 Hz, 1H), 7.26-7.21 (m, |
| | | | | 3H), 6.48 (d, J = 9.0 Hz, 1H), 5.18 (t, J = |
| | | | | 5.7 Hz, 1H), 3.48 (s, 2H), 3.43 (m, 2H), 2.87 |
| | | | | (d, J = 11.4 Hz, 2H), 2.04-1.96 (m, 2H), |
| | | | | 1.80-1.73 (m, 4H), 1.39 (dt, J = 12.4, 10.9 |
| | | | | Hz, 1H) |
| 2-26 | 3.75 | 475.2 | 2 | CDCl3 δ 8.48 (s, 1H), 8.29 (d, J = 8.3 Hz, |
| | | | | 1H), 7.98 (d, J = 9.0 Hz, 1H), 7.55 (t, J = |
| | | | | 8.3 Hz, 1H), 7.23 (m, 1H), 6.46 (d, J = 9.0 |
| | | | | Hz, 1H), 5.12 (s, 1H), 3.42 (t, J = 6.0 Hz, |
| | | | | 2H), 3.29 (s, 2H), 2.93 (d, J = 11.3 Hz, 2H), |
| | | | | 2.16 (ddd, J = 12.5, 7.9, 4.6 Hz, 1H), 2.11- |
| | | | | 2.00 (m, 2H), 1.78 (d, J = 12.2 Hz, 3H), |
| | | | | 1.47 (dd, J = 19.7, 7.9 Hz, 2H), 1.09-0.99 |
| | | | | (m, 2H), 0.86 (td, J = 7.4, 3.6 Hz, 2H) |
| 2-27 | 3.68 | 433.2 | 2 | CDCl3 δ 8.46 (s, 1H), 8.29 (ddd, J = 8.3, |
| | | | | 1.9, 0.8 Hz, 1H), 7.98 (d, J = 9.0 Hz, 1H), |
| | | | | 7.55 (t, J = 8.3 Hz, 1H), 7.23-7.19 (m, 1H), |
| | | | | 6.47 (d, J = 9.0 Hz, 1H), 5.87 (ddt, J = |
| | | | | 16.8, 10.1, 6.6 Hz, 1H), 5.19-5.12 (m, 3H), |
| | | | | 3.41 (t, J = 6.0 Hz, 2H), 2.99 (dd, J = 9.7, |
| | | | | 4.2 Hz, 3H), 1.99-1.91 (m, 3H), 1.82-1.72 |
| | | | | (m, 3H), 1.42 (td, J = 13.0, 6.9 Hz, 2H) |
| 2-28 | 3.56 | 439.2 | 2 | CDCl3 δ 8.47 (s, 1H), 8.29 (m, 1H), 7.98 (d, |
| | | | | J = 9.0 Hz, 1H), 7.55 (t, J = 8.3 Hz, 1H), |
| | | | | 7.22 (m, 1H), 6.46 (d, J = 9.0 Hz, 1H), 5.12 |
| | | | | (t, J = 5.6 Hz, 1H), 4.62 (t, J = 4.9 Hz, 1H), |
| | | | | 4.47 (t, J = 4.9 Hz, 1H), 3.41 (t, J = 6.0 Hz, |
| | | | | 2H), 2.99 (d, J = 11.6 Hz, 2H), 2.72 (t, J = |
| | | | | 4.9 Hz, 1H), 2.62 (t, J = 4.9 Hz, 1H), 2.10- |
| | | | | 2.02 (m, 2H), 1.81-1.71 (m, 3H), 1.41 (tt, |
| | | | | J = 13.9, 7.1 Hz, 2H) |
| 2-29 | 3.27 | 436.2 | 2 | CDCl3 δ 8.42 (s, 1H), 8.25-8.20 (m, 2H), |
| | | | | 8.15- 8.09 (m, 1H), 7.56 (t, J = 8.3 Hz, 1H), |
| | | | | 7.23-7.20 (m, 1H), 6.81 (d, J = 9.3 Hz, 1H), |
| | | | | 6.12 (s, 1H), 3.86-3.62 (m, 4H), 3.46 (dd, |
| | | | | J = 11.3, 5.6 Hz, 2H), 2.63-2.56 (m, 5H) |
| 2-30 | 3.67 | 451.2 | 2 | CDCl3 δ 8.46 (s, 1H), 8.29 (dd, J = 8.3, 1.1 |
| | | | | Hz, 1H), 7.98 (d, J = 9.0 Hz, 1H), 7.55 (t, |
| | | | | J = 8.3 Hz, 1H), 7.22 (dd, J = 7.0, 5.9 Hz, |
| | | | | 1H), 6.46 (d, J = 9.0 Hz, 1H), 5.16 (t, J = |
| | | | | 5.6 Hz, 1H), 3.51 (t, J = 5.6 Hz, 2H), 3.40 |
| | | | | (t, J = 5.9 Hz, 2H), 3.32 (s, 3H), 3.01 (d, |
| | | | | J = 11.5 Hz, 2H), 2.57 (t, J = 5.6 Hz, 2H), |
| | | | | 2.02 (t, J = 10.9 Hz, 2H), 1.77 (m, 3H), |
| | | | | 1.58-1.36 (m, 2H) |
| 2-31 | 3.77 | 447.3 | 2 | CDCl3 δ 8.46 (s, 1H), 8.29 (ddd, J = 8.3, |
| | | | | 1.9, 0.8 Hz, 1H), 7.98 (d, J = 9.0 Hz, 1H), |
| | | | | 7.55 (t, J = 8.3 Hz, 1H), 7.23-7.18 (m, 1H), |
| | | | | 6.47 (d, J = 9.0 Hz, 1H), 5.18 (t, J = 5.6 |
| | | | | Hz, 1H), 3.41 (t, J = 6.0 Hz, 2H), 3.12 (d, |
| | | | | J = 11.6 Hz, 2H), 2.26 (d, J = 6.6 Hz, 2H), |
| | | | | 2.02-1.94 (m, 3H), 1.77 (dd, J = 20.5, 8.3 |
| | | | | Hz, 2H), 1.52-1.40 (m, 2H), 0.91-0.83 (m, |
| | | | | 1H), 0.52-0.48 (m, 2H), 0.09 (q, J = 5.1 Hz, |
| | | | | 2H) |
| 2-32 | 0.37 | 325.2 | 3 | MeOD δ 7.91 (m, 3H), 6.94 (d, J = 8.9 Hz, |
| | | | | 2H), 6.64 (d, J = 9.1 Hz, 1H), 3.35 (brm, |
| | | | | 2H, partial overlap with MeOD-signal), 3.06 |
| | | | | (d, J = 12.6 Hz, 2H), 2.58 (t, J = 11.3 Hz, |
| | | | | 2H), 2.09-1.55 (m, 3H), 1.25 (d, J = 8.8 Hz, |
| | | | | 2H) |
| 2-33 | 7.12 | 479.3 | 2 | CDCl3) δ 8.45 (s, 1H), 8.26 (ddd, J = 8.3, |
| | | | | 1.9, 0.8 Hz, 1H), 8.00 (d, J = 9.0 Hz, 1H), |
| | | | | 7.55 (t, J = 8.3 Hz, 1H), 7.21 (m, 1H), 6.46 |
| | | | | (d, J = 9.0 Hz, 1H), 4.88 (d, J = 7.3 Hz, |
| | | | | 1H), 4.12-4.08 (m, 3H), 2.95 (t, J = 11.7 |
| | | | | Hz, 2H), 2.12 (d, J = 9.5 Hz, 2H), 1.46 (s, |
| | | | | 9H), 1.41 (brm, 1H) |
| 2-34 | 4.12 | 508.3 | 2 | CDCl3 δ 8.45 (s, 1H), 8.29 (dd, J = 8.2, 1.2 |
| | | | | Hz, 1H), 8.01 (t, J = 13.3 Hz, 1H), 7.55 (t, |
| | | | | J = 8.3 Hz, 1H), 7.21 (dd, J = 8.3, 1.0 Hz, |
| | | | | 1H), 6.51 (d, J = 9.0 Hz, 1H), 5.63 (s, 1H), |
| | | | | 3.56 (m, 2H), 3.46 (m, 4H), 2.67 (t, J = 5.9 |
| | | | | Hz, 2H), 2.48-2.44 (m, 4H), 1.44 (s, 9H) |
| 2-35 | 3.7 | 422.2 | 2 | CDCl3 δ 8.44 (s, 1H), 8.29 (d, J = 8.2 Hz, |
| | | | | 1H), 7.99 (d, J = 9.0 Hz, 1H), 7.56 (t, J = |
| | | | | 8.3 Hz, 1H), 7.23-7.19 (m, 1H), 6.52 (d, J = |
| | | | | 9.0 Hz, 1H), 6.30 (s, 1H), 5.60 (s, 1H), 3.61 |
| | | | | (dd, J = 11.0, 5.4 Hz, 2H), 3.38 (d, J = 4.0 |
| | | | | Hz, 2H), 3.22 (s, 2H), 2.75 (dd, J = 11.2, |
| | | | | 5.5 Hz, 4H) |
| 2-36 | 4.39 | 408.2 | 3 | DMSO δ 9.81 (s, 1H), 8.40 (d, J = 8.2 Hz, |
| | and | | | 1H), 8.27-8.15 (m, 2H), 7.84 (t, J = 8.3 Hz, |
| | 4.68 | | | 1H), 7.48 (d, J = 8.4 Hz, 1H), 6.77 (d, J = |
| | | | | 9.1 Hz, 1H), 3.85 (d, J = 5.0 Hz, 2H), 3.64- |
| | | | | 3.28 (brm, 11H, overlap with water signal) |
| 2-37 | 4.91 | 340.2 | 2 | MeOD δ 8.44 (s, 1H), 8.36-8.32 (m, 1H), |
| | | | | 7.95 (d, J = 9.1 Hz, 1H), 7.68 (t, J = 8.3 |
| | | | | Hz, 1H), 7.34-7.31 (m, 1H), 6.71 (d, J = 9.1 |
| | | | | Hz, 1H), 3.80 (t, J = 5.6 Hz, 2H), 3.62 (t, |
| | | | | J = 5.6 Hz, 2H) |
| 2-38 | 5.09 | 354.2 | 2 | MeOD δ 8.46 (s, 1H), 8.35 (dd, J = 8.3, 1.1 |
| | | | | Hz, 1H), 7.91 (d, J = 9.1 Hz, 1H), 7.66 (t, |
| | | | | J = 8.3 Hz, 1H), 7.31 (d, J = 8.2 Hz, 1H), |
| | | | | 6.64 (d, J = 9.1 Hz, 1H), 3.69 (t, J = 6.4 |
| | | | | Hz, 2H), 3.56 (t, J = 6.9 Hz, 2H), 1.91 (p, |
| | | | | J = 6.6 Hz, 2H) |
| 2-39 | 3.02 | 367.2 | 3 | MeOD δ 8.42 (s, 1H), 8.35 (d, J = 8.2 Hz, |
| | | | | 1H), 7.94 (d, J = 9.1 Hz, 1H), 7.67 (t, J = |
| | | | | 8.3 Hz, 1H), 7.34 (d, J = 8.0 Hz, 1H), 6.66 |
| | | | | (d, J = 9.1 Hz, 1H), 3.64 (t, J = 6.8 Hz, |
| | | | | 2H), 2.67 (t, J = 6.8 Hz, 2H), 2.34 (s, 6H) |
| 2-40 | 4.29 | 353.1 | 2 | MeOD δ 8.43 (ddd, J = 8.3, 1.9, 0.8 Hz, |
| | | | | 1H), 8.28 (s, 1H), 8.04 (d, J = 9.1 Hz, 1H), |
| | | | | 7.69 (t, J = 8.3 Hz, 1H), 7.39-7.27 (m, 1H), |
| | | | | 6.80 (d, J = 9.1 Hz, 1H), 4.11 (s, 2H) |
| 2-41 | 4.79 | 381.2 | 2 | MeOD δ 8.40 (d, J = 5.2 Hz, 1H), 8.37 (brs, |
| | | | | 1H), 7.96 (d, J = 9.1 Hz, 1H), 7.70 (t, J = |
| | | | | 8.2 Hz, 1H), 7.34 (d, J = 8.2 Hz, 1H), 6.67 |
| | | | | (d, J = 9.1 Hz, 1H), 3.62 (t, J = 5.9 Hz, |
| | | | | 2H), 3.48 (t, J = 6.0 Hz, 2H), 1.91 (s, 3H) |
| 2-42 | 4.6 | 367.2 | 2 | MeOD δ 8.44 (s, 1H), 8.38 (ddd, J = 8.3, |
| | | | | 1.8, 0.8 Hz, 1H), 7.94 (d, J = 9.1 Hz, 1H), |
| | | | | 7.68 (t, J = 8.3 Hz, 1H), 7.39-7.28 (m, 1H), |
| | | | | 6.67 (d, J = 9.1 Hz, 1H), 3.77 (t, J = 6.6 |
| | | | | Hz, 2H), 2.63 (t, J = 6.6 Hz, 2H) |
| 2-43 | 3.36 | 405.2 | 2 | CDCl3 δ 8.45 (s, 1H), 8.29 (d, J = 8.2 Hz, |
| | | | | 1H), 8.11 (d, J = 9.3 Hz, 1H), 7.60 (t, J = |
| | | | | 8.3 Hz, 1H), 7.25 (d, J = 14.2 Hz, 1H), 6.86 |
| | | | | (d, J = 9.3 Hz, 1H), 3.73 (m, 8H), 2.00 (m, |
| | | | | 4H) |
| 2-44 | 3.29 | 393.2 | 2 | MeOD δ 8.42 (s, 1H), 8.29 (dd, J = 7.9, 1.5 |
| | | | | Hz, 1H), 8.08 (t, J = 9.1 Hz, 1H), 7.69 (t, |
| | | | | J = 8.3 Hz, 1H), 7.33 (d, J = 8.2 Hz, 1H), |
| | | | | 7.07 (t, J = 9.0 Hz, 1H), 4.58 (d, J = 13.4 |
| | | | | Hz, 2H), 3.17-3.08 (m, 2H), 2.90-2.79 (m, |
| | | | | 1H), 2.47 (s, 3H), 2.08 (d, J = 11.8 Hz, 2H), |
| | | | | 1.49-1.36 (m, 2H) |
| 2-45 | 3.55 | 433.3 | 2 | CDCl3 δ 8.44 (s, 1H), 8.27 (ddd, J = 8.3, |
| | | | | 1.9, 0.8 Hz, 1H), 8.06 (dd, J = 9.3, 4.7 Hz, |
| | | | | 1H), 7.56 (t, J = 8.3 Hz, 1H), 7.28-7.13 (m, |
| | | | | 1H), 6.88-6.75 (m, 1H), 3.75-3.69 (m, 4H), |
| | | | | 2.99-2.94 (m, 4H), 1.61 (brm, 8H) |
| 2-46 | 3.25 | 391.2 | 2 | MeOD δ 8.31 (s, 1H), 8.20 (m, 1H), 7.85 (t, |
| | | | | J = 7.1 Hz, 1H), 7.52 (t, J = 8.3 Hz, 1H), |
| | | | | 7.17 (d, J = 8.3 Hz, 1H), 6.52 (d, J = 9.2 |
| | | | | Hz, 1H), 3.65 (dd, J = 11.3, 7.7 Hz, 2H), |
| | | | | 3.37 (dd, J = 11.5, 3.1 Hz, 2H), 3.09 (dd, |
| | | | | J = 11.3, 7.1 Hz, 2H), 3.01-2.89 (m, 2H), |
| | | | | 2.77 (dd, J = 11.4, 3.4 Hz, 2H) |
| 2-47 | 5.3 | 394.2 | 2 | Acetone δ 8.59 (s, 1H), 8.42 (ddd, J = 8.3, |
| | | | | 1.9, 0.8 Hz, 1H), 7.99 (d, J = 9.1 Hz, 1H), |
| | | | | 7.77 (t, J = 8.3 Hz, 1H), 7.42-7.39 (m, 1H), |
| | | | | 6.87 (d, J = 6.9 Hz, 1H), 6.71 (d, J = 9.1 |
| | | | | Hz, 1H), 4.00 (dd, J = 7.0, 3.6 Hz, 1H), 3.62 |
| | | | | (td, J = 9.8, 4.9 Hz, 1H), 2.21 (d, J = 10.8 |
| | | | | Hz, 2H), 2.00 (d, J = 10.7 Hz, 2H), 1.54- |
| | | | | 1.34 (m, 4H) |
| 2-48 | 4.96 | 408.2 | 2 | DMSO δ 8.40 (s, 1H), 8.36 (d, J = 8.1 Hz, |
| | | | | 1H), 8.09 (d, J = 9.1 Hz, 1H), 7.91 (s, 1H), |
| | | | | 7.78 (t, J = 8.3 Hz, 1H), 7.46 (d, J = 8.1 |
| | | | | Hz, 1H), 6.71 (d, J = 9.1 Hz, 1H), 6.30 (brs, |
| | | | | 1H), 3.52 (dd, J = 12.0, 6.0 Hz, 2H), 3.40 |
| | | | | (dd, J = 9.0, 6.7 Hz, 2H), 3.31 (q, J = 7.8 |
| | | | | Hz, 2H; parcial overlap with water signal), |
| | | | | 3.18 (t, J = 7.8 Hz, 2H) |
| 2-49 | 3.31 | 405.2 | 2 | MeOD δ 8.46 (s, 1H), 8.34 (d, J = 8.2 Hz, |
| | | | | 1H), 7.95 (d, J = 9.2 Hz, 1H), 7.69 (t, J = |
| | | | | 8.3 Hz, 1H), 7.35 (d, J = 8.2 Hz, 1H), 6.67 |
| | | | | (d, J = 9.1 Hz, 1H), 3.56 (t, J = 6.7 Hz, |
| | | | | 2H), 3.30 (m, 2H, overlap with MeOD- |
| | | | | signal), 2.86-2.53 (m, 2H), 2.38 (s, 4H), |
| | | | | 1.97-1.88 (m, 2H) |
| 2-50 | 3.1 | 367.2 | 2 | MeOD δ 8.40 (s, 1H), 8.32 (d, J = 8.2 Hz, |
| | | | | 1H), 8.04 (d, J = 9.1 Hz, 1H), 7.64 (t, J = |
| | | | | 8.3 Hz, 1H), 7.31 (d, J = 8.3 Hz, 1H), 6.52 |
| | | | | (d, J = 9.1 Hz, 1H), 3.99 (s, 3H), 3.23 (brm, |
| | | | | 2H), 3.12-3.08 (m, 2H), 2.14-2.07 (brm, |
| | | | | 2H) |
| 2-51 | 5.41 | 433.2 | 2 | MeOD δ 8.43 (s, 1H), 8.31 (ddd, J = 8.3, |
| | | | | 1.9, 0.8 Hz, 1H), 8.11 (d, J = 9.4 Hz, 1H), |
| | | | | 7.69 (t, J = 8.3 Hz, 1H), 7.39-7.30 (m, 1H), |
| | | | | 7.10 (d, J = 9.5 Hz, 1H), 4.46 (dt, J = 13.9, |
| | | | | 3.9 Hz, 2H), 3.44-3.34 (m, 4H), 2.23 (t, J = |
| | | | | 6.9 Hz, 2H), 2.04-1.75 (m, 2H), 1.61 (d, J = |
| | | | | 13.5 Hz, 2H) |
| 2-52 | 5.21 | 433.2 | 2 | MeOD δ 8.43 (s, 1H), 8.31 (dd, J = 8.3, 1.1 |
| | | | | Hz, 1H), 8.11 (d, J = 9.4 Hz, 1H), 7.70 (t, |
| | | | | J = 8.3 Hz, 1H), 7.34 (d, J = 8.3 Hz, 1H), |
| | | | | 7.11 (d, J = 9.5 Hz, 1H), 3.98-3.90 (m, 2H), |
| | | | | 3.81-3.72 (m, 2H), 3.30 (2H, m, overlap |
| | | | | MeOD-signal), 2.35 (s, 2H), 1.79 (t, J = 5.6 |
| | | | | Hz, 4H) |
| 2-53 | 3.15 | 379.2 | 2 | MeOD δ 8.54 (s, 1H), 8.31 (d, J = 9.7 Hz, |
| | | | | 1H), 7.93 (d, J = 9.2 Hz, 1H), 7.68 (t, J = |
| | | | | 8.3 Hz, 1H), 7.34 (d, J = 8.2 Hz, 1H), 6.66 |
| | | | | (d, J = 9.1 Hz, 1H), 4.17-3.98 (m, 1H), 3.12 |
| | | | | (dt, J = 12.7, 3.4 Hz, 2H), 2.75 (td, J = |
| | | | | 12.6, 2.5 Hz, 2H), 2.12 (d, J = 10.0 Hz, |
| | | | | 2H), 1.57-1.48 (m, 2H) |
| 2-54 | 3.5 | 427.2 | 2 | CDCl3 δ 8.46 (s, 1H), 8.27 (d, J = 7.9 Hz, |
| | | | | 1H), 8.12 (s, 1H), 7.57 (t, J = 8.2 Hz, 1H), |
| | | | | 5.78 (brs, 1H), 3.49 (t, J = 5.9 Hz, 2H), 3.11 |
| | | | | (d, J = 11.7 Hz, 2H), 2.60 (t, J = 11.8 Hz, |
| | | | | 2H), 1.79 (m, 2H), 1.26-1.19 (m, 3H) |
| 2-55 | 3.55 | 441.2 | 2 | CDCl3 δ 8.42 (s, 1H), 8.26 (d, J = 8.3 Hz, |
| | | | | 1H), 8.12 (d, J = 2.8 Hz, 1H), 7.57 (t, J = |
| | | | | 8.3 Hz, 1H), 7.23 (d, J = 2.8 Hz, 1H), 5.80 |
| | | | | (brs, 1H), 3.51 (t, J = 5.8 Hz, 2H), 2.94 (d, |
| | | | | J = 10.1 Hz, 2H), 2.31 (s, 3H), 2.00 (t, J = |
| | | | | 11.7 Hz, 2H), 1.81 (d, J = 11.0 Hz, 2H), |
| | | | | 1.50 (m, 3H) |
| 2-56 | 5.79 | 457.3 | 2 | MeOD δ 8.49 (s, 1H), 8.33 (dd, J = 8.3, 1.1 |
| | | | | Hz, 1H), 7.97 (d, J = 9.1 Hz, 1H), 7.70 (t, |
| | | | | J = 8.3 Hz, 1H), 7.35 (d, J = 8.3 Hz, 1H), |
| | | | | 6.69 (d, J = 9.1 Hz, 1H), 4.11 (brm, 1H), |
| | | | | 3.80-3.71 (m, 2H), 3.08-2.95 (m, 2H), 2.87 |
| | | | | (s, 3H), 2.24 (brm, 2H), 1.69 (td, J = 14.7, |
| | | | | 3.9 Hz, 2H) |
| 2-57 | 5.73 | 408.2 | 2 | CDCl3 δ 8.47 (s, 1H), 8.27 (dd, J = 8.2, 1.1 |
| | | | | Hz, 1H), 7.99 (d, J = 9.0 Hz, 1H), 7.55 (t, |
| | | | | J = 8.3 Hz, 1H), 7.22 (d, J = 10.6 Hz, 1H), |
| | | | | 6.48 (dd, J = 9.0, 4.1 Hz, 1H), 3.58 (ddd, |
| | | | | J = 15.0, 10.6, 4.3 Hz, 1H), 3.36 (d, J = 6.7 |
| | | | | Hz, 2H), 1.96 (m, 4H), 1.63 (m, 1H), 1.34- |
| | | | | 1.04 (m, 4H) |
| 2-58 | 3.64 | 435.3 | 2 | CDCl3 δ 8.36 (s, 1H), 8.23 (d, J = 7.8 Hz, |
| | | | | 1H), 8.11 (d, J = 9.2 Hz, 1H), 7.67-7.48 (m, |
| | | | | 1H), 7.22 (d, J = 9.6 Hz, 1H), 6.77 (d, J = |
| | | | | 9.1 Hz, 1H), 3.80 (m, 4H), 3.62 (s, 1H), 3.02 |
| | | | | (brm, 5H), 1.99 (m, 2H), 1.76 (s, 2H) |
| 2-59 | 3.79 | 433.3 | 2 | CDCl3 δ 9.49 (brs, 1H), 8.18 (m, 2H), 7.70 |
| | | | | (s, 1H), 7.24 (s, 1H), 6.81 (s, 1H), 3.69 (m, |
| | | | | 4H), 3.17 (brm, 3H), 2.52-1.00 (m, 10H) |
| 2-60 | 3.63 | 419.3 | 2 | CDCl3 δ 8.58 (s, 1H), 8.30 (d, J = 7.8 Hz, |
| | | | | 1H), 8.02 (d, J = 9.1 Hz, 1H), 7.54 (t, J = |
| | | | | 8.1 Hz, 1H), 7.37-6.95 (m, 1H), 6.50 (d, J = |
| | | | | 9.1 Hz, 1H), 3.54 (brm, 4H), 3.14-2.62 (m, |
| | | | | 4H), 2.26 (s, 2H), 1.95 (t, J = 6.6 Hz, 2H), |
| | | | | 1.63 (s, 2H) |
| 2-61 | 3.59 | 435.4 | 2 | CDCl3 δ 8.42 (brm, 1H), 8.25 (brm, 1H), |
| | | | | 8.05 (brm, 1H), 7.55 (brm, 1H), 7.22 (d, J = |
| | | | | 8.8 Hz, 1H), 6.83 (s, 1H), 4.15 (m, 2H), 3.75 |
| | | | | (m, 3H), 3.44 (s, 2H), 2.88 (brm, 3H), 2.08 |
| | | | | (m, 2H), 1.58 (brm, 2H). |
| 2-62 | 3.64 | 419.3 | 2 | CDCl3 δ 8.43 (s, 1H), 8.26 (d, J = 8.1 Hz, |
| | | | | 1H), 8.06 (d, J = 9.3 Hz, 1H), 7.55 (t, J = |
| | | | | 8.2 Hz, 1H), 7.36-7.06 (m, 1H), 6.82 (d, J = |
| | | | | 9.3 Hz, 1H), 3.74 (dt, J = 13.0, 10.6 Hz, |
| | | | | 4H), 3.12 (t, J = 6.8 Hz, 2H), 2.88 (s, 4H), |
| | | | | 1.73 (m, 4H) |
| 2-63 | 3.33 | 377.2 | 2 | MeOD δ 8.87 (s, 1H), 8.54 (d, J = 8.0 Hz, |
| | | | | 1H), 7.93 (d, J = 9.1 Hz, 1H), 7.87-7.63 (m, |
| | | | | 2H), 6.67 (d, J = 9.2 Hz, 1H), 3.41-3.34 (m, |
| | | | | 2H), 3.09 (s, 2H), 2.63 (td, J = 12.4, 2.4 Hz, |
| | | | | 2H), 2.13-1.68 (m, 3H), 1.27 (qd, J = 12.8, |
| | | | | 3.9 Hz, 3H) |
| 2-64 | 3.43 | 405.2 | 2 | DMSO δ 9.29 (s, 1H), 8.45 (s, 1H), 8.36 (d, |
| | | | | J = 8.2 Hz, 1H), 8.28 (d, J = 9.2 Hz, 1H), |
| | | | | 7.78 (t, J = 8.3 Hz, 1H), 7.46 (d, J = 6.8 |
| | | | | Hz, 1H), 6.75 (d, J = 9.3 Hz, 1H), 3.68 |
| | | | | (brm, 3H), 3.57 (d, J = 11.5 Hz, 1H), 3.19 |
| | | | | (t, J = 5.6 Hz, 2H), 2.21-2.04 (m, 2H), 2.00 |
| | | | | (t, J = 7.4 Hz, 2H) |
| 2-65 | 3.68 | 433.2 | 2 | DMSO δ 8.53 (s, 1H), 8.27 (d, J = 9.3 Hz, |
| | | | | 1H), 8.07 (d, J = 9.1 Hz, 1H), 7.91 (s, 1H), |
| | | | | 7.77 (t, J = 8.3 Hz, 1H), 7.45 (d, J = 8.6 |
| | | | | Hz, 1H), 6.71 (d, J = 9.1 Hz, 1H), 2.91 |
| | | | | (brm, 5H), 2.63-2.57 (m, 2H), 1.98-1.92 |
| | | | | (m, 2H), 1.71 (brm, 2H), 1.64-1.53 (m, 5H) |
| 2-66 | 3.51 | 419.4 | 2 | DMSO δ 8.62 (s, 1H), 8.56 (s, 1H), 8.28 (d, |
| | | | | J = 9.4 Hz, 1H), 8.20-8.06 (m, 2H), 7.77 (t, |
| | | | | J = 8.3 Hz, 1H), 7.47 (d, J = 7.5 Hz, 1H), |
| | | | | 6.70 (d, J = 9.1 Hz, 1H), 4.42-4.30 (m, |
| | | | | 1H), 3.00 (brm, 4H), 2.43-2.36 (m, 2H), |
| | | | | 1.91-1.64 (m, 6H) |
| 2-67 | 3.72 | 433.4 | 2 | DMSO δ 8.52 (s, 1H), 8.27 (d, J = 7.9 Hz, |
| | | | | 1H), 8.07 (d, J = 9.1 Hz, 1H), 7.91 (s, 1H), |
| | | | | 7.76 (t, J = 8.0 Hz, 1H), 7.45 (d, J = 7.2 |
| | | | | Hz, 1H), 6.71 (d, J = 9.1 Hz, 1H), 3.40 (m, |
| | | | | 4H), 2.92 (m, 4H), 1.96 (m, 2H), 1.63 (m, |
| | | | | 4H) |
| 2-68 | 5.70 | 394.2 | 2 | MeOD δ 8.56 (s, 1H), 8.34 (d, J = 8.3 Hz, |
| | | | | 1H), 7.92 (d, J = 9.2 Hz, 1H), 7.68 (t, J = |
| | | | | 8.3 Hz, 1H), 7.34 (d, J = 7.4 Hz, 1H), 6.70 |
| | | | | (d, J = 9.2 Hz, 1H), 4.02 (m, 1H), 3.91 (m, |
| | | | | 1H), 1.98-1.66 (m, 8H) |
| 2-69 | 3.20 | 365.3 | 2 | MeOD δ 8.33 (s, 1H), 8.31 (d, J = 10.8 Hz, |
| | | | | 1H), 8.27 (d, J = 9.4 Hz, 1H), 7.73 (t, J = |
| | | | | 8.2 Hz, 1H), 7.39 (d, J = 8.1 Hz, 1H), 7.21 |
| | | | | (d, J = 9.3 Hz, 1H), 4.09 (m, 4H), 3.41 (m, |
| | | | | 4H). |
| 2-70 | 5.56 | 380.1 | 2 | MeOD δ 8.44 (s, 1H), 8.30 (d, J = 8.2 Hz, |
| | | | | 1H), 8.08 (d, J = 9.4 Hz, 1H), 7.69 (t, J = |
| | | | | 8.3 Hz, 1H), 7.33 (d, J = 7.9 Hz, 1H), 7.08 |
| | | | | (d, J = 9.4 Hz, 1H), 4.28 (m, 2H), 3.93 (m, |
| | | | | 1H), 3.41 (ddd, J = 20.4, 11.7, 6.8 Hz, 2H), |
| | | | | 1.98 (m, 2H), 1.57 (m, 2H). |
| 2-71 | 0.37 | 325.3 | 3 | DMSO δ 8.81 (s, 1H), 8.59 (m, 2H), 8.33 |
| | | | | (m, 1H), 8.08 (d, J = 8.9 Hz, 1H), 7.97 (s, |
| | | | | 1H), 7.18 (d, J = 9.1 Hz, 1H), 6.72 (d, J = |
| | | | | 9.0 Hz, 1H), 3.30 (m, 4H), 2.85 (m, 2H), |
| | | | | 1.88 (m, 3H), 1.39 (m, 2H). |
| 2-72 | 5.80 | 443.1 | 2 | MeOD δ 8.36 (s, 1H), 8.31 (d, J = 8.3 Hz, |
| | | | | 1H), 8.18 (d, J = 9.4 Hz, 1H), 7.71 (t, J = |
| | | | | 8.3 Hz, 1H), 7.36 (d, J = 8.1 Hz, 1H), 7.14 |
| | | | | (d, J = 9.4 Hz, 1H), 3.94 (m, 4H), 3.37 |
| | | | | (m, 4H), 2.88 (s, 3H). |
| 2-73 | 3.53 | 435.4 | 2 | CDCl₃ δ 8.47 (s, 1H), 8.29 (d, J = 8.0 Hz, |
| | | | | 1H), 7.98 (d, J = 9.0 Hz, 1H), 7.55 (t, J = |
| | | | | 8.2 Hz, 1H), 7.22 (m, 1H), 6.50 (d, J = 9.0 |
| | | | | Hz, 1H), 5.32 (s, 1H), 3.42 (t, J = 5.7 Hz, |
| | | | | 2H), 2.99 (d, J = 11.3 Hz, 2H), 2.83 (m, |
| | | | | 1H), 2.22 (m, 2H), 1.84 (m, 3H), 1.52 (m, |
| | | | | 2H), 1.08 (d, J = 6.6 Hz, 6H). |
| 2-74 | 3.71 | 449.4 | 2 | DMSO δ 8.48 (s, 1H), 8.32 (d, J = 8.2 Hz, |
| | | | | 1H), 8.11 (m, 2H), 7.80 (t, J = 8.3 Hz, 1H), |
| | | | | 7.47 (d, J = 8.4 Hz, 1H), 6.76 (d, J = 9.1 |
| | | | | Hz, 1H), 3.43 (m, 4H), 3.33 (m, 2H), 2.84 |
| | | | | (m, 3H), 2.08 (m, 1H), 1.88 (m, 3H), 1.67 |
| | | | | (m, 2H), 0.96 (d, J = 5.4 Hz, 6H). |
| 2-75 | 3.64 | 491.3 | 2 | DMSO δ 8.46 (s, 1H), 8.32 (d, J = 8.1 Hz, |
| | | | | 1H), 8.11 (m, 2H), 7.79 (m, 1H), 7.47 (d, |
| | | | | J = 8.2 Hz, 1H), 6.77 (d, J = 7.3 Hz, 1H), |
| | | | | 3.83 (m, 2H), 3.51 (m, 2H), 3.29 (m, 2H), |
| | | | | 3.22 (m, 2H), 2.90 (m, 4H), 2.02 (m, 1H), |
| | | | | 1.88 (m, 3H), 1.69 (m, 4H), 1.21 (m, 2H). |
| 2-76 | 3.45 | 421.0 | 2 | CDCl₃ δ 8.47 (s, 1H), 8.28 (d, J = 8.2 Hz, |
| | | | | 1H), 7.97 (d, J = 9.0 Hz, 1H), 7.55 (t, J = |
| | | | | 8.3 Hz, 1H), 7.20 (m, 1H), 6.52 (d, J = 9.0 |
| | | | | Hz, 1H), 5.44 (s, 1H), 3.43 (m, 2H), 3.11 |
| | | | | (d, J = 11.4 Hz, 2H), 2.53 (dd, J = 14.4, |
| | | | | 7.1 Hz, 2H), 2.07 (t, J = 11.3 Hz, 2H), 1.85 |
| | | | | (m, 3H), 1.60 (m, 2H), 1.15 (t, J = 7.2 Hz, |
| | | | | 3H). |
| 2-77 | 3.91 | 467.2 | 2 | DMSO δ 8.67 (s, 1H), 8.29 (m, 3H), 7.81 (t, |
| | | | | J = 8.2 Hz, 1H), 7.51 (d, J = 8.1 Hz, 1H), |
| | | | | 3.89 (m, 1H), 3.72 (m, 2H), 3.63 (m, 1H), |
| | | | | 3.22 (m, 2H), 2.27 (m, 2H), 1.90 (m, 7H). |
| 2-78 | 3.99 | 467.2 | 2 | DMSO δ 8.52 (s, 1H), 8.44 (s, 1H), 8.26 (d, |
| | | | | J = 8.3 Hz, 1H), 7.79 (t, J = 8.3 Hz, 1H), |
| | | | | 7.72 (t, J = 5.6 Hz, 1H), 7.49 (d, J = 8.7 |
| | | | | Hz, 1H), 3.55 (t, J = 6.4 Hz, 2H), 2.94 (s, |
| | | | | 2H), 2.87 (s, 2H), 2.71 (m, 1H), 1.91 (m, |
| | | | | 2H), 1.69 (s, 2H), 1.61 (m, 4H). |
| 2-79 | 3.77 | 453.0 | 2 | DMSO δ 8.54 (s, 1H), 8.47 (s, 1H), 8.26 (d, |
| | | | | J = 8.2 Hz, 1H), 7.80 (t, J = 8.3 Hz, 1H), |
| | | | | 7.69 (d, J = 6.0 Hz, 1H), 7.51 (d, J = 7.9 |
| | | | | Hz, 1H), 4.50 (m, 1H), 3.04 (m, 2H), 2.95 |
| | | | | (m, 2H), 2.36 (m, 2H), 2.03 (m, 2H), 1.81 |
| | | | | (m, 2H), 1.75 (m, 2H). |
| 2-80 | 5.59 | 457.0 | 2 | DMSO δ 8.40 (s, 1H), 8.26 (m, 2H), 7.79 (t, |
| | | | | J = 8.3 Hz, 1H), 7.47 (d, J = 7.4 Hz, 1H), |
| | | | | 7.19 (d, J = 9.4 Hz, 1H), 7.14 (d, J = 7.3 |
| | | | | Hz, 1H), 4.38 (m, 2H), 3.52 (m, 1H), 3.25 |
| | | | | (m, 2H), 2.96 (s, 3H), 1.95 (m, 2H), 1.47 |
| | | | | (m, 2H). |
| 2-81 | 3.65 | 417.1 | 2 | DMSO δ 8.91 (m, 1H), 8.81 (s, 1H), 8.57 (d, |
| | | | | J = 7.6 Hz, 1H), 8.30 (d, J = 9.3 Hz, 1H), |
| | | | | 7.87 (m, 2H), 7.03 (d, J = 9.3 Hz, 1H), 4.04 |
| | | | | (m, 1H), 3.76 (m, 3H), 3.22 (s, 2H), 1.95 (m, |
| | | | | 9H). |
| 2-82 | 3.61 | 417.1 | 2 | DMSO δ 8.88 (s, 1H), 8.54 (d, J = 8.0 Hz, |
| | | | | 1H), 8.08 (d, J = 9.1 Hz, 1H), 7.95 (s, 1H), |
| | | | | 7.86 (m, 2H), 6.72 (d, J = 9.1 Hz, 1H), 3.45 |
| | | | | (t, J = 6.2 Hz, 2H), 2.95 (s, 2H), 2.87 (s, |
| | | | | 2H), 2.63 (m, 1H), 1.95 (m, 2H), 1.73 (m, |
| | | | | 2H), 1.64 (m, 2H), 1.57 (m, 2H). |
| 2-83 | 3.48 | 411.0 | 2 | DMSO δ 8.73 (s, 1H), 8.53 (m, 2H), 7.89 |
| | | | | (m, 3H), 3.40 (t, J = 6.1 Hz, 2H), 3.25 (m, |
| | | | | 2H), 2.79 (m, 2H), 2.12 (m, 1H), 1.81 (m, |
| | | | | 2H), 1.42 (m, 2H). |
| 2-84 | 3.52 | 403.0 | 2 | DMSO δ 8.98 (s, 1H), 8.52 (d, J = 8.0 Hz, |
| | | | | 1H), 8.19 (m, 1H), 8.12 (d, J = 9.1 Hz, 1H), |
| | | | | 7.86 (m, 2H), 6.71 (d, J = 9.1 Hz, 1H), 4.34 |
| | | | | (m, 1H), 3.05 (m, 2H), 2.95 (m, 2H), 2.41 |
| | | | | (m, 2H), 1.80 (m, 4H), 1.72 (m, 2H). |
| 2-85 | 6.18 | 428.0 | 2 | CDCl₃ δ 8.42 (s, 1H), 8.27 (d, J = 8.2 Hz, |
| | | | | 1H), 8.12 (s, 1H), 7.57 (t, J = 8.3 Hz, 1H), |
| | | | | 7.26 (m, 1H), 5.48 (d, J = 7.2 Hz, 1H), 4.02 |
| | | | | (m, 1H), 3.72 (m, 1H), 2.25 (m, 2H), 2.08 |
| | | | | (m, 2H), 1.41 (m, 4H). |
| 2-86 | 2.73 | 352.3 | 2 | DMSO δ 7.99 (d, J = 9.1 Hz, 1H), 7.80 (m, |
| | | | | 1H), 7.69 (s, 1H), 7.41 (d, J = 8.0 Hz, 1H), |
| | | | | 7.32 (t, J = 8.1 Hz, 1H), 6.71 (dd, J = 8.2, |
| | | | | 2.1 Hz, 1H), 6.65 (d, J = 9.1 Hz, 1H), 3.16 |
| | | | | (m, 4H), 2.95 (s, 6H), 2.76 (t, J = 11.5 Hz, |
| | | | | 2H), 1.84 (m, 1H), 1.79 (m, 2H), 1.31 (m, |
| | | | | 2H). |
| 2-87 | 2.83 | 382.3 | 2 | DMSO δ 8.01 (s, 1H), 7.70 (m, 2H), 7.50 (t, |
| | | | | J = 8.1 Hz, 1H), 7.07 (m, 1H), 6.19 (s, 1H), |
| | | | | 3.31 (m, 2H), 3.24 (m, 2H), 3.06 (s, 6H), |
| | | | | 2.82 (m, 2H), 1.85 (m, 3H), 1.41 (m, 2H). |
| 2-88 | 2.85 | 384.1 | 2 | MeOD δ 8.22 (s, 1H), 8.01 (d, J = 9.2 Hz, |
| | | | | 1H), 6.72 (d, J = 9.2 Hz, 1H), 3.52 (m, 2H), |
| | | | | 3.43 (m, 2H), 3.03 (m, 2H), 2.22 (m, 1H), |
| | | | | 2.10 (m, 2H), 1.54 (rn, 2H). |
| 2-89 | 3.73 | 473.1 | 2 | DMSO δ 8.64 (s, 1H), 8.31 (s, 1H), 8.22 (d, |
| | | | | J = 8.1 Hz, 1H), 7.74 (t, J = 7.8 Hz, 1H), |
| | | | | 7.55 (s, 1H), 7.44 (d, J = 8.1 Hz, 1H), 3.42 |
| | | | | (m, 2H), 2.76 (m, 2H), 2.03 (m, 1H), 1.74 |
| | | | | (m, 2H), 1.32 (rn, 2H), 1.08 (m, 2H). |
| 2-90 | 3.70 | 463.2 | 2 | CDCl₃ δ 8.47 (s, 1H), 8.29 (dd, J = 8.3, 1.0 |
| | | | | Hz, 1H), 8.07 (d, J = 9.4 Hz, 1H), 7.57 (t, |
| | | | | J = 8.3 Hz, 1H), 7.21 (d, J = 8.0 Hz, 1H), |
| | | | | 6.84 (d, J = 9.4 Hz, 1H), 4.14 (m, 2H), 3.82 |
| | | | | (s, 2H), 3.47 (m, 2H), 2.42 (m, 4H), 2.29 (s, |
| | | | | 2H), 2.12 (m, 2H), 1.60 (m, 2H), 1.07 (t, J = |
| | | | | 6.7 Hz, 3H). |
| 2-91 | 4.04 | 495.3 | 2 | CDCl₃ δ 8.46 (m, 1H), 8.26 (m, 1H), 8.13 |
| | | | | (m, 1H), 7.57 (m, 1H), 7.17 (m, 1H), 5.66 |
| | | | | (m, 1H), 3.63 (m, 2H), 2.68 (m, 6H), 2.04 |
| | | | | (m, 2H), 1.92 (m, 4H), 1.61 (m, 3H), 1.26 |
| | | | | (m, 3H). |
| 2-92 | 3.51 | 423.2 | 2 | MeOD δ 8.48 (s, 1H), 8.30 (d, J = 7.9 Hz, |
| | | | | 1H), 7.68 (t, J = 7.5 Hz, 1H), 7.35 (d, J = |
| | | | | 7.4 Hz, 1H), 6.09 (s, 1H), 4.05 (s, 3H), 3.44 |
| | | | | (m, 4H), 3.02 (m, 2H), 2.06 (m, 3H), 1.50 |
| | | | | (m, 2H). |
| 2-93 | 5.28 | 369.2 | 1 | ¹H NMR (300 MHz, CDCl₃) δ 7.70 (m, 1H), |
| | | | | 7.60 (m, 1H), 7.39 (m, 1H), 6.77 (m, 1H), |
| | | | | 5.82 (s, 1H), 4.74 (m, 1H), 4.14 (m, 4H), |
| | | | | 4.04 (m, 2H), 3.55 (m, 2H), 3.07 (s, 6H), |
| | | | | 2.12 (m, 2H), 1.61 (m, 2H). |
| 2-94 | 3.34 | 422.3 | 1 | DMSO δ 7.84 (m, 1H), 7.54 (m, 1H), 7.44 |
| | | | | (m, 1H), 7.36 (m, 1H), 6.76 (dd, J = 8.1, 1.9 |
| | | | | Hz, 1H), 6.12 (s, 1H), 3.99 (s, 3H), 3.44 (m, |
| | | | | 2H), 3.00 (s, 6H), 2.93 (m, 2H), 2.84 (m, |
| | | | | 2H), 2.55 (m, 1H), 1.92 (m, 2H), 1.70 (m, |
| | | | | 2H), 1.63 (m, 2H), 1.54 (m, 2H). |
| 2-95 | 3.04 | 379.2 | 1 | MeOD δ 9.09 (d, J = 1.6 Hz, 1H), 8.90 (d, |
| | | | | J = 5.5 Hz, 1H), 8.54 (dd, J = 5.5, 1.6 Hz, |
| | | | | 1H), 8.01 (d, J = 9.1 Hz, 1H), 6.73 (d, J = |
| | | | | 9.1 Hz, 1H), 3.50 (m, 1H), 3.45 (m, 2H), |
| | | | | 3.00 (m, 2H), 2.19 (m, 1H), 2.10 (m, 2H), |
| | | | | 1.53 (m, 2H). |
| 2-96 | 3.05 | 408.3 | 1 | DMSO δ 8.82 (broad s, 2H), 7.91 (broad s, |
| | | | | 1H), 7.55 (m, 1H), 7.42 (m, 1H), 6.91 (m, |
| | | | | 1H), 6.09 (s, 1H), 4.42 (m, 1H), 4.00 (s, |
| | | | | 3H), 3.04 (m, 8H), 2.93 (m, 2H), 2.36 (m, |
| | | | | 2H), 1.75 (m, 6H). |

### Example 69

### Analytical Data

### PIM-1, PIM-2 and PIM-3 Activity and Flt3 Activity

Biological activity in PIM-1, PIM-2, PIM-3 and/or Flt3 for certain examples is represented in Table 3 by semi-quantitative results: IC50>1 μM (+), IC50 <100 nM (+++), 100 nM<IC50<1 μM (++). There is also some quantitative data, depicted in parentheses, which depict the actual IC₅₀ values for representative examples.

TABLE 3

| | | PIM1 IC₅₀ | PIM2 IC₅₀ | PIM3 IC₅₀ | FLT3 IC₅₀ |
|---|---|---|---|---|---|
| No. | | (nM) | (nM) | (nM) | (nM) |
| 2-01 | 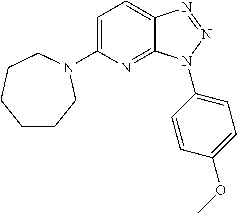 [SMILES: COc1ccc(-n2nnc3ccc(N4CCCCCC4)nc32)cc1] | + | + | | |
| 2-02 | 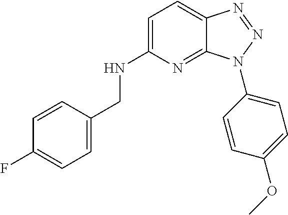 [SMILES: COc1ccc(-n2nnc3ccc(NCc4ccc(F)cc4)nc32)cc1] | + | + | | |
| 2-03 | 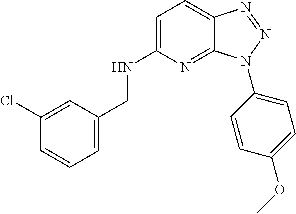 [SMILES: COc1ccc(-n2nnc3ccc(NCc4cccc(Cl)c4)nc32)cc1] | + | + | | |
| 2-04 | 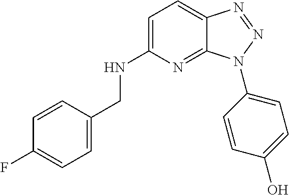 [SMILES: Oc1ccc(-n2nnc3ccc(NCc4ccc(F)cc4)nc32)cc1] | +++ (69) | ++ | +++ (51) | + |
| 2-05 | 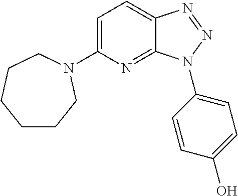 [SMILES: Oc1ccc(-n2nnc3ccc(N4CCCCCC4)nc32)cc1] | ++ | | | + |
| 2-06 | 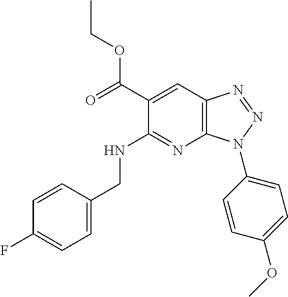 [SMILES: CCOC(=O)c1cc2nnn(-c3ccc(OC)cc3)c2nc1NCc1ccc(F)cc1] | + | + | | |
| 2-07 | 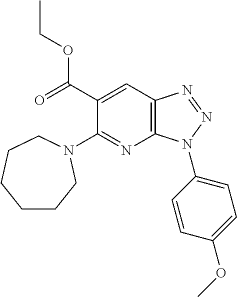 [SMILES: CCOC(=O)c1cc2nnn(-c3ccc(OC)cc3)c2nc1N1CCCCCC1] | + | + | | |
| 2-08 | 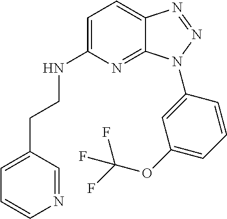 [SMILES: FC(F)(F)Oc1cccc(-n2nnc3ccc(NCCc4cccnc4)nc32)c1] | ++ | + | | |
| 2-09 | 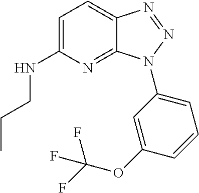 [SMILES: CCCNc1ccc2nnn(-c3cccc(OC(F)(F)F)c3)c2n1] | ++ | + | | |
| 2-10 | 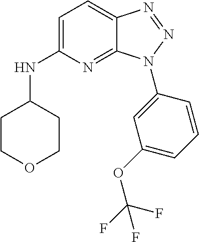 [SMILES: FC(F)(F)Oc1cccc(-n2nnc3ccc(NC4CCOCC4)nc32)c1] | ++ | + | | |
| 2-11 | 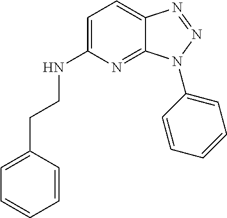 [SMILES: c1ccc(CCNc2ccc3nnn(-c4ccccc4)c3n2)cc1] | ++ | + | | |
| 2-12 | 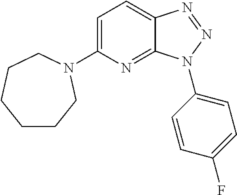 [SMILES: Fc1ccc(-n2nnc3ccc(N4CCCCCC4)nc32)cc1] | + | + | | |
| 2-13 | 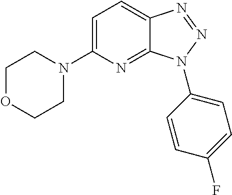 [SMILES: Fc1ccc(-n2nnc3ccc(N4CCOCC4)nc32)cc1] | + | + | | |
| 2-14 | 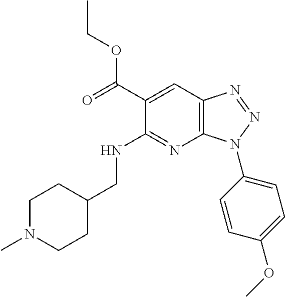 [SMILES: CCOC(=O)c1cc2nnn(-c3ccc(OC)cc3)c2nc1NCC1CCN(C)CC1] | + | + | | |
| 2-15 | 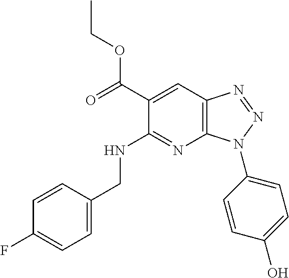 [SMILES: CCOC(=O)c1cc2nnn(-c3ccc(O)cc3)c2nc1NCc1ccc(F)cc1] | + | + | | |
| 2-16 | 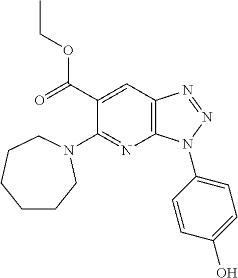 [SMILES: CCOC(=O)c1cc2nnn(-c3ccc(O)cc3)c2nc1N1CCCCCC1] | + | + | | |
| 2-17 | 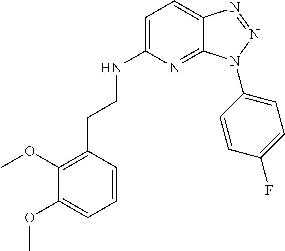 [SMILES: COc1cccc(CCNc2ccc3nnn(-c4ccc(F)cc4)c3n2)c1OC] | + | + | | |
| 2-18 | 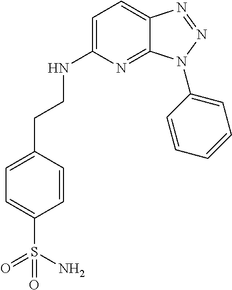 [SMILES: NS(=O)(=O)c1ccc(CCNc2ccc3nnn(-c4ccccc4)c3n2)cc1] | ++ | + | | |
| 2-19 | 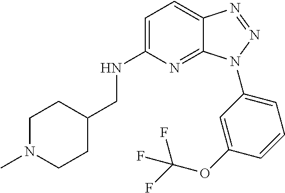 [SMILES: CN1CCC(CNc2ccc3nnn(-c4cccc(OC(F)(F)F)c4)c3n2)CC1] | ++ | + | ++ | + |
| 2-20 | 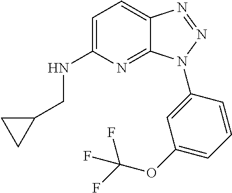 [SMILES: FC(F)(F)Oc1cccc(-n2nnc3ccc(NCC4CC4)nc32)c1] | ++ | + | | |
| 2-21 | 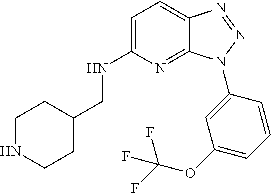 [SMILES: FC(F)(F)Oc1cccc(-n2nnc3ccc(NCC4CCNCC4)nc32)c1] | +++ (6) | ++ | +++ (7) | ++ |
| 2-22 | 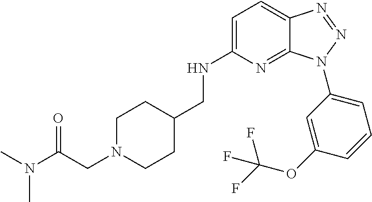 [SMILES: CN(C)C(=O)CN1CCC(CNc2ccc3nnn(-c4cccc(OC(F)(F)F)c4)c3n2)CC1] | ++ | + | | |
| 2-23 | 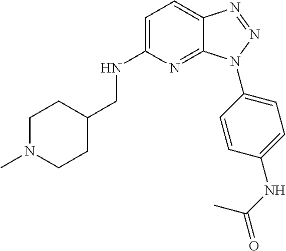 [SMILES: CC(=O)Nc1ccc(-n2nnc3ccc(NCC4CCN(C)CC4)nc32)cc1] | + | + | | |
| 2-24 | 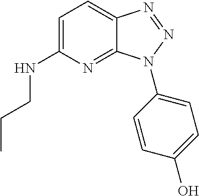 [SMILES: CCCNc1ccc2nnn(-c3ccc(O)cc3)c2n1] | ++ | + | | |
| 2-25 | 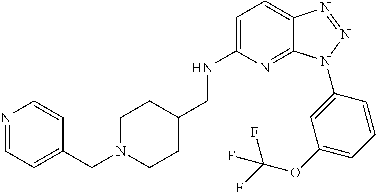 [SMILES: FC(F)(F)Oc1cccc(-n2nnc3ccc(NCC4CCN(Cc5ccncc5)CC4)nc32)c1] | +++ (34) | + | +++ (22) | + |
| 2-26 | 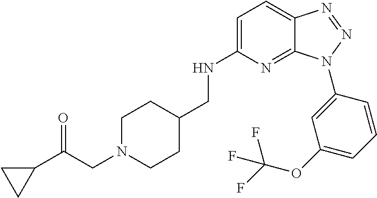 [SMILES: O=C(CN1CCC(CNc2ccc3nnn(-c4cccc(OC(F)(F)F)c4)c3n2)CC1)C1CC1] | ++ | + | | |
| 2-27 | 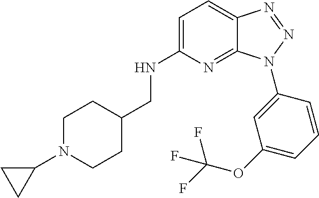 [SMILES: FC(F)(F)Oc1cccc(-n2nnc3ccc(NCC4CCN(C5CC5)CC4)nc32)c1] | ++ | + | | |
| 2-28 | 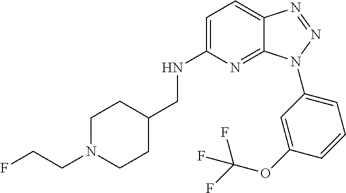 [SMILES: FCCN1CCC(CNc2ccc3nnn(-c4cccc(OC(F)(F)F)c4)c3n2)CC1] | ++ | + | + | + |
| 2-29 | 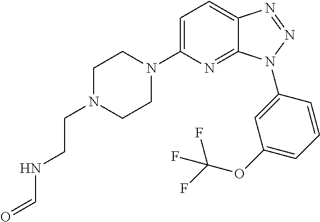 [SMILES: O=CNCCN1CCN(c2ccc3nnn(-c4cccc(OC(F)(F)F)c4)c3n2)CC1] | ++ | + | | |
| 2-30 | 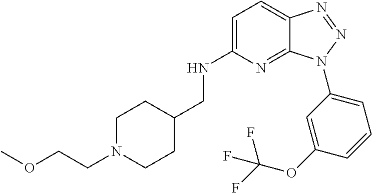 [SMILES: COCCN1CCC(CNc2ccc3nnn(-c4cccc(OC(F)(F)F)c4)c3n2)CC1] | ++ | + | | + |
| 2-31 | 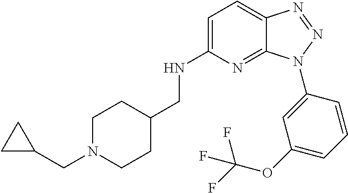 [SMILES: FC(F)(F)Oc1cccc(-n2nnc3ccc(NCC4CCN(CC5CC5)CC4)nc32)c1] | ++ | + | | + |
| 2-32 | 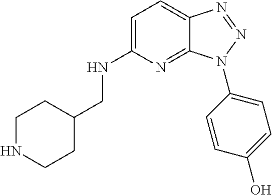 [SMILES: Oc1ccc(-n2nnc3ccc(NCC4CCNCC4)nc32)cc1] | +++ (11) | + | +++ (15) | ++ |
| 2-33 | 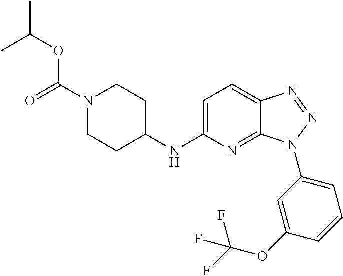 [SMILES: CC(C)OC(=O)N1CCC(Nc2ccc3nnn(-c4cccc(OC(F)(F)F)c4)c3n2)CC1] | + | + | | |
| 2-34 | 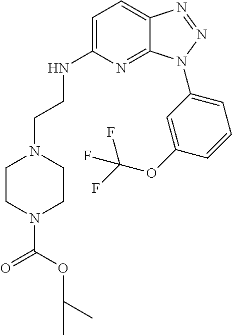 [SMILES: CC(C)OC(=O)N1CCN(CCNc2ccc3nnn(-c4cccc(OC(F)(F)F)c4)c3n2)CC1] | + | + | | |
| 2-35 | 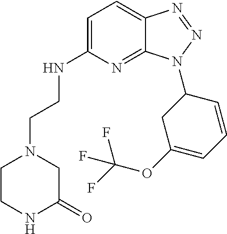 [SMILES: O=C1CN(CCNc2ccc3nnn(C4C=CC=C(OC(F)(F)F)C4)c3n2)CCN1] | ++ | + | | |
| 2-36 | 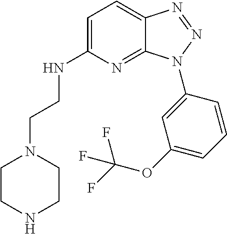 [SMILES: FC(F)(F)Oc1cccc(-n2nnc3ccc(NCCN4CCNCC4)nc32)c1] | ++ | + | | ++ |
| 2-37 | 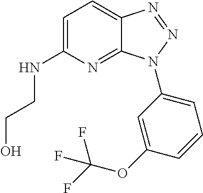 [SMILES: OCCNc1ccc2nnn(-c3cccc(OC(F)(F)F)c3)c2n1] | ++ | + | | |
| 2-38 | 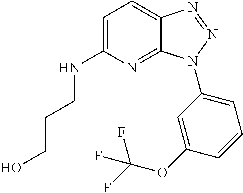 [SMILES: OCCCNc1ccc2nnn(-c3cccc(OC(F)(F)F)c3)c2n1] | ++ | + | | + |
| 2-39 | 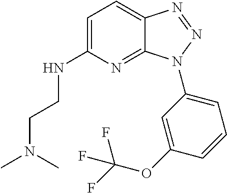 [SMILES: CN(C)CCNc1ccc2nnn(-c3cccc(OC(F)(F)F)c3)c2n1] | + | + | | |
| 2-40 | 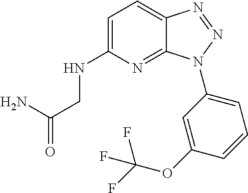 [SMILES: NC(=O)CNc1ccc2nnn(-c3cccc(OC(F)(F)F)c3)c2n1] | + | + | | |
| 2-41 | 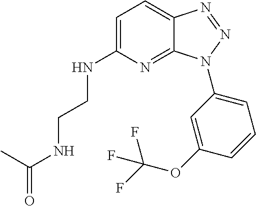 [SMILES: CC(=O)NCCNc1ccc2nnn(-c3cccc(OC(F)(F)F)c3)c2n1] | + | + | | |
| 2-42 | 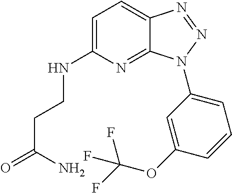 [SMILES: NC(=O)CCNc1ccc2nnn(-c3cccc(OC(F)(F)F)c3)c2n1] | ++ | + | | |
| 2-43 | 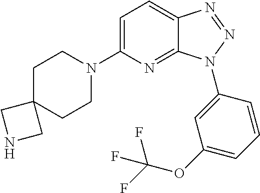 [SMILES: FC(F)(F)Oc1cccc(-n2nnc3ccc(N4CCC5(CC4)CNC5)nc32)c1] | ++ | + | | + |
| 2-44 | 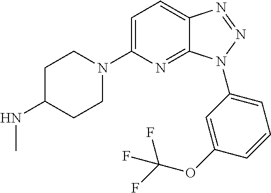 [SMILES: CNC1CCN(c2ccc3nnn(-c4cccc(OC(F)(F)F)c4)c3n2)CC1] | +++ (87) | + | ++ | + |
| 2-45 | 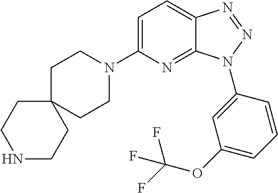 [SMILES: FC(F)(F)Oc1cccc(-n2nnc3ccc(N4CCC5(CCNCC5)CC4)nc32)c1] | ++ | + | | + |
| 2-46 | 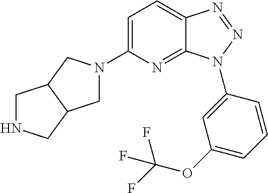 [SMILES: FC(F)(F)Oc1cccc(-n2nnc3ccc(N4CC5CNCC5C4)nc32)c1] | ++ | + | | + |
| 2-47 | 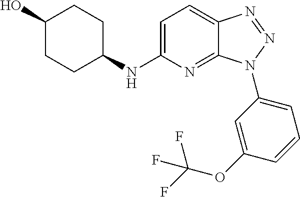 [SMILES: O[C@H]1CC[C@@H](Nc2ccc3nnn(-c4cccc(OC(F)(F)F)c4)c3n2)CC1] | +++ (26) | ++ | +++ (84) | + |
| 2-48 | 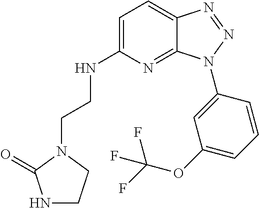 [SMILES: O=C1NCCN1CCNc1ccc2nnn(-c3cccc(OC(F)(F)F)c3)c2n1] | ++ | + | | |
| 2-49 | 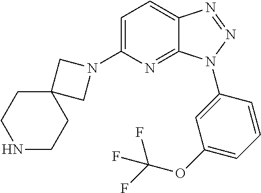 [SMILES: FC(F)(F)Oc1cccc(-n2nnc3ccc(N4CC5(CCNCC5)C4)nc32)c1] | +++ (10) | ++ | ++ | + |
| 2-50 | 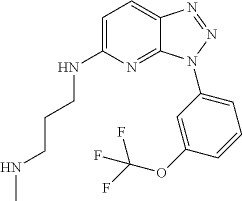 [SMILES: CNCCCNc1ccc2nnn(-c3cccc(OC(F)(F)F)c3)c2n1] | ++ | + | | |
| 2-51 | 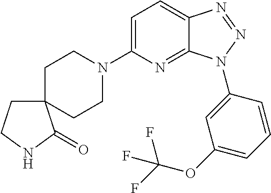 [SMILES: O=C1NCCC12CCN(c1ccc3nnn(-c4cccc(OC(F)(F)F)c4)c3n1)CC2] | + | + | | |
| 2-52 | 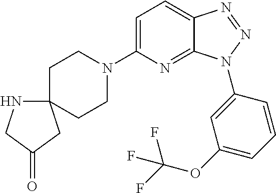 [SMILES: O=C1CNC2(CCN(c3ccc4nnn(-c5cccc(OC(F)(F)F)c5)c4n3)CC2)C1] | + | + | | |
| 2-53 | 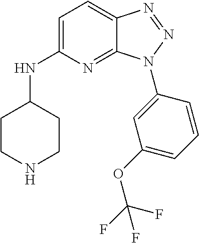 [SMILES: FC(F)(F)Oc1cccc(-n2nnc3ccc(NC4CCNCC4)nc32)c1] | +++ (38) | ++ | +++ (66) | + |
| 2-54 | 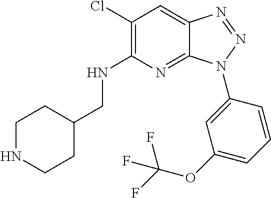 [SMILES: FC(F)(F)Oc1cccc(-n2nnc3cc(Cl)c(NCC4CCNCC4)nc32)c1] | +++ (7) | + | +++ (16) | ++ |
| 2-55 | 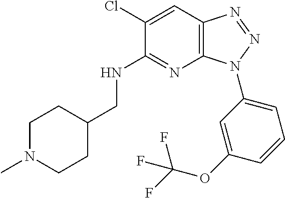 [SMILES: CN1CCC(CNc2nc3c(cc2Cl)nnn3-c2cccc(OC(F)(F)F)c2)CC1] | +++ (90) | + | + | ++ |
| 2-56 | 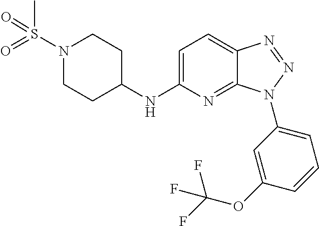 [SMILES: CS(=O)(=O)N1CCC(Nc2ccc3nnn(-c4cccc(OC(F)(F)F)c4)c3n2)CC1] | +++ (79) | ++ | + | + |
| 2-57 | 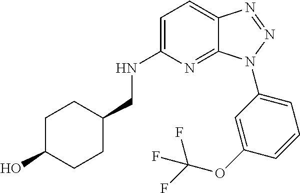 [SMILES: O[C@H]1CC[C@@H](CNc2ccc3nnn(-c4cccc(OC(F)(F)F)c4)c3n2)CC1] | +++ | + | ++ | + |
| 2-58 | 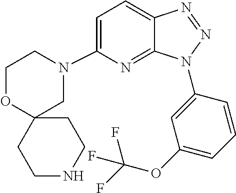 [SMILES: FC(F)(F)Oc1cccc(-n2nnc3ccc(N4CCOC5(CCNCC5)C4)nc32)c1] | +++ (85) | + | + | + |
| 2-59 | 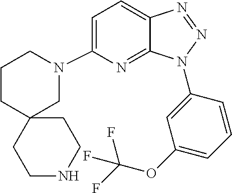 [SMILES: FC(F)(F)Oc1cccc(-n2nnc3ccc(N4CCCC5(CCNCC5)C4)nc32)c1] | +++ | + | ++ | + |
| 2-60 | 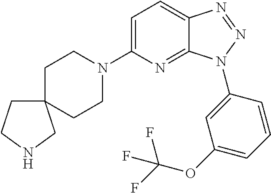 [SMILES: FC(F)(F)Oc1cccc(-n2nnc3ccc(N4CCC5(CCNC5)CC4)nc32)c1] | ++ | + | | + |
| 2-61 | 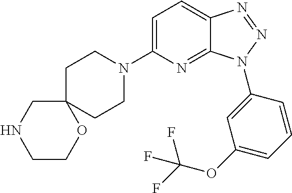 [SMILES: FC(F)(F)Oc1cccc(-n2nnc3ccc(N4CCC5(CC4)CNCCO5)nc32)c1] | +++ (74) | + | + | + |
| 2-62 | 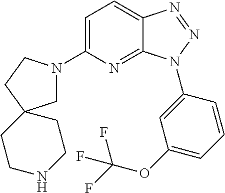 [SMILES: FC(F)(F)Oc1cccc(-n2nnc3ccc(N4CCC5(CCNCC5)C4)nc32)c1] | +++ (68) | + | ++ | + |
| 2-63 | 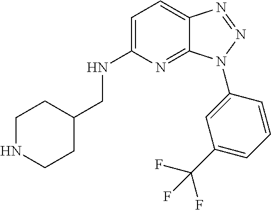 [SMILES: FC(F)(F)c1cccc(-n2nnc3ccc(NCC4CCNCC4)nc32)c1] | +++ | + | ++ | + |
| 2-64 | 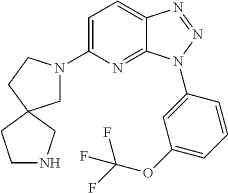 [SMILES: FC(F)(F)Oc1cccc(-n2nnc3ccc(N4CCC5(CCNC5)C4)nc32)c1] | ++ | + | | + |
| 2-65 | 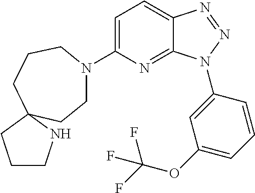 [SMILES: FC(F)(F)Oc1cccc(-n2nnc3ccc(N4CCCC5(CCCN5)CC4)nc32)c1] | +++ (2) | ++ | +++ (6) | +++ (28) |
| 2-66 | 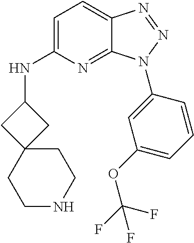 [SMILES: FC(F)(F)Oc1cccc(-n2nnc3ccc(NC4CC5(CCNCC5)C4)nc32)c1] | +++ (11) | ++ | +++ (23) | ++ |
| 2-67 | 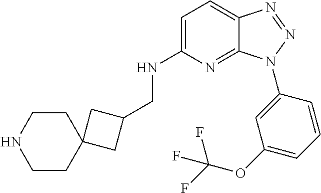 [SMILES: FC(F)(F)Oc1cccc(-n2nnc3ccc(NCC4CC5(CCNCC5)C4)nc32)c1] | +++ (4.7) | ++ | +++ (40) | +++ (12) |
| 2-68 | 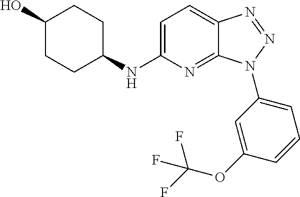 [SMILES: O[C@H]1CC[C@@H](Nc2ccc3nnn(-c4cccc(OC(F)(F)F)c4)c3n2)CC1] | +++ (14) | +++ (69) | +++ (56) | |
| 2-69 | 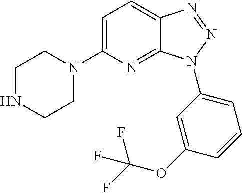 [SMILES: FC(F)(F)Oc1cccc(-n2nnc3ccc(N4CCNCC4)nc32)c1] | ++ | + | ++ | + |
| 2-70 | 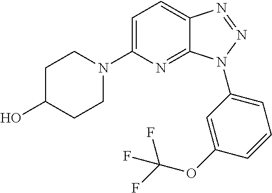 [SMILES: OC1CCN(c2ccc3nnn(-c4cccc(OC(F)(F)F)c4)c3n2)CC1] | ++ | ++ | ++ | + |
| 2-71 | 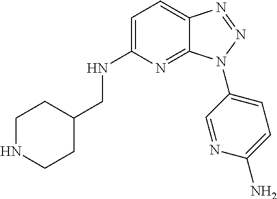 [SMILES: Nc1ccc(-n2nnc3ccc(NCC4CCNCC4)nc32)cn1] | ++ | + | | ++ |
| 2-72 | 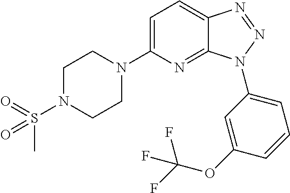 [SMILES: CS(=O)(=O)N1CCN(c2ccc3nnn(-c4cccc(OC(F)(F)F)c4)c3n2)CC1] | ++ | + | + | |
| 2-73 | 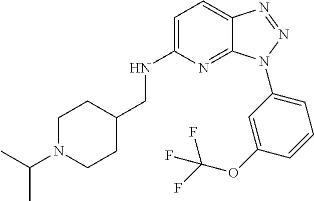 [SMILES: CC(C)N1CCC(CNc2ccc3nnn(-c4cccc(OC(F)(F)F)c4)c3n2)CC1] | +++ (77) | + | ++ | + |
| 2-74 | 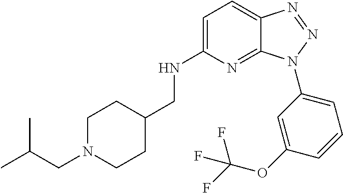 [SMILES: CC(C)CN1CCC(CNc2ccc3nnn(-c4cccc(OC(F)(F)F)c4)c3n2)CC1] | ++ | + | | + |
| 2-75 | 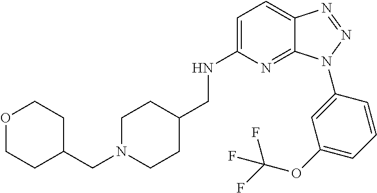 [SMILES: FC(F)(F)Oc1cccc(-n2nnc3ccc(NCC4CCN(CC5CCOCC5)CC4)nc32)c1] | ++ | + | | + |
| 2-76 | 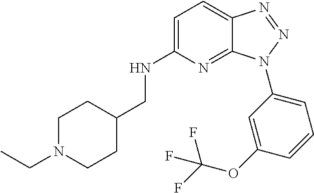 [SMILES: CCN1CCC(CNc2ccc3nnn(-c4cccc(OC(F)(F)F)c4)c3n2)CC1] | +++ (35) | + | ++ | + |
| 2-77 | 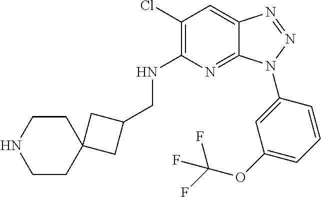 [SMILES: FC(F)(F)Oc1cccc(-n2nnc3cc(Cl)c(NCC4CC5(CCNCC5)C4)nc32)c1] | ++ | + | | + |
| 2-78 | 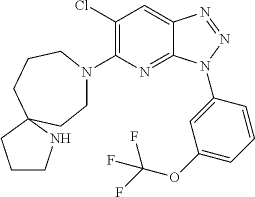 [SMILES: FC(F)(F)Oc1cccc(-n2nnc3cc(Cl)c(N4CCCC5(CCCN5)CC4)nc32)c1] | +++ | + | +++ (56) | +++ (20) |
| 2-79 | 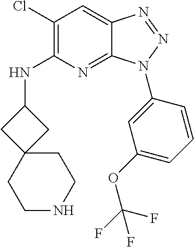 [SMILES: FC(F)(F)Oc1cccc(-n2nnc3cc(Cl)c(NC4CC5(CCNCC5)C4)nc32)c1] | +++ (33) | + | +++ (40) | ++ |
| 2-80 | 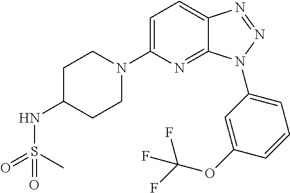 [SMILES: CS(=O)(=O)NC1CCN(c2ccc3nnn(-c4cccc(OC(F)(F)F)c4)c3n2)CC1] | ++ | + | ++ | + |
| 2-81 | 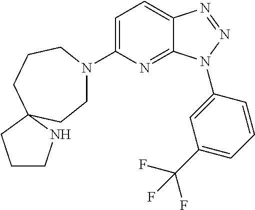 [SMILES: FC(F)(F)c1cccc(-n2nnc3ccc(N4CCCC5(CCCN5)CC4)nc32)c1] | +++ (71) | + | ++ | ++ |
| 2-82 | 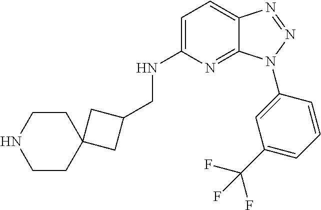 [SMILES: FC(F)(F)c1cccc(-n2nnc3ccc(NCC4CC5(CCNCC5)C4)nc32)c1] | +++ (9) | ++ | +++ (28) | +++ (28) |
| 2-83 | 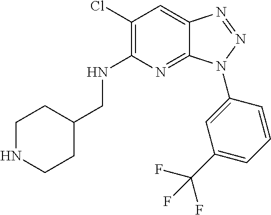 [SMILES: FC(F)(F)c1cccc(-n2nnc3cc(Cl)c(NCC4CCNCC4)nc32)c1] | +++ (21) | + | +++ (6) | +++ (94) |
| 2-84 | 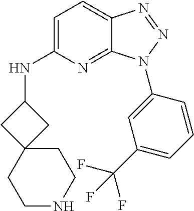 [SMILES: FC(F)(F)c1cccc(-n2nnc3ccc(NC4CC5(CCNCC5)C4)nc32)c1] | +++ (14) | ++ | +++ (19) | ++ |
| 2-85 | 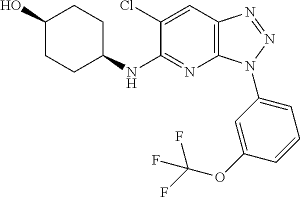 [SMILES: O[C@H]1CC[C@@H](Nc2nc3c(cc2Cl)nnn3-c2cccc(OC(F)(F)F)c2)CC1] | +++ (74) | ++ | ++ | ++ |
| 2-86 | 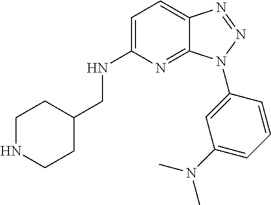 [SMILES: CN(C)c1cccc(-n2nnc3ccc(NCC4CCNCC4)nc32)c1] | +++ (5) | + | +++ (11) | + |
| 2-87 | 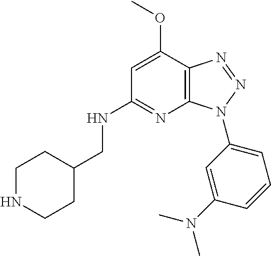 [SMILES: COc1cc(NCC2CCNCC2)nc2c1nnn2-c1cccc(N(C)C)c1] | +++ (1.6) | ++ | +++ (2.4) | + |
| 2-88 | 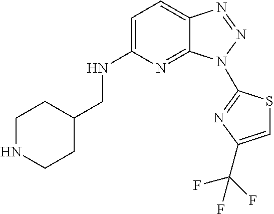 [SMILES: FC(F)(F)c1csc(-n2nnc3ccc(NCC4CCNCC4)nc32)n1] | ++ | + | | + |
| 2-89 | 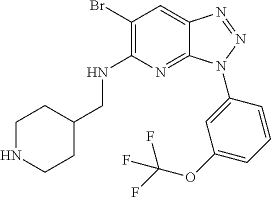 [SMILES: FC(F)(F)Oc1cccc(-n2nnc3cc(Br)c(NCC4CCNCC4)nc32)c1] | +++ (11) | ++ | +++ (8) | +++ (58) |
| 2-90 | 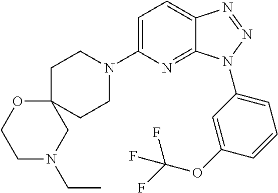 [SMILES: CCN1CCOC2(CCN(c3ccc4nnn(-c5cccc(OC(F)(F)F)c5)c4n3)CC2)C1] | + | + | | ++ |
| 2-91 | 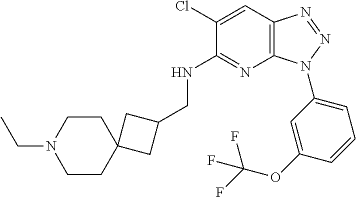 [SMILES: CCN1CCC2(CC1)CC(CNc1nc3c(cc1Cl)nnn3-c1cccc(OC(F)(F)F)c1)C2] | ++ | + | | ++ |
| 2-92 | 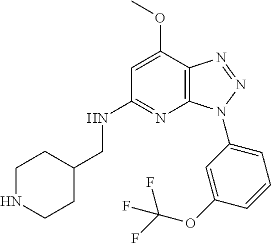 [SMILES: COc1cc(NCC2CCNCC2)nc2c1nnn2-c1cccc(OC(F)(F)F)c1] | +++ (0.9) | +++ (29) | +++ (0.7) | + |
| 2-93 | 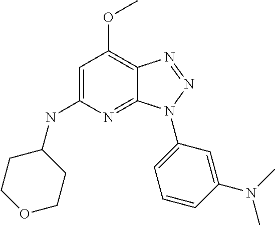 [SMILES: COc1cc(NC2CCOCC2)nc2c1nnn2-c1cccc(N(C)C)c1] | ++ | | | + |
| 2-94 | 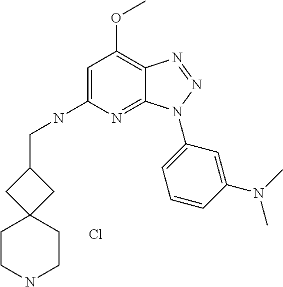 [SMILES: COc1cc(NCC2CC3(CCNCC3)C2)nc2c1nnn2-c1cccc(N(C)C)c1 ; Cl] | +++ (9) | + | +++ (56) | + |
| 2-95 | 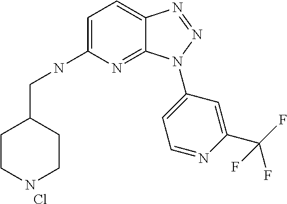 [SMILES: FC(F)(F)c1cc(-n2nnc3ccc(NCC4CCNCC4)nc32)ccn1 ; Cl] | ++ | | | |
| 2-96 | 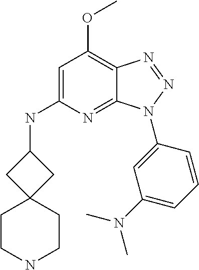 [SMILES: COc1cc(NC2CC3(CCNCC3)C2)nc2c1nnn2-c1cccc(N(C)C)c1] | +++ (32) | + | +++ (68) | + |

### Example 70

The following table demonstrates that representative compounds of the examples:
(i) inhibit PIM-1 in the cellular assay described hereinbefore;
(ii) display metabolic stability in human liver microsomes; and
(iii) may be selective inhibitors, as described hereinbefore and as may be demonstrated by the low percentage inhibitions of certain other kinases.

### Table 4:

Data for some representative compounds (2-92, 2-87, 2-65, 2-67, 2-86, 2-21, 2-54, 2-66, 2-47 and 2-83) in the cellular assay (inhibition of Bad-phosphorylation; see hereinbefore), for metabolic stability in human liver microsomes (shown in the table as percentage metabolic stability) and for percentage of inhibition in a panel of 24 kinases at 1 μM.

TABLE 4

| | Cell assay | c | | | | | | | | | | | |
|---|---|---|---|---|---|---|---|---|---|---|---|---|---|
| | BAD_P | Stability | proquinase, % inhibiton at 1 uM | | | | | | | | | | |
| | H1299 | in | | B_RAF | | CK1 | CDK8 | DYRK | | | | | IKK |
| Cpd | PIM1 | HLM | AKT2 | V600E | CHK1 | Alpha 1 | CYCC | 1A | EGF_R | FAK | FGFR1 | IGF1_R | BETA |
| 2-92 | 1.36E−07 | 96.65 | 0 | 1 | 0 | 4 | 0 | 0 | 1 | 0 | 0 | 5 | 0 |
| 2-87 | 1.00E−07 | 86.25 | 4 | 0 | 3 | 5 | 0 | 1 | 0 | 6 | 6 | 0 | 0 |
| 2-65 | | 88.9 | 0 | 3 | 7 | 0 | 2 | 12 | 12 | 31 | 22 | 21 | 0 |
| 2-67 | | 92.15 | 0 | 11 | 11 | 0 | 0 | 18 | 13 | 27 | 0 | 25 | 0 |
| 2-85 | 2.62E−07 | | | | | | | | | | | | |
| 2-21 | 3.06E−07 | 97.95 | | | | | | | | | | | |
| 2-54 | 5.99E−07 | 93.95 | | 7 | | 6 | | 30 | 18 | 28 | 32 | 26 | 17 |
| 2-66 | | 91.1 | 0 | 15 | 13 | 0 | 3 | 30 | 18 | 19 | 4 | 16 | 5 |
| 2-47 | 4.05E−07 | 43.6 | | 6 | | 2 | | 3 | 9 | 6 | 0 | 8 | 4 |
| 2-83 | 4.58E−07 | 112.7 | 0 | 11 | 6 | 3 | 14 | 7 | 8 | 16 | 39 | 13 | 0 |

| | proquinase, % inhibiton at 1 uM | | | | | | | | | | | | |
|---|---|---|---|---|---|---|---|---|---|---|---|---|---|
| | | | | | | | PDGFR | | | RPS5 | | | VEGF |
| Cpd | INS_R | JAK2 | KIT | MEK1 | MET | PAK1 | Alpha | PDK1 | PLK1 | KA1 | SGK1 | SRC | R2 |
| 2-92 | 0 | 2 | 2 | 1 | 0 | 8 | 0 | 0 | 0 | 7 | 6 | 8 | 0 |
| 2-87 | 0 | 1 | 10 | 7 | 0 | 10 | 0 | 9 | 2 | 12 | 9 | 0 | 0 |
| 2-65 | 12 | 8 | 63 | 13 | 11 | 0 | 57 | 0 | | 10 | 0 | 33 | 10 |
| 2-67 | 5 | 0 | 58 | 0 | 2 | 3 | 49 | 0 | | 11 | 7 | 16 | 0 |
| 2-85 | | | | | | | | | | | | | |
| 2-21 | | | | | | | | | | | | | |
| 2-54 | 16 | 15 | 34 | 0 | 19 | | 23 | | | 8 | 7 | | |
| 2-66 | 7 | 3 | 30 | 0 | 3 | 11 | 12 | 4 | | 19 | 2 | 28 | 22 |
| 2-47 | 7 | 15 | 2 | 0 | 0 | | 0 | | | 0 | 3 | | |
| 2-83 | 6 | 1 | 26 | 2 | 0 | 11 | 14 | 3 | 0 | 36 | 6 | 24 | 34 |

### Example 71

Representative compounds of the examples were shown to display a synergistic effect when combined with other therapeutic agents and tested in certain cell lines, as is demonstrated by Table 5.

TABLE 5
Combination assays

| | | | ChemoEC50 | Chemo | | | |
|---|---|---|---|---|---|---|---|
| | | | Example | EC50 | Pim Inh | Combination | |
| Cell line | Tumor type | Chemotherapeutic | number | [mM] | EC50 | Index (CI) | Synergy |
| MiaPaca-2 | pancreas | lapatinib | 2-21 | 20 | 8 | 0.346 | ++ |
| MiaPaca-2 | pancreas | Gemcitabine | 2-92 | 0.05 | 3 | 0.786 | + |
| MV4:11 | leukemia (AML) | GDC-0941 | 2-68 | 0.5 | 15 | 0.41 | ++ |
| MV4:11 | leukemia (AML) | GDC-0941 | 2-25 | 0.5 | 1.5 | 0.311 | ++ |
| MV4:11 | leukemia (AML) | GDC-0941 | 2-55 | 0.5 | 1 | 0.35 | ++ |
| MV4:11 | leukemia (AML) | lapatinib | 2-92 | 5 | 5 | 0.312 | ++ |
| Jeko-1 | Mantle cell | GDC-0941 | 2-21 | 1 | 1.5 | 0.23 | ++ |
| | lymphoma | | | | | | |
| Jeko-1 | Mantle cell | lapatinib | 2-25 | 5 | 9 | 0.387 | ++ |
| | lymphoma | | | | | | |
| Jeko-1 | Mantle cell | lapatinib | 2-68 | 10 | 10 | 0.35 | ++ |
| | lymphoma | | | | | | |
| Jeko-1 | Mantle cell | lapatinib | 2-55 | 5 | 3.75 | 0.15 | +++ |
| | lymphoma | | | | | | |
| SKMel19 | melanoma | GDC-0879 | 2-25 | 0.3 | 5.875 | 0.87 | + |
| SKMel19 | melanoma | GDC-0879 | 2-92 | 0.3 | 1 | 0.18 | +++ |
| SKMel19 | melanoma | GDC-0941 | 2-92 | 0.5 | 1 | 0.277 | ++ |
| DU145 | prostate | Taxotere | 2-92 | 0.25 | 7 | 0.627 | ++ |
| A549 | lung | GDC-0941 | 2-92 | 6 | 10 | 0.183 | +++ |
| | adenocarcinoma | | | | | | |
| HTC116 | colon carcinoma | PD-0325901 | 2-92 | 1.25 | 10 | 0.266 | +++ |
| HTC116 | colon carcinoma | lapatinib | 2-92 | 10 | 10 | 0.223 | +++ |
| NCIH1975 | non small cell lung | GDC-0941 | 2-92 | 20 | 10 | 0.553 | ++ |
| | carcinoma | | | | | | |
| PC3 | prostate cancer | GDC-0941 | 2-92 | 10 | 1 | 0.843 | + |
| NCIH23 | non small cell lung | lapatinib | 2-54 | 10 | 5 | 0.15 | +++ |
| | carcinoma | | | | | | |
| NCIH23 | non small cell lung | lapatinib | 2-54 | 10 | 5 | 0.732 | + |
| | carcinoma | | | | | | |
| NCIH23 | non small cell lung | lapatinib | 2-49 | 10 | 10 | 0.785 | + |
| | carcinoma | | | | | | |

Combination index (CI) calculated for the combination of compounds of the invention and various chemotherapeutic agents in the MTT in vitro cell proliferation assays:
CI < 0.1 (++++),
0.1 < CI < 0.3 (+++),
0.3 < CI < 0.7 (++),
0.7 < CI < 1.2 (+)

1. A compound of formula I, 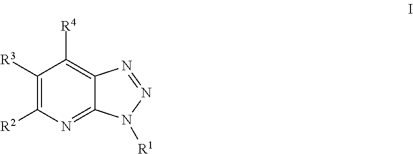
wherein:
R¹ represents aryl or heteroaryl, both of which are optionally substituted by one or more substituents selected from E¹;
R² represents a fragment of formula IA, 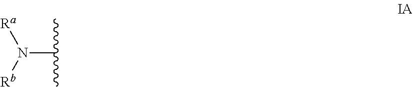
wherein R^{a} and R^{b} independently represent H, —C(O)—C₁₋₁₁ alkyl, —S(O)₂—C₁₋₁₁ alkyl, C₁₋₁₂ alkyl, heterocycloalkyl (which latter four groups are optionally substituted by one or more substituents selected from ═O, ═NOR^{7a} and Q¹), aryl or heteroaryl (which latter two groups are optionally substituted by one or more substituents selected from Q²); or
R^{a} and R^{b} are linked together, along with the requisite nitrogen atom to which they are necessarily attached, to form a (first) 3- to 7-membered cyclic group, optionally containing one further heteroatom selected from nitrogen, sulfur and oxygen, and which ring optionally:
(a) is fused to a second ring that is either a 3- to 7-membered saturated heterocycloalkyl group containing one to four heteroatoms selected from oxygen, sulfur and nitrogen, a 3- to 12-membered saturated carbocyclic ring, or an unsaturated 5- to 12-membered carbocyclic or heterocyclic ring;
(b) comprises a linker group —(C(R^{x})₂)ₚ— and/or —(C(R^{x})₂)ᵣ—O—(C(R^{x})₂)ₛ— (wherein p is 1 or 2; r is 0 or 1; s is 0 or 1; and each R^{x} independently represents hydrogen or C₁₋₆ alkyl), linking together any two non-adjacent atoms of the first 3- to 7-membered ring; or
(c) comprises a second ring that is either a 3- to 12-membered saturated carbocyclic ring or a 3- to 7-membered saturated heterocycloalkyl group containing one to four heteroatoms selected from oxygen and nitrogen, and which second ring is linked together with the first ring via a single carbon atom common to both rings,
all of which cyclic groups, defined by the linkage of R^{a} and R^{b}, are optionally substituted by one or more substituents selected from ═O, ═NOR^{7b} and E²;
R³ and R⁴ independently represent hydrogen or a substituent selected from halo, —CN, R^{j1}, —OR^{j2}, —SR^{j3}, —N(R^{j4})R^{j5} and —C(O)OR^{j6};
R^{j1}, R^{j2}, R^{j3}, R^{j4}, R^{j5} and R^{j6} independently represent hydrogen or C₁₋₆ alkyl optionally substituted by one or more substituents selected from halo and —OR^{h};
R^{h} represents hydrogen or C₁₋₄ alkyl optionally substituted by one or more halo atoms;
R^{7a} and R^{7b} independently represent hydrogen or C₁₋₆ alkyl optionally substituted by one or more fluoro atoms;
each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)N(R^{10a})—OR^{11c}, —OC(═Y)—R^{10a}, —OC(═Y)—OR^{10a}, —OC(═Y)N(R^{10a})R^{11a}, —OS(O)₂O R^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
each R^{11c} independently represents, on each occasion when used herein, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶);
each R^{10a}, R^{11a} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶); or
any relevant pair of R^{10a}, R^{11a} and R^{12a} may be linked together to form a 4- to 20-membered ring, optionally containing one or more heteroatoms, optionally containing one or more unsaturations, and which ring is optionally substituted by one or more substituents selected from ═O, ═S, ═N(R²⁰) and E⁷;
each E¹, E², E³, E⁴, E⁵, E⁶ and E⁷ independently represents, on each occasion when used herein:
(i) Q⁴;
(ii) C₁₋₁₂ alkyl optionally substituted by one or more substituents selected from ═O and Q⁵; or
any two E¹, E², E³, E⁴, E⁵, E⁶ or E⁷ groups may be linked together to form a 3- to 12-membered ring, optionally containing one or more unsaturations, and which ring is optionally substituted by one or more substituents selected from ═O and J¹;
each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —C(═Y)N(R²⁰)—O—R^{21a}, —C(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;
each R^{21a} independently represents, on each occasion when used herein, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may be linked together to a 4- to 20-membered ring, optionally containing one or more heteroatoms, optionally containing one or more unsaturations, and which ring is optionally substituted by one or more substituents selected from J⁶ and ═O;
each J¹, J², J³, J⁴, J⁵ and J⁶ independently represents, on each occasion when used herein:
(i) Q⁷;
(ii) C₁₋₆ alkyl or heterocycloalkyl, both of which are optionally substituted by one or more substituents selected from ═O and Q⁸;
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰)R⁵¹, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may be linked together to form, a 3- to 8-membered ring, optionally containing one or more heteroatoms, optionally containing one or more unsaturations, and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically acceptable ester, amide, solvate or salt thereof,
provided that when R² represents —NH₂, R³ represents —CN and R⁴ represents hydrogen, then R¹ does not represent unsubstituted phenyl.

2. A compound as claimed in claim 1, wherein R² represents one of the following fragments: 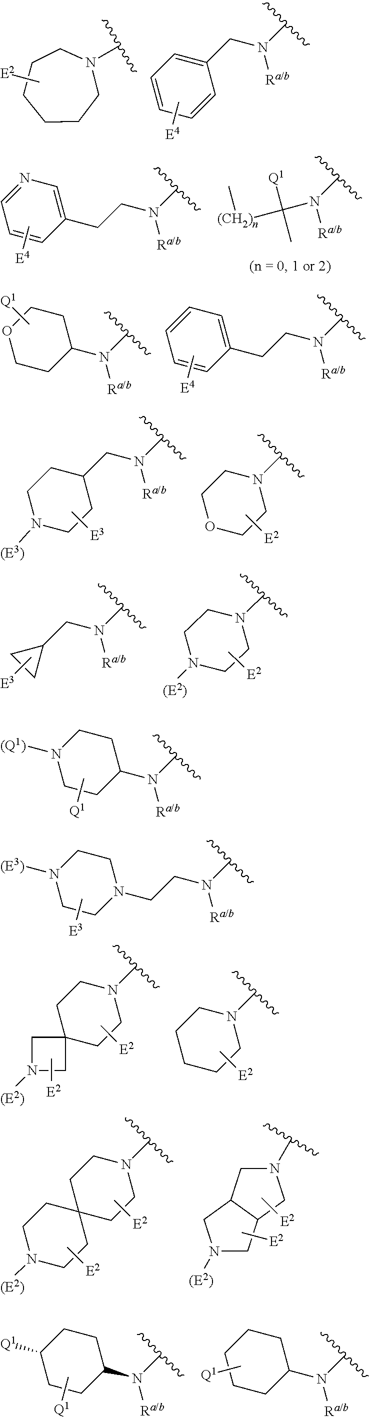 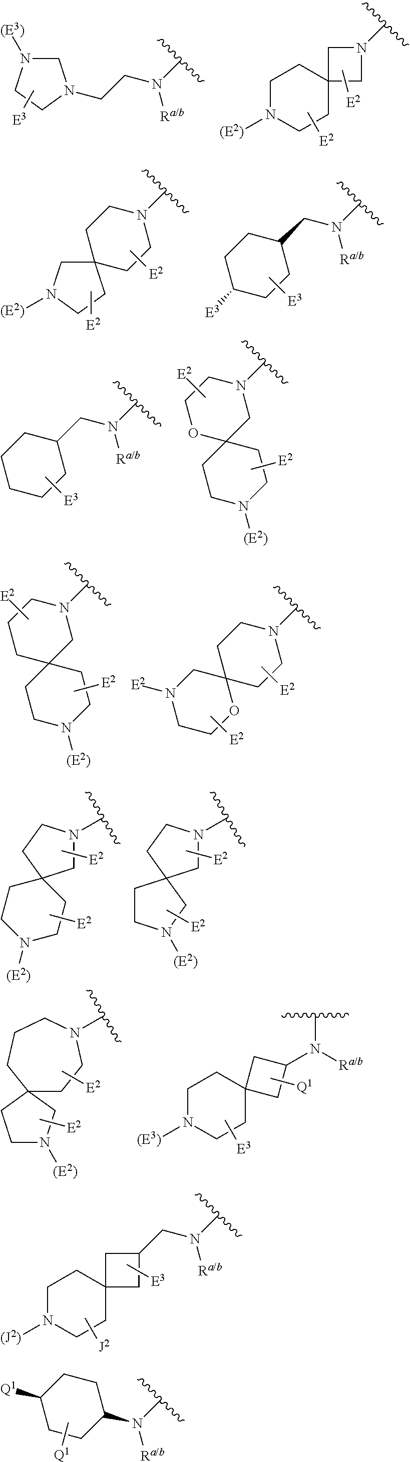
wherein the squiggly line represents the point of attachment to the requisite triazolopyridine bicycle of the compound of formula I, R^{a/b} represents R^{a} or R^{b}, and the other integers are as defined in claim 1.

3. A compound as claimed in claim 1, wherein: R¹ represents a 5- or 6-membered heteroaryl group (optionally substituted as defined herein) or, especially, aryl optionally substituted by one or more substituent(s) selected from E¹, in which the E¹ substituents are as herein defined (or, two E¹ substituents on the aryl ring may be linked together as defined herein); R^{j1}, R^{j2}, R^{j3}, R^{j4}, R^{j4}, R^{j5} and R^{j6} independently represent hydrogen or C₁₋₄ alkyl; Q¹ and Q² independently represent(s) —OR^{10a}; —N(R^{10a})R^{11a}; —C(═Y)N(R^{10a})R^{11a}; —N(R^{12a})C(═)R^{11a}; —C(═Y)OR^{10a}; —S(O)₂R^{10a}; aryl or heteroaryl, both of which are optionally substituted by one or more substituents selected from E⁴; heterocycloalkyl optionally substituted by one or more substituent(s) selected from ═O and E³; or C₃₋₆ cycloalkyl, which group may be unsubstituted or is optionally substituted by one or more E³ substituents; R^{10a}, R^{11a} and R^{12a} independently represent H or C₁₋₆ alkyl optionally substituted by one or more groups selected from ═O and E⁵; E¹ to E⁷ independently represent Q⁴ or C₁₋₆ alkyl optionally substituted by one or more Q⁵ substituents; any two E¹ to E⁷ substituents are not linked together; Q⁴ and Q⁵ independently represent —S(O)₂R²⁰, —N(R²²)—S(O)₂R²⁰ or halo, —CN, —OR²⁰, —N(R²⁰)R²¹, —C(═Y)R²⁰, —C(═Y)OR²⁰, —N(R²²)C(═Y)R²¹, —(═Y)N(R²⁰)R²¹, —S(O)₂N(R²⁰)R²¹₂NH₂), C₁₋₆ alkyl, heterocycloalkyl or heteroaryl; Y and Y^{a} independently represent ═S or ═O; R²⁰ and R²¹ independently represent hydrogen, C₁₋₄ alkyl, which latter group is optionally substituted by one or more substituent(s) selected from J⁴; when there is a —N(R²⁰)R²¹ moiety present, then one of R²⁰ and R²¹ represents hydrogen, and the other represents hydrogen or C₁₋₄ alkyl, which latter group is optionally substituted by one or more substituent(s) selected from J⁴; R²² represents hydrogen and C₁₋₃ alkyl; J¹ to J⁶ and independently represent Q⁷ or C₁₋₆ alkyl; Q⁷ and Q⁸ independently represent halo, C(═Y^{a})N(R⁵⁰)R⁵¹ or —C(═Y^{a})—R⁵⁰; and/or R⁵⁰ and R⁵¹ independently represent C₁₋₆ alkyl.

4. A compound as claimed in claim 1, wherein: one of R^{a} and R^{b} represents hydrogen and the other represents a substituent other than hydrogen; and when R^{a} or R^{b} represents a substituent other than hydrogen, then it is: (i) acyclic C₁₋₄ alkyl optionally substituted by one or more substituent(s) selected from Q¹; (ii) C₃₋₇ cycloalkyl optionally substituted by one or more substituent(s) selected from Q¹; or (iii) heterocycloalkyl, which heterocycloalkyl group is optionally substituted by one or more substituent(s) selected from Q¹; or R^{a} and R^{b} are linked together to form: a (first) 4- to 7-membered ring, which ring is optionally substituted by one or more substituent(s) selected from ═O and E², and, further, such a first ring may optionally be either: (i) fused to another 5- to 7-membered heterocycloalkyl group; or, (ii) the first ring may be linked via a single atom to another 4- to 6-membered carbocyclic or heterocycloalkyl group, to form a spiro cycle, which second ring of the spiro cycle may be substituted by one or more substituents selected from E² and, if applicable, ═O; and/or R³ and R⁴ independently represent hydrogen, —CN, halo and —C(O)OR^{j6}.

5. (canceled)

6. A pharmaceutical formulation including a compound of formula I 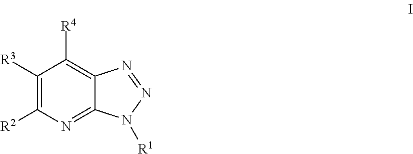
wherein:
R¹ represents aryl or heteroaryl, both of which are optionally substituted by one or more substituents selected from E¹;
R² represents a fragment of formula IA, 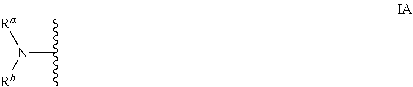
wherein R^{a} and R^{b} independently represent H, —C(O)—C₁₋₁₁ alkyl, —S(O)₂—C₁₋₁₁ alkyl, C₁₋₁₂ alkyl, heterocycloalkyl (which latter four groups are optionally substituted by one or more substituents selected from ═O, ═NOR^{7a} and Q¹), aryl or heteroaryl (which latter two groups are optionally substituted by one or more substituents selected from Q²); or
R^{a} and R^{b} are linked together, along with the requisite nitrogen atom to which they are necessarily attached, to form a (first) 3- to 7-membered cyclic group, optionally containing one further heteroatom selected from nitrogen, sulfur and oxygen, and which ring optionally:
(a) is fused to a second ring that is either a 3- to 7-membered saturated heterocycloalkyl group containing one to four heteroatoms selected from oxygen, sulfur and nitrogen, a 3- to 12-membered saturated carbocyclic ring, or an unsaturated 5- to 12-membered carbocyclic or heterocyclic ring;
(b) comprises a linker group —(C(R^{x})₂)ₚ— and/or —(C(R^{x})₂)ᵣ—O—(C(R^{x})₂)ₛ— (wherein p is 1 or 2; r is 0 or 1; s is 0 or 1; and each R^{x} independently represents hydrogen or C₁₋₆ alkyl), linking together any two non-adjacent atoms of the first 3- to 7-membered ring; or
(c) comprises a second ring that is either a 3- to 12-membered saturated carbocyclic ring or a 3- to 7-membered saturated heterocycloalkyl group containing one to four heteroatoms selected from oxygen and nitrogen, and which second ring is linked together with the first ring via a single carbon atom common to both rings,
all of which cyclic groups, defined by the linkage of R^{a} and R^{b}, are optionally substituted by one or more substituents selected from ═O, ═NOR^{7b} and E²;
R³ and R⁴ independently represent hydrogen or a substituent selected from halo, —CN, —R^{j1}, —OR^{j2}, —SR^{j3}, —N(R^{j4})R^{j5} and —C(O)OR^{j6};
R^{j1}, R^{j2}, R^{j3}, R^{j4}, R^{j5} and R^{j6} independently represent hydrogen or C₁₋₆ alkyl optionally substituted by one or more substituents selected from halo and —OR^{h};
R^{h} represents hydrogen or C₁₋₄ alkyl optionally substituted by one or more halo atoms;
R^{7a} and R^{7b} independently represent hydrogen or C₁₋₆ alkyl optionally substituted by one or more fluoro atoms;
each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)N(R^{10a})—OR^{11c}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
each R^{11c} independently represents, on each occasion when used herein, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶);
each R^{10a}, R^{11a} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶); or
any relevant pair of R^{10a}, R^{11a} and R^{12a} may be linked together to form a 4- to 20-membered ring, optionally containing one or more heteroatoms, optionally containing one or more unsaturations, and which ring is optionally substituted by one or more substituents selected from ═O, ═S, ═N(R²⁰) and E⁷;
each E¹, E², E³, E⁴, E⁵, E⁶ and E⁷ independently represents, on each occasion when used herein:
i) Q⁴;
(ii) C₁₋₁₂ alkyl optionally substituted by one or more substituents selected from ═O and Q⁵; or
any two E¹, E², E³, E⁴, E⁵, E⁶ or E⁷ groups may be linked together to form a 3- to 12-membered ring, optionally containing one or more unsaturations, and which ring is optionally substituted by one or more substituents selected from ═O and J¹;
each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —C(═Y)N(R²⁰)—O—R^{21a}, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;
each R^{21a} independently represents, on each occasion when used herein, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may be linked together to a 4- to 20-membered ring, optionally containing one or more heteroatoms, optionally containing one or more unsaturations, and which ring is optionally substituted by one or more substituents selected from J⁶ and ═O;
each J¹, J², J³, J⁴, J⁵ and J⁶ independently represents, on each occasion when used herein:
(i) Q⁷;
(ii) C₁₋₆ alkyl or heterocycloalkyl, both of which are optionally substituted by one or more substituents selected from ═O and Q⁸;
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰)R²¹, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may be linked together to form, a 3- to 8-membered ring, optionally containing one or more heteroatoms, optionally containing one or more unsaturations, and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically acceptable ester, amide, solvate or salt thereof, in admixture with a pharmaceutically acceptable adjuvant, diluent or carrier.

7-9. (canceled)

10. A method of treatment of a disease in which inhibition of PIM-1, PIM-2, PIM-3 and/or Flt3 is desired and/or required, which method comprises administration of a therapeutically effective amount of a compound of formula I 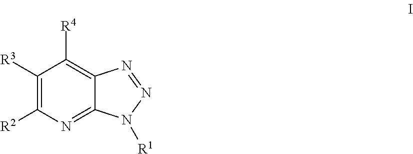
wherein:
R¹ represents aryl or heteroaryl, both of which are optionally substituted by one or more substituents selected from E¹;
R² represents a fragment of formula IA, 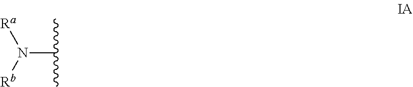
wherein R^{a} and R^{b} independently represent H, —C(O)—C₁₋₁₁ alkyl, —S(O)₂—C₁₋₁₁ alkyl, C₁₋₁₂ alkyl, heterocycloalkyl (which latter four groups are optionally substituted by one or more substituents selected from ═O, ═NOR^{7a} and Q¹), aryl or heteroaryl (which latter two groups are optionally substituted by one or more substituents selected from Q²); or
R^{a} and R^{b} are linked together, along with the requisite nitrogen atom to which they are necessarily attached, to form a (first) 3- to 7-membered cyclic group, optionally containing one further heteroatom selected from nitrogen, sulfur and oxygen, and which ring optionally:
(a) is fused to a second ring that is either a 3- to 7-membered saturated heterocycloalkyl group containing one to four heteroatoms selected from oxygen, sulfur and nitrogen, a 3- to 12-membered saturated carbocyclic ring, or an unsaturated 5- to 12-membered carbocyclic or heterocyclic ring;
(b) comprises a linker group —(C(R^{x})₂)ₚ— and/or —(C(R^{x})₂)ᵣ—O—(C(R^{x})₂)ₛ— (wherein p is 1 or 2; r is 0 or 1; s is 0 or 1; and each R^{x} independently represents hydrogen or C₁₋₆ alkyl), linking together any two non-adjacent atoms of the first 3- to 7-membered ring; or
(c) comprises a second ring that is either a 3- to 12-membered saturated carbocyclic ring or a 3- to 7-membered saturated heterocycloalkyl group containing one to four heteroatoms selected from oxygen and nitrogen, and which second ring is linked together with the first ring via a single carbon atom common to both rings,
all of which cyclic groups, defined by the linkage of R^{a} and R^{b}, are optionally substituted by one or more substituents selected from ═O, ═NOR^{7b} and E²;
R³ and R⁴ independently represent hydrogen or a substituent selected from halo, —CN, R^{j1}, —OR^{j2}, —SR^{j3}, —N(R^{j4})R^{j5} and —C(O)OR^{j6};
R^{j1}, R^{j2}, R^{j3}, R^{j4}, R^{j5} and R^{j6} independently represent hydrogen or C₁₋₆ alkyl optionally substituted by one or more substituents selected from halo and —OR^{h};
R^{h} represents hydrogen or C₁₋₄ alkyl optionally substituted by one or more halo atoms;
R^{7a} and R^{7b} independently represent hydrogen or C₁₋₆ alkyl optionally substituted by one or more fluoro atoms;
each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)N(R^{10a})—OR^{11c}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
each R^{11c} independently represents, on each occasion when used herein, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶);
each R^{10a}, R^{11a} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶); or
any relevant pair of R^{10a}, R^{11a} and R^{12a} may be linked together to form a 4- to 20-membered ring, optionally containing one or more heteroatoms, optionally containing one or more unsaturations, and which ring is optionally substituted by one or more substituents selected from ═O, ═S, ═N(R²⁰) and E⁷;
each E¹, E², E³, E⁴, E⁵, E⁶ and E⁷ independently represents, on each occasion when used herein:
(i) Q⁴;
(ii) C₁₋₁₂ alkyl optionally substituted by one or more substituents selected from ═O and Q⁵; or
any two E¹, E², E³, E⁴, E⁵, E⁶ or E⁷ groups may be linked together to form a 3- to 12-membered ring, optionally containing one or more unsaturations, and which ring is optionally substituted by one or more substituents selected from ═O and J¹;
each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —C(═Y)N(R²⁰)—O—R^{21a}, —OC(═Y)—R²⁰, —C(═Y)—OR²⁰, —OC(═Y)N(R²⁰)R²¹, —OS(O)₂OR²⁰, —OP(═Y)(OR²⁰)(OR²¹), —OP(OR²)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;
each R^{21a} independently represents, on each occasion when used herein, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may be linked together to a 4- to 20-membered ring, optionally containing one or more heteroatoms, optionally containing one or more unsaturations, and which ring is optionally substituted by one or more substituents selected from J⁶ and ═O;
each J¹, J², J³, J⁴, J⁵ and J⁶ independently represents, on each occasion when used herein:
(i) Q⁷;
(ii) C₁₋₆ alkyl or heterocycloalkyl, both of which are optionally substituted by one or more substituents selected from ═O and Q⁸;
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰)R⁵¹, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may be linked together to form, a 3- to 8-membered ring, optionally containing one or more heteroatoms, optionally containing one or more unsaturations, and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically-acceptable ester, amide, solvate or salt thereof, to a patient suffering from, or susceptible to, such a condition.

11. A combination product comprising:
(A) a compound of formula I 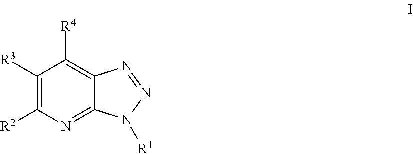
wherein:
R¹ represents aryl or heteroaryl, both of which are optionally substituted by one or more substituents selected from E¹;
R² represents a fragment of formula IA, 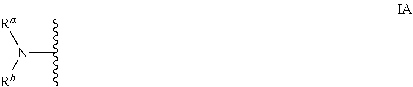
wherein R^{a} and R^{b} independently represent H, —C(O)—C₁₋₁₁ alkyl, —S(O)₂—C₁₋₁₁ alkyl, C₁₋₁₂ alkyl, heterocycloalkyl (which latter four groups are optionally substituted by one or more substituents selected from ═O, ═NOR^{7a} and Q¹), aryl or heteroaryl (which latter two groups are optionally substituted by one or more substituents selected from Q²); or
R^{a} and R^{b} are linked together, along with the requisite nitrogen atom to which they are necessarily attached, to form a (first) 3- to 7-membered cyclic group, optionally containing one further heteroatom selected from nitrogen, sulfur and oxygen, and which ring optionally:
(a) is fused to a second ring that is either a 3- to 7-membered saturated heterocycloalkyl group containing one to four heteroatoms selected from oxygen, sulfur and nitrogen, a 3- to 12-membered saturated carbocyclic ring, or an unsaturated 5- to 12-membered carbocyclic or heterocyclic ring;
(b) comprises a linker group —(C(R^{x})₂)ₚ— and/or —C(R^{x})₂)ᵣ—O—(C(R^{x})₂)ₛ— (wherein p is 1 or 2; r is 0 or 1; s is 0 or 1; and each R^{x} independently represents hydrogen or C₁₋₆ alkyl), linking together any two non-adjacent atoms of the first 3- to 7-membered ring; or
(c) comprises a second ring that is either a 3- to 12-membered saturated carbocyclic ring or a 3- to 7-membered saturated heterocycloalkyl group containing one to four heteroatoms selected from oxygen and nitrogen, and which second ring is linked together with the first ring via a single carbon atom common to both rings,
all of which cyclic groups, defined by the linkage of R^{a} and R^{b}, are optionally substituted by one or more substituents selected from ═O, ═NOR^{7b} and E²;
R³ and R⁴ independently represent hydrogen or a substituent selected from halo, —CN, R^{j1}, —OR^{j2}, —SR^{j3}, —N(R^{j4})R^{j5} and —C(O)OR^{j6};
R^{j1}, R^{j2}, R^{j3}, R^{j4}, R^{j5} and R^{j6} independently represent hydrogen or C₁₋₆ alkyl optionally substituted by one or more substituents selected from halo and —OR^{h};
R^{h} represents hydrogen or C₁₋₄ alkyl optionally substituted by one or more halo atoms;
R^{7a} and R^{7b} independently represent hydrogen or C₁₋₆ alkyl optionally substituted by one or more fluoro atoms;
each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)N(R^{10a})—OR^{11c}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR^{12a}S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
each R^{11c} independently represents, on each occasion when used herein, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶);
each R^{10a}, R^{11a} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶); or
any relevant pair of R^{10a}, R^{11a} and R^{12a} may be linked together to form a 4- to 20-membered ring, optionally containing one or more heteroatoms, optionally containing one or more unsaturations, and which ring is optionally substituted by one or more substituents selected from ═O, ═S, ═N(R²⁰) and E⁷;
each E¹, E², E³, E⁴, E⁵, E⁶ and E⁷ independently represents, on each occasion when used herein:
(i) Q⁴;
(ii) C₁₋₁₂ alkyl optionally substituted by one or more substituents selected from ═O and Q⁵; or
any two E¹, E², E³, E⁴, E⁵, E⁶ or E⁷ groups may be linked together to form a 3- to 12-membered ring, optionally containing one or more unsaturations, and which ring is optionally substituted by one or more substituents selected from ═O and J¹;
each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —C(═Y)N(R²)—O—R^{21a}, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;
each R^{21a} independently represents, on each occasion when used herein, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may be linked together to a 4- to 20-membered ring, optionally containing one or more heteroatoms, optionally containing one or more unsaturations, and which ring is optionally substituted by one or more substituents selected from J⁶ and ═O;
each J¹, J², J³, J⁴, J⁵ and J⁶ independently represents, on each occasion when used herein:
(i) Q⁷;
(ii) C₁₋₆ alkyl or heterocycloalkyl, both of which are optionally substituted by one or more substituents selected from ═O and Q⁸;
each Q⁷ and Q⁸ independently represents, on each occasion when used herein:
halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰)R⁵¹, —S(O)₂R²⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁰ and —N(R⁶¹)R⁶²; or
any relevant pair of R⁵⁰, R⁵¹ and R⁵² may be linked together to form, a 3- to 8-membered ring, optionally containing one or more heteroatoms, optionally containing one or more unsaturations, and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically-acceptable ester, amide, solvate or salt thereof; and
(B) another therapeutic agent that is useful in the treatment of in the treatment of cancer and/or a proliferative disease,
wherein each of components (A) and (B) is formulated in admixture with a pharmaceutically-acceptable adjuvant, diluent or carrier.

12. A process for the preparation of a compound of formula I as defined in claim 1, which process comprises:
(i) reaction of a compound of formula II, 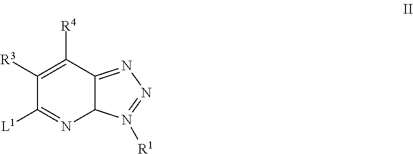
wherein L¹ represents a suitable leaving group, and R¹, R³ and R⁴ are as defined in claim 1, with a compound of formula III,
R²—H III
wherein R² is as hereinbefore defined in claim 1;
(ii) for compounds of formula I in which R³ represents halo, reaction of a compound corresponding to a compound of formula I in which R³ represents hydrogen, with a reagent that is a source of halide ions;
(iii) for compounds of formula I in which R³ and/or R⁴ represents a substituent other that hydrogen or halo, reaction of a corresponding compound of formula I, in which R³ and/or R⁴ represents halo, with a compound of formula IV,
R^{3a}-L² IV
wherein R^{3a} represents R³ and/or R⁴ as defined in claim 1 provided that it does not represent hydrogen or halo, and L² represents hydrogen or a suitable leaving group;
(iv) reaction of a compound of formula V, 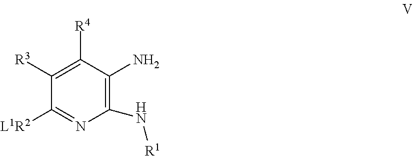
wherein L¹R² represents either L¹ or R³, and R¹, R³ and R⁴ are as defined in claim 1, with sodium nitrite in the presence of acetic acid and water, followed by, if necessary, reaction with a compound of formula III as defined above.

13. A process for the preparation of a pharmaceutical formulation as defined in claim 6, which process comprises bringing into association a compound of formula I, or a pharmaceutically acceptable ester, amide, solvate or salt thereof, but without the proviso, with a pharmaceutically-acceptable adjuvant, diluent or carrier.

14. A process for the preparation of a combination product as defined in claim 11, which process comprises bringing into association a compound of formula I 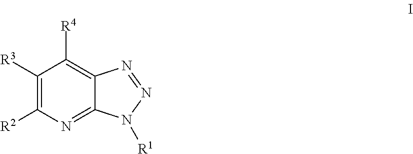
wherein:
R¹ represents aryl or heteroaryl, both of which are optionally substituted by one or more substituents selected from E¹;
R² represents a fragment of formula IA, 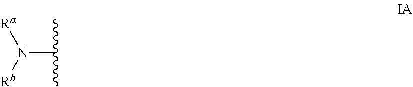
wherein R^{a} and R^{b} independently represent H, —C(O)—C₁₋₁₁ alkyl, —S(O)₂—C₁₋₁₁ alkyl, C₁₋₁₂ alkyl, heterocycloalkyl (which latter four groups are optionally substituted by one or more substituents selected from ═O, ═NOR^{7a} and Q¹), aryl or heteroaryl (which latter two groups are optionally substituted by one or more substituents selected from Q²); or
R^{a} and R^{b} are linked together, along with the requisite nitrogen atom to which they are necessarily attached, to form a (first) 3- to 7-membered cyclic group, optionally containing one further heteroatom selected from nitrogen, sulfur and oxygen, and which ring optionally:
(a) is fused to a second ring that is either a 3- to 7-membered saturated heterocycloalkyl group containing one to four heteroatoms selected from oxygen, sulfur and nitrogen, a 3- to 12-membered saturated carbocyclic ring, or an unsaturated 5- to 12-membered carbocyclic or heterocyclic ring;
(b) comprises a linker group —(C(R^{x})₂)ₚ— and/or —(C(R^{x})₂)ᵣ—O—(C(R^{x})₂)ₛ— (wherein p is 1 or 2; r is 0 or 1; s is 0 or 1; and each R^{x} independently represents hydrogen or C₁₋₆ alkyl), linking together any two non-adjacent atoms of the first 3- to 7-membered ring; or
(c) comprises a second ring that is either a 3- to 12-membered saturated carbocyclic ring or a 3- to 7-membered saturated heterocycloalkyl group containing one to four heteroatoms selected from oxygen and nitrogen, and which second ring is linked together with the first ring via a single carbon atom common to both rings,
all of which cyclic groups, defined by the linkage of R^{a} and R^{b}, are optionally substituted by one or more substituents selected from ═O, ═NOR^{7b} and E²;
R³ and R⁴ independently represent hydrogen or a substituent selected from halo, —CN, R^{j1}, —OR^{j2}, —SR^{j3}, —N(R^{j4})R^{j5} and —C(O)OR^{j6};
R^{j1}, R^{j2}, R^{j3}, R^{j4}, R^{j5} and R^{j6} independently represent hydrogen or C₁₋₆ alkyl optionally substituted by one or more substituents selected from halo and —OR^{h};
R^{h} represents hydrogen or C₁₋₄ alkyl optionally substituted by one or more halo atoms;
R^{7a} and R^{7b} independently represent hydrogen or C₁₋₆ alkyl optionally substituted by one or more fluoro atoms;
each Q¹ and Q² independently represents, on each occasion when used herein: halo, —CN, —NO₂, —N(R^{10a})R^{11a}, —OR^{10a}, —C(═Y)—R^{10a}, —C(═Y)—OR^{10a}, —C(═Y)N(R^{10a})R^{11a}, —C(═Y)N(R^{10a})—OR^{11c}, —OC(═Y)—R^{10a}, —OC(═Y)—OR^{10a}, —OC(═Y)N(R^{10a})R^{11a}, —OS(O)₂OR^{10a}, —OP(═Y)(OR^{10a})(OR^{11a}), —OP(OR^{10a})(OR^{11a}), —N(R^{12a})C(═Y)R^{11a}, —N(R^{12a})C(═Y)OR^{11a}, —N(R^{12a})C(═Y)N(R^{10a})R^{11a}, —NR^{12a}S(O)₂R^{10a}, —NR¹²S(O)₂N(R^{10a})R^{11a}, —S(O)₂N(R^{10a})R^{11a}, —SC(═Y)R^{10a}, —S(O)₂R^{10a}, —SR^{10a}, —S(O)R^{10a}, C₁₋₁₂ alkyl, heterocycloalkyl (which latter two groups are optionally substituted by one or more substituents selected from ═O, ═S, ═N(R^{10a}) and E³), aryl or heteroaryl (which latter two groups are optionally substituted by one or more substituents selected from E⁴);
each R^{11c} independently represents, on each occasion when used herein, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶);
each R^{10a}, R^{11a} and R^{12a} independently represent, on each occasion when used herein, hydrogen, C₁₋₁₂ alkyl, heterocycloalkyl (which latter two groups are optionally substituted by one or more substituents selected from ═O, ═S, ═N(R²⁰) and E⁵), aryl or heteroaryl (which latter two groups are optionally substituted by one or more substituents selected from E⁶); or
any relevant pair of R^{10a}, R^{11a} and R^{12a} may be linked together to form a 4- to 20-membered ring, optionally containing one or more heteroatoms, optionally containing one or more unsaturations, and which ring is optionally substituted by one or more substituents selected from ═O, ═S, ═N(R²⁰) and E⁷;
each E¹, E², E³, E⁴, E⁵, E⁶ and E⁷ independently represents, on each occasion when used herein:
(i) Q⁴;
(ii) C₁₋₁₂ alkyl optionally substituted by one or more substituents selected from ═O and Q⁵; or
any two E¹, E², E³, E⁴, E⁵, E⁶ or E⁷ groups may be linked together to form a 3- to 12-membered ring, optionally containing one or more unsaturations, and which ring is optionally substituted by one or more substituents selected from ═O and J¹;
each Q⁴ and Q⁵ independently represent, on each occasion when used herein: halo, —CN, —NO₂, —N(R²⁰)R²¹, —OR²⁰, —C(═Y)—R²⁰, —C(═Y)—OR²⁰, —C(═Y)N(R²⁰)R²¹, —C(═Y)N(R²⁰)—O—R^{21a}, —OC(═Y)—R²⁰, —OC(═Y)—OR²⁰, —OC(═Y)N(R²⁰)R²¹, —OS(O)₂OR²⁰, —OP(═Y)(OR²⁰)(OR²¹), —OP(OR²⁰)(OR²¹), —N(R²²)C(═Y)R²¹, —N(R²²)C(═Y)OR²¹, —N(R²²)C(═Y)N(R²⁰)R²¹, —NR²²S(O)₂R²⁰, —NR²²S(O)₂N(R²⁰)R²¹, —S(O)₂N(R²⁰)R²¹, —SC(═Y)R²⁰, —S(O)₂R²⁰, —SR²⁰, —S(O)R²⁰, C₁₋₆ alkyl, heterocycloalkyl (which latter two groups are optionally substituted by one or more substituents selected from ═O and J²), aryl or heteroaryl (which latter two groups are optionally substituted by one or more substituents selected from J³);
each Y independently represents, on each occasion when used herein, ═O, ═S, ═NR²³ or ═N—CN;
each R^{21a} independently represents, on each occasion when used herein, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵);
each R²⁰, R²¹, R²² and R²³ independently represent, on each occasion when used herein, hydrogen, C₁₋₆ alkyl, heterocycloalkyl (which latter two groups are optionally substituted by one or more substituents selected from J⁴ and ═O), aryl or heteroaryl (which latter two groups are optionally substituted by one or more substituents selected from J⁵); or
any relevant pair of R²⁰, R²¹ and R²², may be linked together to a 4- to 20-membered ring, optionally containing one or more heteroatoms, optionally containing one or more unsaturations, and which ring is optionally substituted by one or more substituents selected from J⁶ and ═O;
each J¹, J², J³, J⁴, J⁵ and J⁶ independently represents, on each occasion when used herein:
(i) Q⁷;
(ii) C₁₋₆ alkyl or heterocycloalkyl, both of which are optionally substituted by one or more substituents selected from ═O and Q⁸;
each Q⁷ and Q⁸ independently represents, on each occasion when used herein: halo, —CN, —N(R⁵⁰)R⁵¹, —OR⁵⁰, —C(═Y^{a})—R⁵⁰, —C(═Y^{a})—OR⁵⁰, —C(═Y^{a})N(R⁵⁰)R⁵¹, —N(R⁵²)C(═Y^{a})R⁵¹, —NR⁵²S(O)₂R⁵⁰, —S(O)₂N(R⁵⁰)R⁵¹, —N(R⁵²)—C(═Y^{a})—N(R⁵⁰)R⁵¹, —S(O)₂R⁵⁰, —SR⁵⁰, —S(O)R⁵⁰ or C₁₋₆ alkyl optionally substituted by one or more fluoro atoms;
each Y^{a} independently represents, on each occasion when used herein, ═O, ═S, ═NR⁵³ or ═N—CN;
each R⁵⁰, R⁵¹, R⁵² and R⁵³ independently represents, on each occasion when used herein, hydrogen or C₁₋₆ alkyl optionally substituted by one or more substituents selected from fluoro, —OR⁶⁶ and —N(R⁶¹)R⁶²; or
any relevant pair of R⁵⁰, R⁵¹ and R⁵² may be linked together to form, a 3- to 8-membered ring, optionally containing one or more heteroatoms, optionally containing one or more unsaturations, and which ring is optionally substituted by one or more substituents selected from ═O and C₁₋₃ alkyl;
R⁶⁰, R⁶¹ and R⁶² independently represent hydrogen or C₁₋₆ alkyl optionally substituted by one or more fluoro atoms,
or a pharmaceutically acceptable ester, amide, solvate or salt thereof, with the other therapeutic agent that is useful in the treatment of cancer and/or a proliferative disease, and at least one pharmaceutically-acceptable adjuvant, diluent or carrier.

15. A method according to claim 10, wherein the disease is cancer, an immune disorder, a cardiovascular disease, a viral infection, inflammation, a metabolism/endocrine function disorder, a neurological disorder, an obstructive airways disease, an allergic disease, an inflammatory disease, immunosuppression, a disorder commonly connected with organ transplantation, an AIDS-related disease, benign prostate hyperplasia, familial adenomatosis, polyposis, neuro-fibromatosis, psoriasis, a bone disorder, atherosclerosis, vascular smooth cell proliferation associated with atherosclerosis, pulmonary fibrosis, arthritis glomerulonephritis and post-surgical stenosis, restenosis, stroke, diabetes, hepatomegaly, Alzheimer's disease, cystic fibrosis, a hormone-related disease, an immunodeficiency disorder, a destructive bone disorder, an infectious disease, a condition associated with cell death, thrombin-induced platelet aggregation, chronic myelogenous leukaemia, liver disease, a pathologic immune condition involving T cell activation, CNS disorders, and other associated diseases.
